(19) United States
(12) Patent Application Publication
(10) Pub. No.: US 2024/0197700 A1
(43) Pub. Date: Jun. 20, 2024

(54) Methods and Compositions for Treating Lysosomal Storage Disorders

(71) Applicant: Sanford Health, Sioux Falls, SD (US)

(72) Inventors: Jon Brudvig, Sioux Falls, SD (US); Jill Weimer, Sioux Falls, SD (US)

(21) Appl. No.: 18/284,825

(22) PCT Filed: Mar. 28, 2022
(86) PCT No.: PCT/US2022/022165

§ 371 Date: Sep. 28, 2023

Related U.S. Application Data
(60) Provisional application No. 63/167,385, filed on Mar. 29, 2021.

(51) Int. Cl. A61K 31/44; A61K 31/5375; A61K 38/26; A61K 45/06; A61P 3/00; A61P 25/28

(52) U.S. Cl. CPC A61K 31/44; A61K 31/5375; A61K 38/26; A61K 45/06; A61P 3/00; A61P 25/28

(57) ABSTRACT

The present disclosure provides methods and compositions for treating or limiting development of a lysosomal storage disorder, by administering to a subject that has or is at risk of a lysosomal storage disorder thereof an amount effective of a sortilin (SORT1) inhibitor to treat or limit development of the lysosomal storage disorder.

### CROSS REFERENCE

This application claims priority to U.S. Provisional Application Ser. No. 63/167,385 filed Mar. 29, 2021, incorporated herein by reference in its entirety.

### SEQUENCE LISTING STATEMENT

A computer readable form of the Sequence Listing is filed with this application by electronic submission and is incorporated into this application by reference in its entirety. The Sequence Listing is contained in the file created on Mar. 21, 2022 having the file name “21-0242-WO-SeqList_ST25.txt” and is 2 kb in size.

### BACKGROUND

Disorders believed to have a predominantly lysosomal etiology are often termed “lysosomal storage disorders (LSDs).” and include numerous rare genetic diseases that are most often inherited in a recessive manner. In all of these cases, restoration of lysosomal health and function has the potential to ameliorate a range of cellular and systemic pathologies.

The Neuronal Ceroid Lipofuscinoses (NCLs), also known as Batten Disease, are a group of LSDs characterized by the accumulation of autofluorescent storage material (ASM) in lysosomes, neuroinflammation and neurodegeneration, neurological symptoms and premature death. NCLs are caused by mutations in one of 13 genes and are typically inherited in an autosomal recessive manner. The causative Ceroid Lipofuscinosis Neuronal (CLN) genes encode proteins with diverse cellular functions, ranging from soluble lysosomal enzymes (e.g. CLN1 and CLN2) to molecular chaperones (e.g. CLN4) to transmembrane proteins with elusive function (e.g. CLN3) [1]. In all cases, lysosomal function is severely compromised. In disease caused by primary genetic defects in soluble lysosomal enzymes, initial accumulation of substrates specific to those enzymes is quickly followed by accumulation of diverse secondary substrates resulting from overall compromised lysosomal function. In NCLs, a hallmark of lysosomal dysfunction is the accumulation of ASM consisting largely of mitochondrial ATP synthase subunit C (SubC) and saposins A and D, along with a multitude of other substrates including lipids and lipidated proteins.

Dysfunctional lysosomes (and presumably other cellular components and pathways) are believed to initiate a cascade of neuroinflammation with glial activation and eventual neurodegeneration. While neurons are some of the first cells to be severely affected in NCLs, lysosomal pathology has been documented in a wide range of cell types including cardiac cells, skin fibroblasts, and leukocytes. Due to their severe lysosomal pathology, diverse cellular and molecular etiologies, and monogenic inheritance, NCLs are useful as models for LSDs and other diseases with a lysosomal component. For all of these disorders, strategies that can restore lysosomal function resulting from primary genetic defects or secondary dysfunction have the potential to serve as disease modifying therapies.

### SUMMARY

In one aspect, the disclosure provides method for treating or limiting development of a lysosomal storage disorder, comprising administering to a subject that has or is at risk of a lysosomal storage disorder thereof an amount effective of a sortilin (SORT1) inhibitor to treat or limit development of the lysosomal storage disorder. In one embodiment, the SORT1 inhibitor comprises a compound of the formula (I):

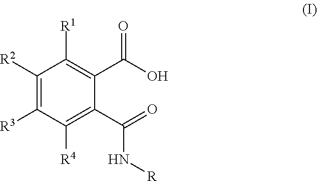

or pharmaceutically acceptable salts thereof, wherein
R¹ is hydrogen, halogen, C₁-C₆ alkyl, or C₁-C₆ haloalkyl;
R² is hydrogen, halogen, C₁-C₆ alkyl, C₁-C₆ haloalkyl, —NO₂, —CN, —OH, —SH, —NH₂, —NH(C₁-C₆ alkyl), —N(C₁-C₆ alkyl)₂, C₁-C₆ alkoxy, C₁-C₆ haloalkoxy, aryl optionally substituted with one or more R₅, or heteroaryl optionally substituted with one or more R₅;
R³ is hydrogen, halogen, C₁-C₆ alkyl, C₁-C₆ haloalkyl, —NO₂, —CN, —OH, —SH, —NH₂, —NH(C₁-C₆ alkyl), —N(C₁-C₆ alkyl)₂, C₁-C₆ alkoxy, or C₁-C₆ haloalkoxy;
R⁴ is hydrogen, halogen, C₁-C₆ alkyl, or C₁-C₆ haloalkyl; and
R is C₁-C₆ alkyl, aryl optionally substituted with one or more R₅, or heteroaryl optionally substituted with one or more R₅,
wherein
   each R₅ is independently selected from the group consisting of halogen, —NO₂, —CN, C₁-C₆ alkyl, C₁-C₆ haloalkyl, —OH, C₁-C₆ alkoxy, and C₁-C₆ haloalkoxy.

In another embodiment, the SORT1 inhibibitor comprises 2-((6-methylpyridin-2-yl)carbamoyl)-5-(trifluoromethyl)benzoic acid (AF38469), or a pharmaceutically acceptable salts thereof.

In various embodiments, the lysosomal storage disorder is selected from the group consisting of NCL/Batten Disease caused by mutations in CLN gene CLN1 (PPT1), CLN2 (TPP1), CLN3, CLN4 (DNAJC5), CLN5, CLN6, CLN7 (MFSD8), CLN8, CLN10 (CTSD). CLN11, CLN12 (ATP13A2), CLN13 (CTSF), CLN14 (KCTD7), CLCN6, and/or SGSH; Pompe disease, Fabry disease, Gaucher disease, Niemann-Pick disease Types A, B, and C; GM1 gangliosidosis, GM2 gangliosidosis (including Sandhoff and Tay-Sachs), mucopolysachariddoses (MPS) types I (Hurler disease)/II (Hunter disease)/IIIa (Sanfilippo A)/IIIB (Sanfilippo B)/IIIc (Sanfilippo C)/IIId (Sanfilippo D)/IVA (Morquio A)/IVB/VI/VII (Sly)/IX, mucolipisosis III (I-cell) and IV, multiple sulfatase deficiency; sialidosis, galactosialidosis, α-mannosidosis, β-mannosidosis, apartylglucosaminuria, fucosidosis, Schindler disease, metachromatic leukodystrophy caused by deficiencies in either arylsulfatase A or Saposin B, globoid cell leukodystrophy (Krabbe disease), Farber lipogranulomatosis, Wolman and cholesteryl ester storage disease. pycnodystostosis, cystinosis, Salla disease, Danon disease, Griscelli disease Types 1/2/3, Hermansky Pudliak Disease, and Chédiak-Higashi syndrome.

In another embodiment, the method further comprises administering to the subject an amount effective of a p75 neurotrophin receptor (NGFR) modulator, and/or a glucagon-like peptide-1 receptor (GLP-1R) agonist.

In one embodiment, the method comprises administering to the subject an amount effective of AF38469 (2-((6-methylpyridin-2-yl)carbamoyl)-5-(trifluoromethyl)benzoic acid) or a pharmaceutically acceptable salt thereof, and LM11A-31 (N-[2-(morpholin-4-yl)ethyl]-L-isoleucinamide) or a pharmaceutically acceptable salt thereof, to treat the and/or the lysosomal storage disorder. In another embodiment, the method further comprises administering to the subject an amount effective of semaglutide, or a pharmaceutically acceptable salt thereof, to treat the lysosomal storage disorder.

In another aspect, the disclosure provides pharmaceutical composition, comprising:
(a) a SORT1 inhibitor; and
(b) 1, 2, or all 3 of:
   (i) a NGFR modulator,
   (ii) a GLP-1R agonist; and
   (iii) a nucleic acid encoding a gene therapy expression product capable of substituting for a protein deficient in a lysosomal storage disorder and/or neurological disorder; and
(c) a pharmaceutically acceptable carrier.

In one embodiment the pharmaceutical composition comprises
(a) a SORT1 inhibitor;
(b) one or both of:
   (i) a NGFR modulator, and
   (ii) a GLP-1R agonist; and
(c) a pharmaceutically acceptable carrier.

In one embodiment, the composition comprises AF38469 (2-((6-methylpyridin-2-yl)carbamoyl)-5-(trifluoromethyl)benzoic acid) or a pharmaceutically acceptable salt thereof. In another embodiment, the composition further comprises LM11A-31 (N-[2-(morpholin-4-yl)ethyl]-L-isoleucinamide) or a pharmaceutically acceptable salt thereof. In a further embodiment, the composition further comprises semaglutide or a pharmaceutically acceptable salt thereof.

### DESCRIPTION OF THE FIGURES

FIG. 1(A-C). Treatment with AF38469 reduces pathology in Batten disease mouse embryonic fibroblasts. (A) NCL MEFs display elevated Lysotracker™ levels at DIV7. Treatment with 40 nM, 400 nM, and 4 μM AF38469 (2-((6-methylpyridin-2-yl)carbamoyl)-5-(trifluoromethyl)benzoic acid) significantly reduced LysoTracker™ signal in Cln1^{R151X}, Cln2^{R207X}, Cln6^{nclf}, Cln8^{mnd}, and Cln11^{-/-} MEFs. In Cln3^{Δex7/8} MEFs, AF38469 (40 nM, 400 nM, and 4 μM) significantly increased, significantly reduced, and had no impact on Lysotracker™ signal, respectively. n=650-4500 cells/treatment group. (B) NCL MEFs display elevated ASM levels at DIV7. Treatment with 40 nM, 400 nM, and 4 μM of AF38469 significantly reduced the accumulation of autofluorescent storage material (ASM) in Cln1^{R151X}, Cln2^{R207X}, Cln3^{Δex7/8}, Cln6^{nclf}, and Cln8^{mnd} MEFs. Treatment with AF38469 did not have an impact on Cln11^{-/-} MEFs. n=1000-4000 cells/treatment group. (C) Treatment with 40 nM, 400 nM and 4 μM of AF38469 did not significantly affect the viability of Cln1^{R151X}, Cln2^{R207X}, Cln6^{nclf}, Cln8^{mnd}, and Cln11^{-/-} MEFs. Treatment with 40 nM of AF8469 significantly increased viability in Cln3^{Δex7/8} MEFs while doses of 400 nM and 4 μM had no impact on viability. n=9 wells/treatment Scale bar=100 μm. For A-C, two-way ANOVA with a Scheffe post-hoc test *=significantly (p<0.05) different from wild type (WT) vehicle. #=significantly (p<0.05) different from NCL vehicle.

FIG. 1(A-C). Treatment with AF38469 reduces pathology in Batten disease primary neuronal cultures. (A) NCL primary neuronal cultures (PNCs) display elevated Lyostracker™ levels at DIV7. Treatment with 40 nM, 400 nM, and 4 μM AF38469 significantly reduced LysoTracker™ signal in Cln1^{R151X} and Cln6^{nclf} PNCs. In Cln2^{R207X} PNCs. AF38469 (4 μM) significantly reduced, while the 40 nM and 400 nM doses had no impact on LysoTracker™ signal. In Cln3^{Δex7/8} PNCs, AF38469 (40 nM, 400 nM, 4 μM) significantly increased, significantly reduced, and had no impact on LysoTracker™ signal, respectively. In Cln8^{mnd} PNCs, AF38469 (40 nM, 400 nM, 4 μM) significantly increased, significantly increased, and had no impact on Lysotracker™ signal, respectively. n=5000-20000 cells/treatment group. (B) NCL PNCs display elevated ASM levels at DIV7. Treatment with 40 nM, 400 nM, and 4 μM of AF38469 in significantly reduced the accumulation of autofluorescent storage material (ASM) in Cln1^{R151X}, Cln2^{R207X}, Cln3^{Δex7/8}, and Cln6^{nclf} PNCs. Treatment with AF38469 significantly increased ASM levels in Cln8^{mnd} PNCs. n=4000-20000 cells/treatment group. (C) Treatment with 40 nM, 400 nM and 4 μM of AF38469 did not significantly affect the viability of Cln1^{R151X}, Cln2^{R207X}, Cln6^{nclf}, and Cln8^{mnd} PNCs. Treatment with 40 nM of AF8469 significantly increased viability in Cln3^{Δex7/8} MEFs while doses of 400 nM and 4 μM had no impact on viability. n=9 wells/treatment Scale bar=100 μm, Inset scale bar=50 μm. For A-C, two-way ANOVA with a Scheffe post-hoc test *=significantly (p<0.05) different from wild type (WT) vehicle. #=significantly (p<0.05) different from NCL vehicle.

FIG. 2 (A-C). Treatment with AF38469 stimulates TFEB nuclear translocation as monitored via time course study. (A, B) Representative confocal images of transfected neuro 2A (N2A) cells, vehicle and AF38469 treated. Treatment with AF38469 stimulated transcription factor E3 (TFE3) nuclear translocation. These results were quantified in C. (C) Quantification of TFEB nuclear translocation after treatment with vehicle or AF38469 (40 nM, 400 nM). After 90 minutes of incubation with 40 nM of AF38469, there was a significant increase in TFEB nuclear translocation. After 180 minutes of incubation with 40 nM and 400 nM of AF38469, there was significant increase in TFEB nuclear translocation. n=5000-6500 cells/treatment Scale bar=50 μm, Inset scale bar=25 μm. One-way ANOVA. Mean±S.E.M. Dunnett's multiple comparisons test compared to the vehicle treated group. *p<0.05, **p<0.01, ***p<0.001, ****p<0.0001.

FIG. 3(A-C). Treatment with AF38469 stimulates TFE3 nuclear translocation as monitored via time course study. (A, B) Representative confocal images of transfected N2A cells, vehicle and AF38469 treated. Treatment with AF38469 stimulated transcription factor E3 (TFE3) nuclear translocation. These results were quantified in C. (C) Quantification of TFE3 nuclear translocation after treatment with vehicle or AF38469 (40 nM, 400 nM). After 90 minutes of incubation with 40 nM and 400 nM of AF38469, there was a significant increase in TFE3 nuclear translocation. After 180 minutes of incubation with 40 nM and 400 nM of AF38469, trends return to baseline for the 40 nM dose but a trend towards increased nuclear translocation remains for the 400 nM dose. n=6500-8500 cells/treatment. Scale bar=50 μm, Inset scale bar=25 μm. One-way ANOVA. Mean±S.E.M. Dunnett's multiple comparisons test compared to the vehicle treated group. *p<0.05, **p<0.01, ***p<0.001, ****p<0.0001.

FIG. 5(A-E). Comparative transcriptomic analysis of differentially expressed genes in AF38469 treated and vehicle treated wild type mouse embryonic fibroblasts. (A, B) AF38469 treated wild type MEFs show no distinct pattern of up or down regulation when comparing all lysosomal differentially expressed genes (A) or lysosomal genes that are not regulated by TFEB (B). (C) When TFEB target genes are examined in isolation. AF38469 treated WT MEFs show a distinct pattern of upregulation. (D) The significantly upregulated lysosomal genes in AF38469 treated WT MEFs that are regulated by TFEB organized by p-values. (E) The significantly upregulated lysosomal genes in AF38469 treated WT MEFs that are regulated by TFEB organized by fold change values. p-values are not corrected for multiple comparisons.

FIG. 4(A-B) Treatment with AF38469 increases PPT1 and TPP1 enzyme activity in Batten disease mouse embryonic fibroblasts. (A) Treatment with AF38469 (40 nM) significantly increased levels of palmitoyl-protein thioesterase 1 (PPT1) enzyme activity in wild type, Cln2^{R207X}, Cln3^{Δex7/8}, Cln6^{nclf}, and Cln11^{-/-} MEFs when compared to the vehicle treated mutant control. n=5 wells/treatment. (B) Treatment with AF38469 (40 nM) significantly increased levels of tripeptidyl peptidase 1 (TPP1) enzyme activity in wild type, Cln3^{Δex7/8}, Cln6^{nclf}, and Cln11^{-/-} MEFs when compared to the vehicle treated mutant control. Levels of TPP1 enzyme activity in Cln2^{R207X} were not altered by treatment of AF38469. n=5 wells/treatment. Two-way ANOVA. Mean±S.E.M. Tukey's multiple comparisons test. *p<0.05, **p<0.01, ***p<0.001, ****p<0.0001.

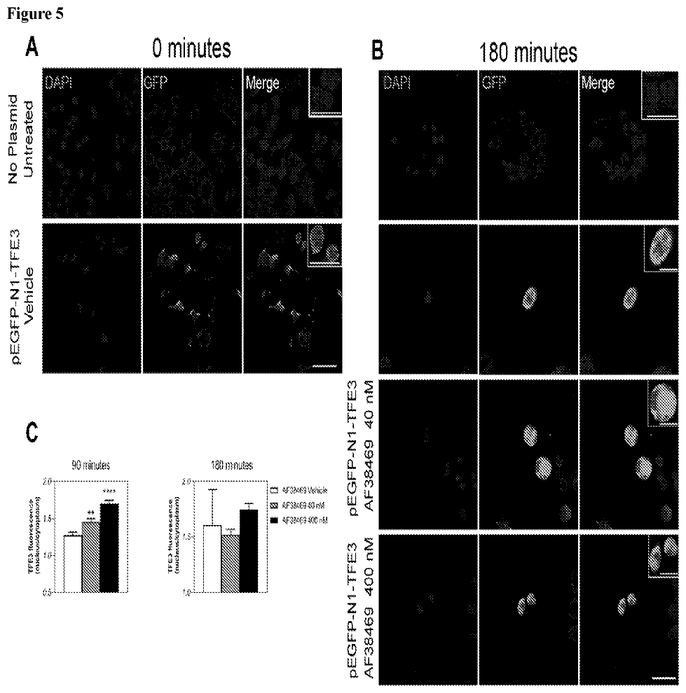

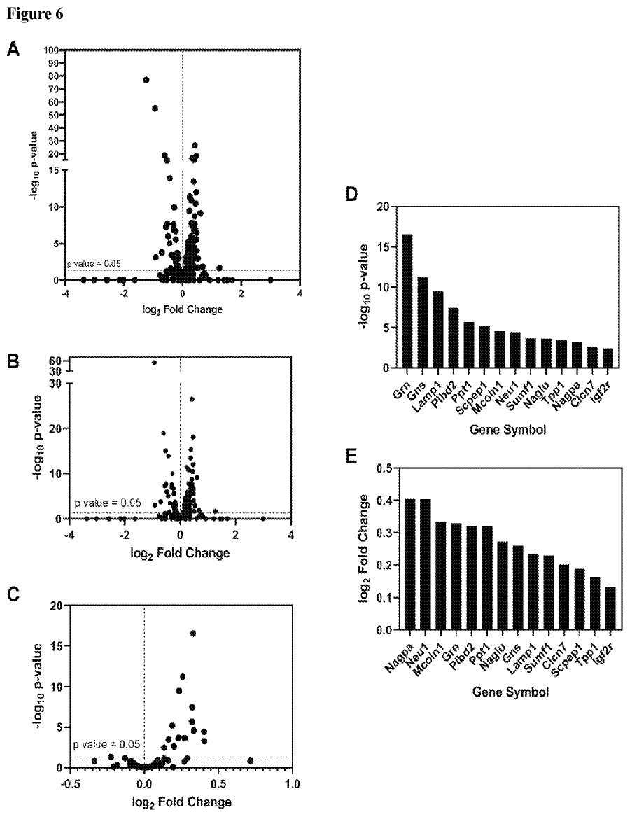

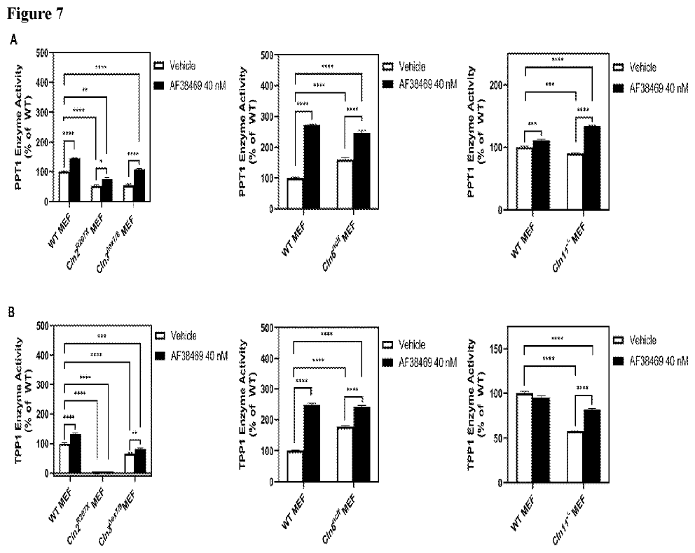

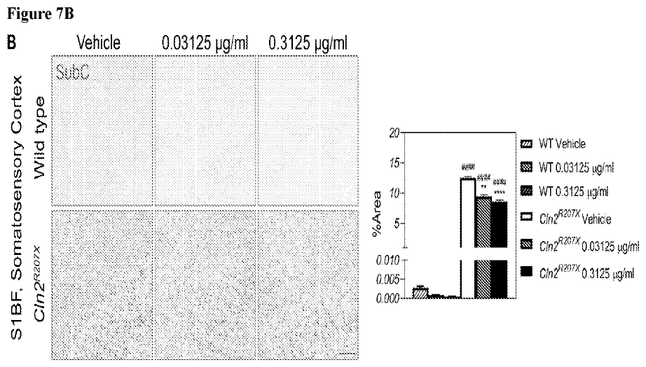

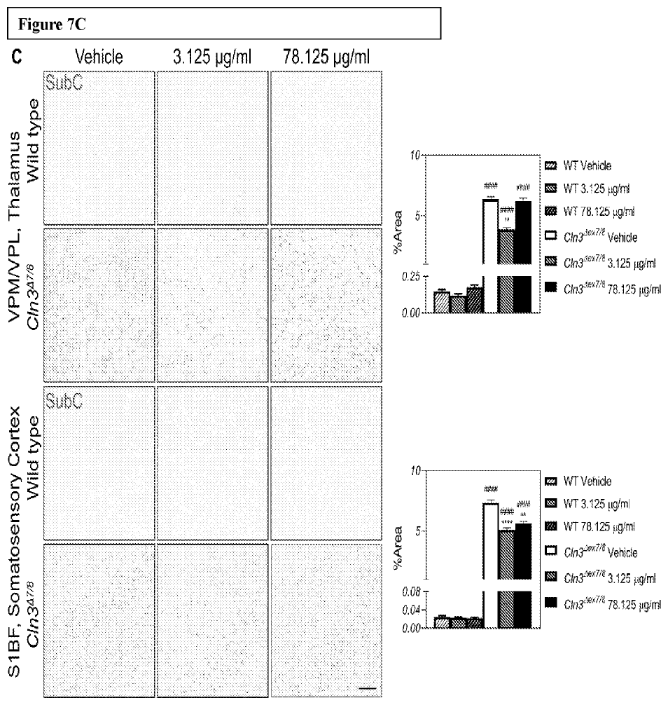

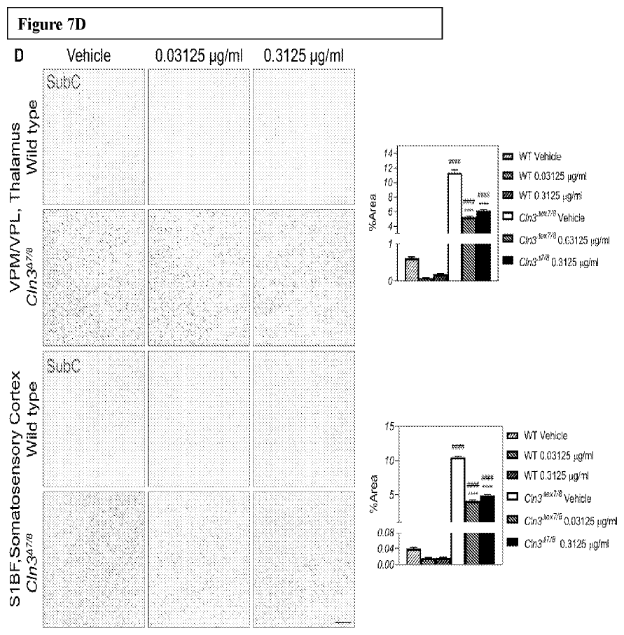

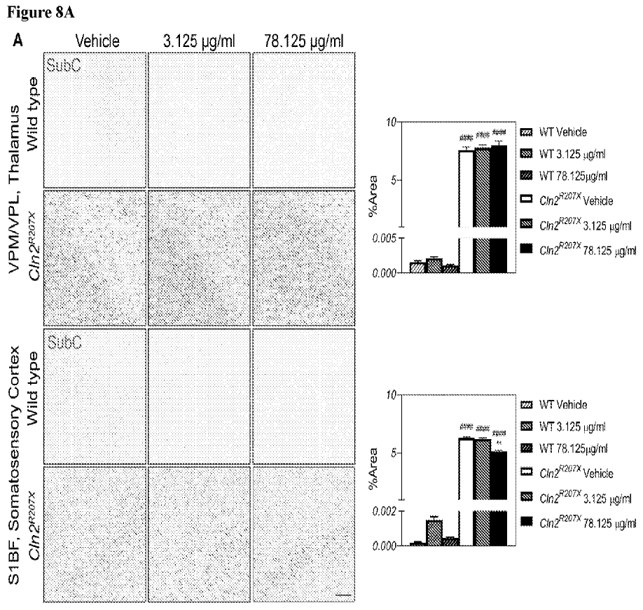

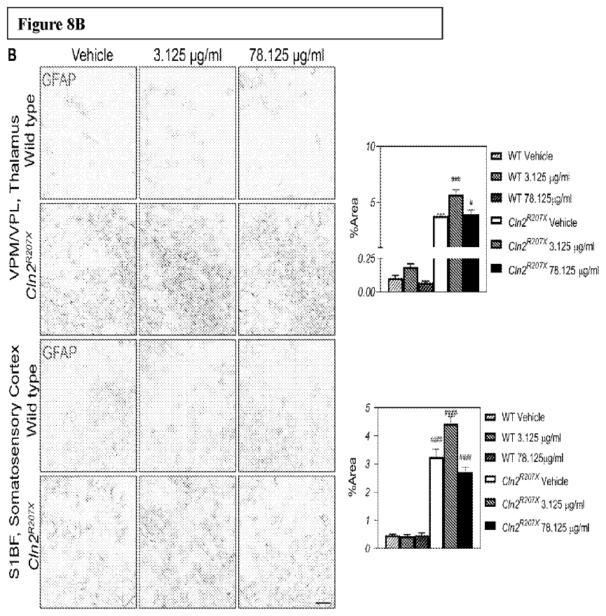

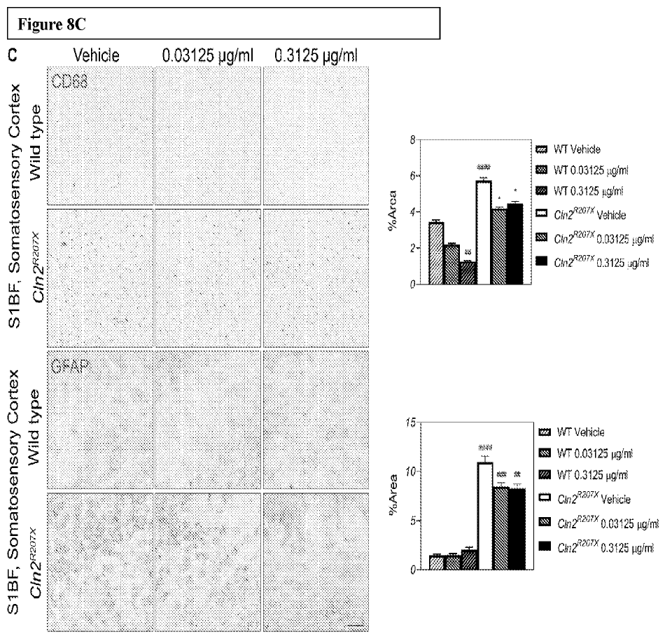

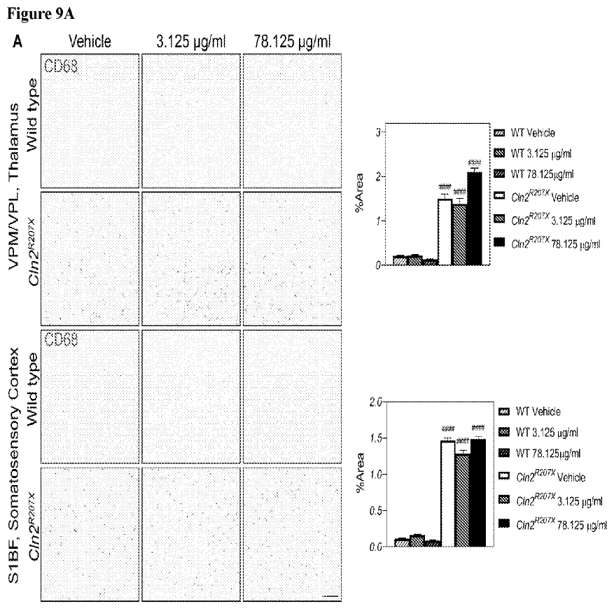

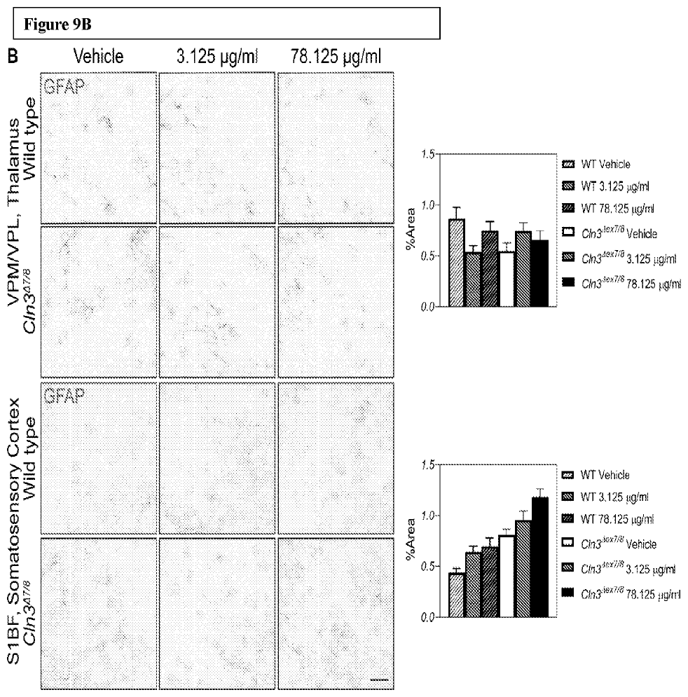

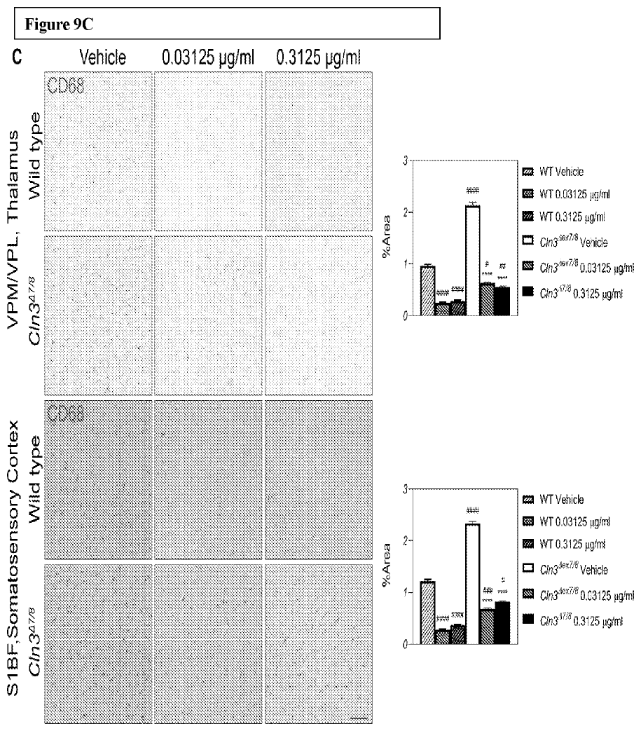

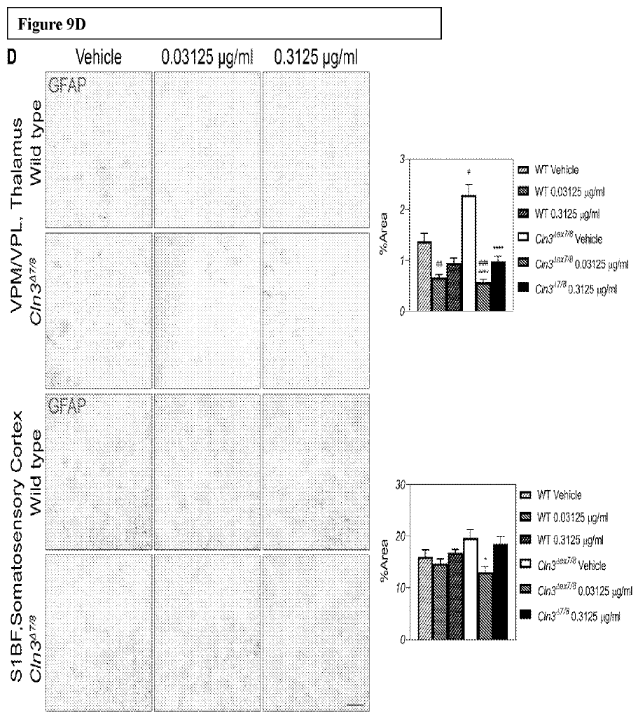

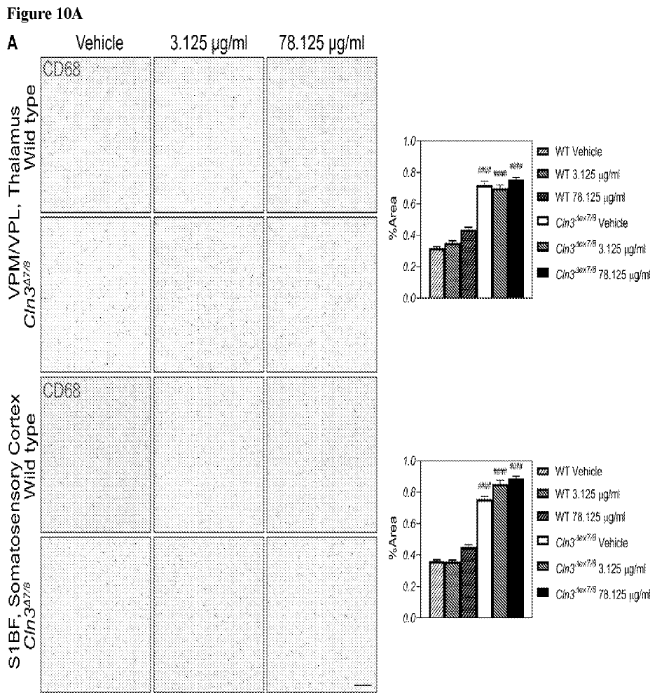

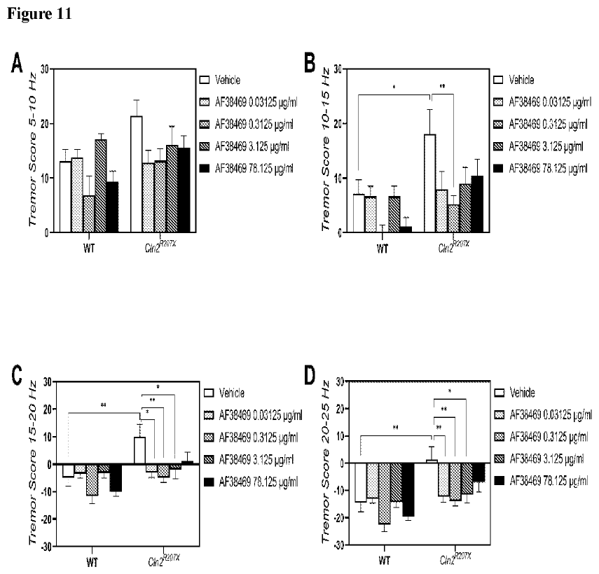

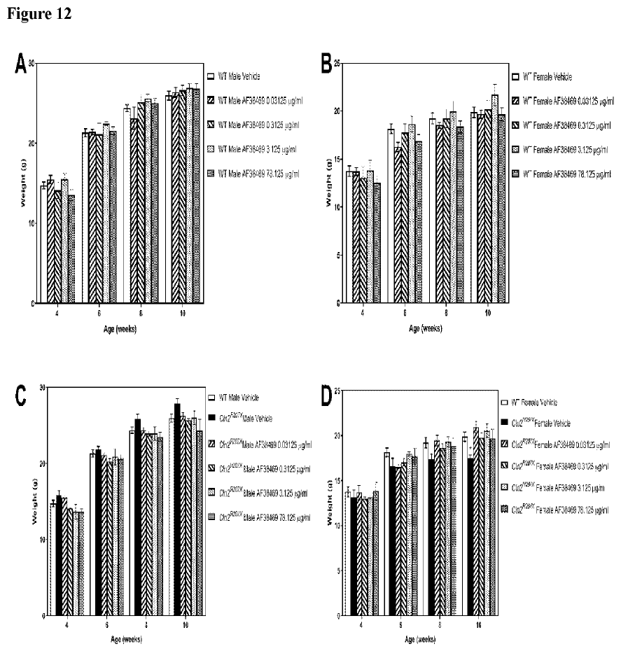

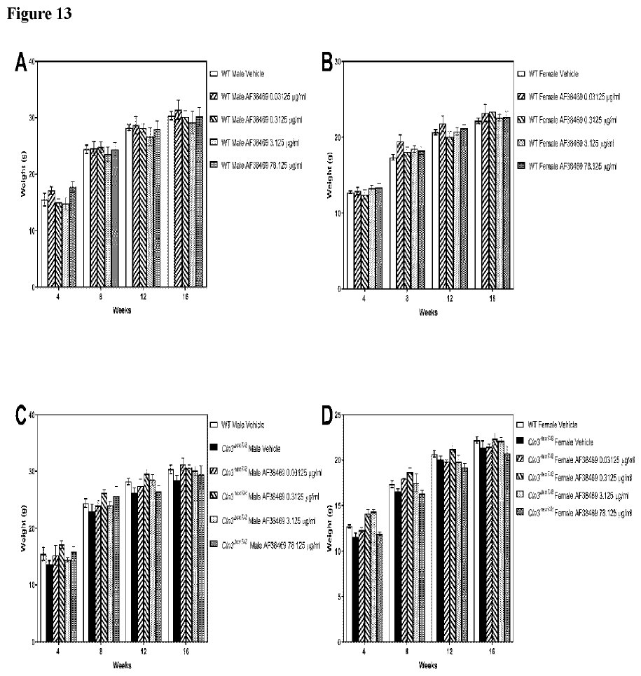

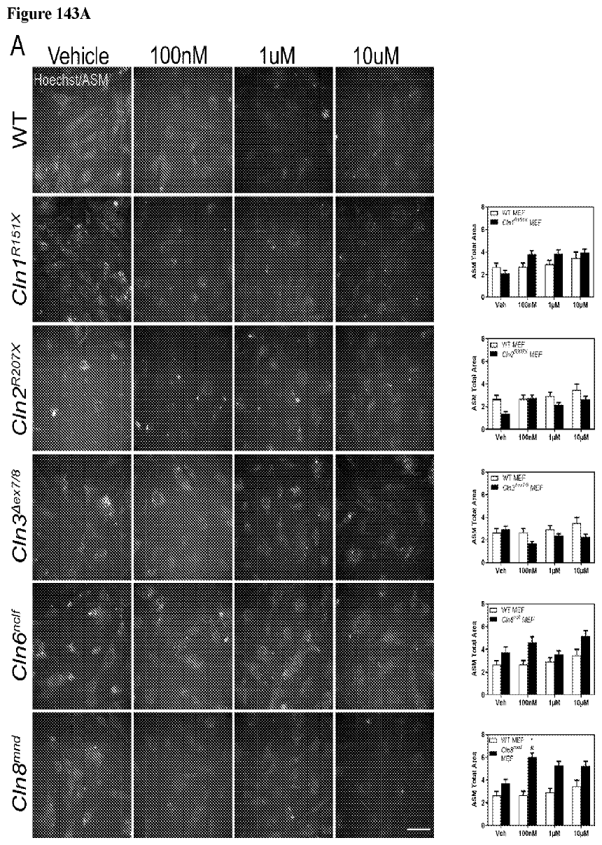

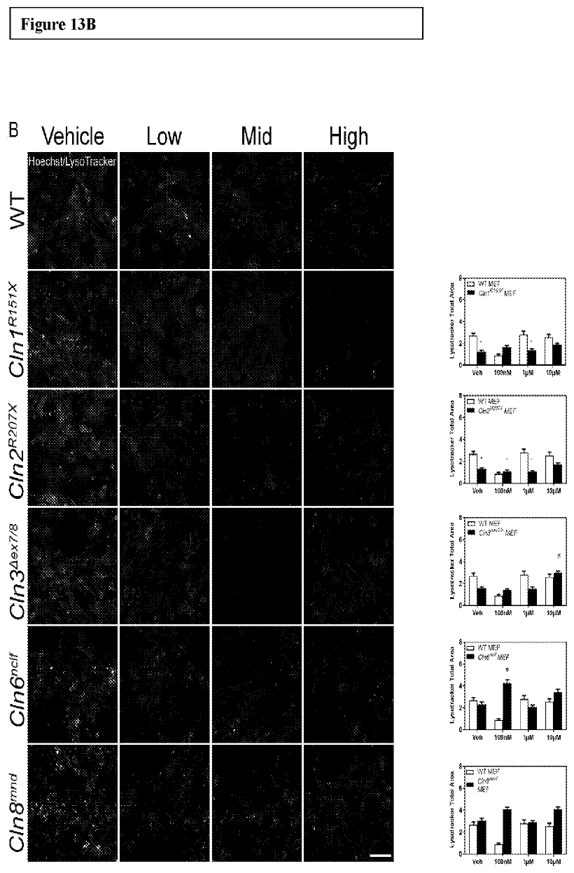

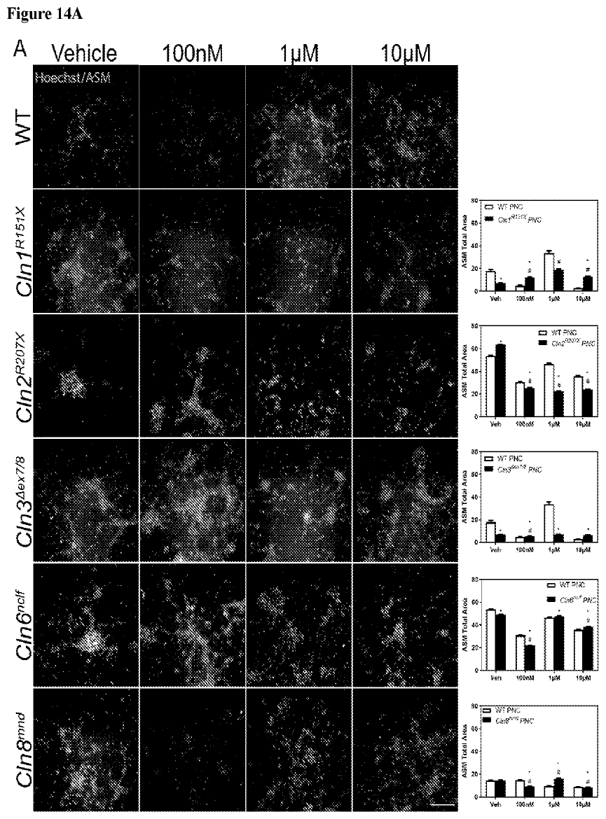

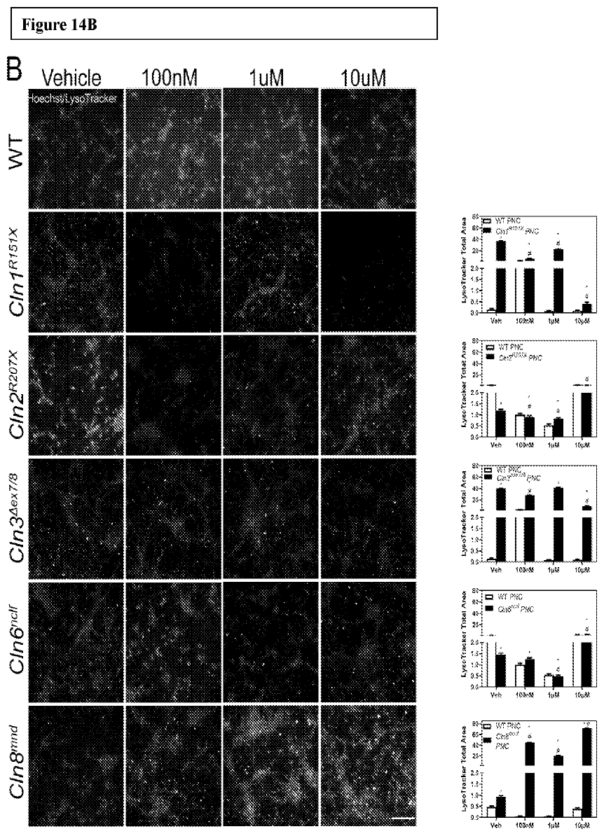

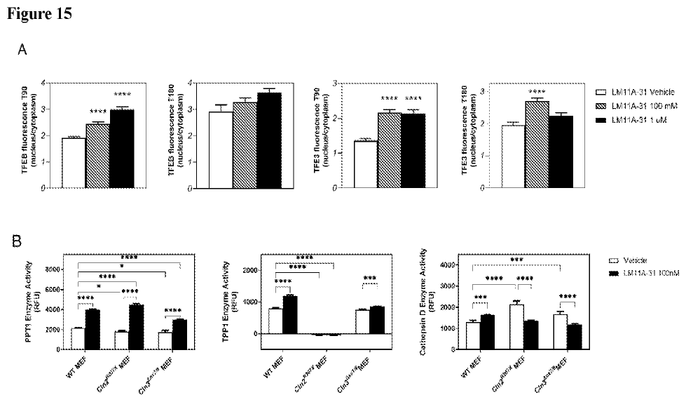

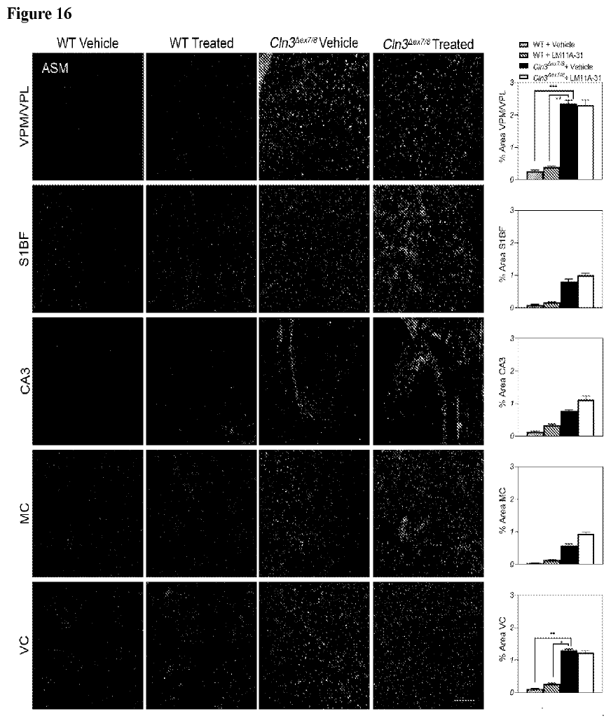

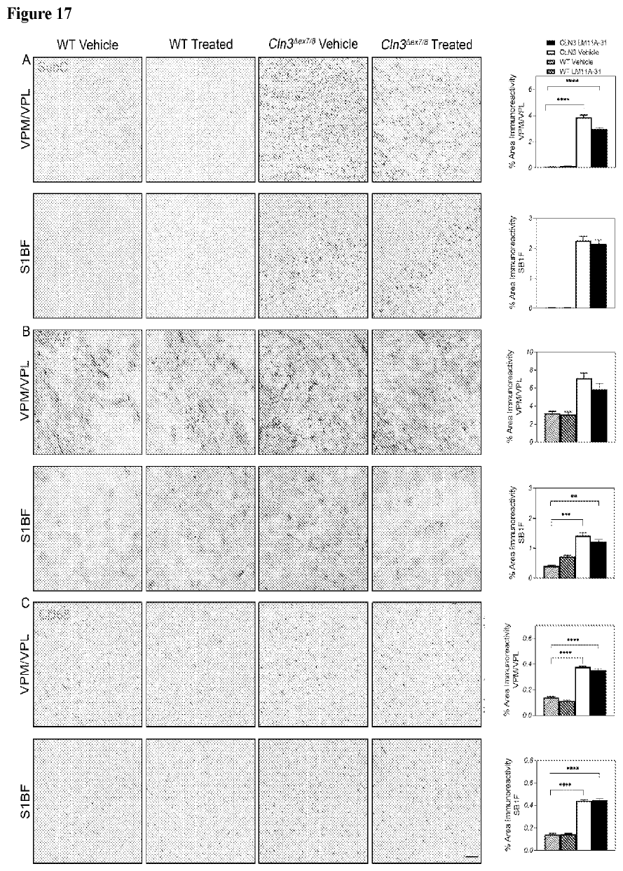

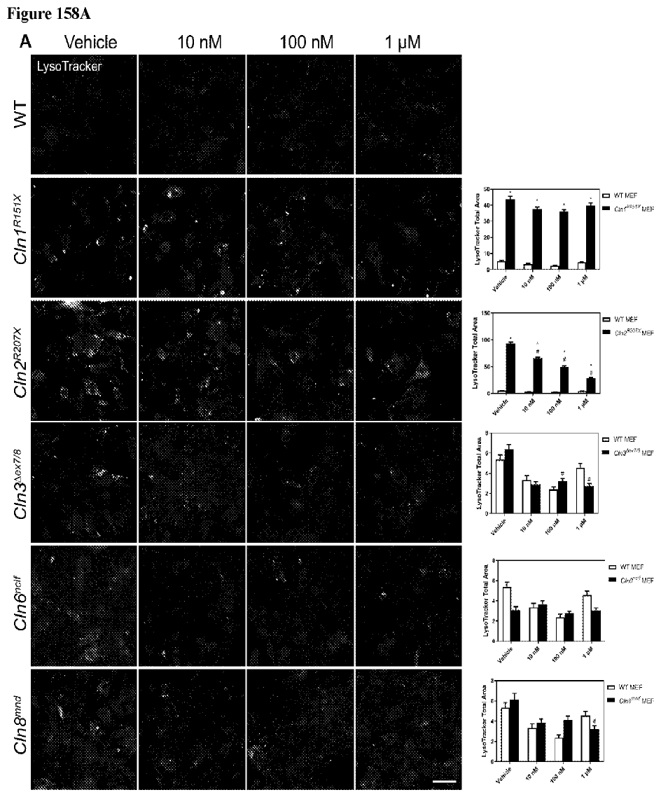

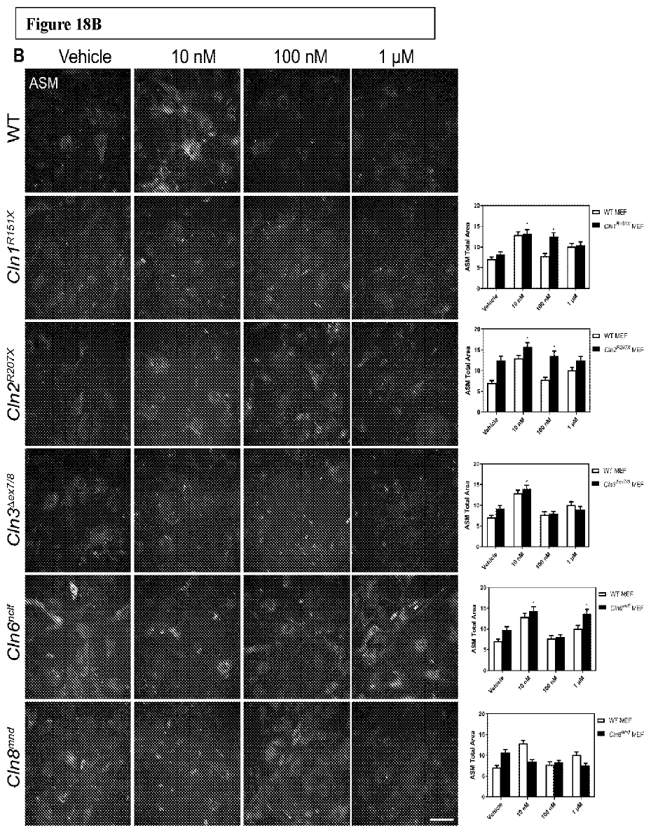

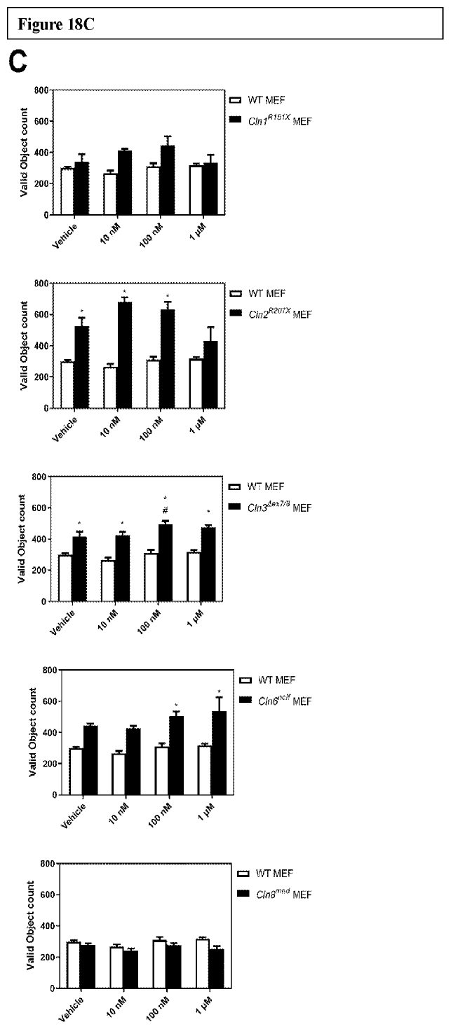

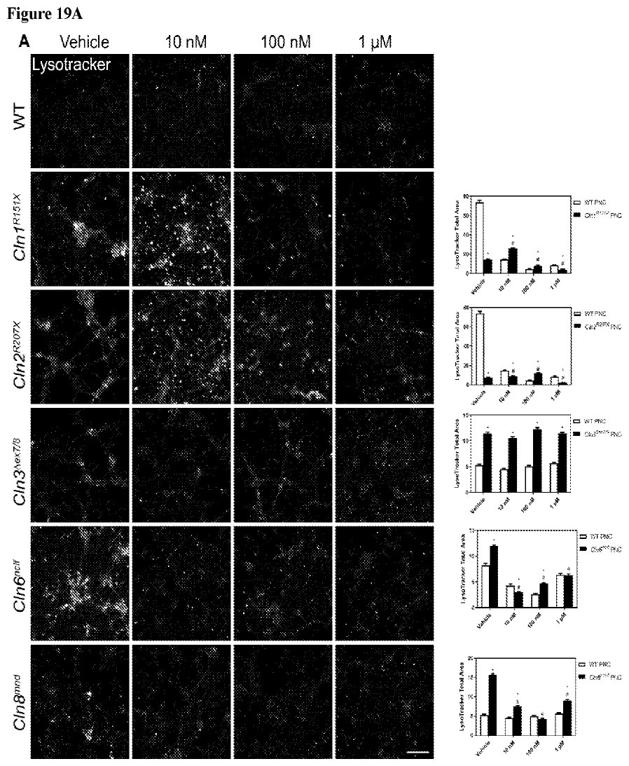

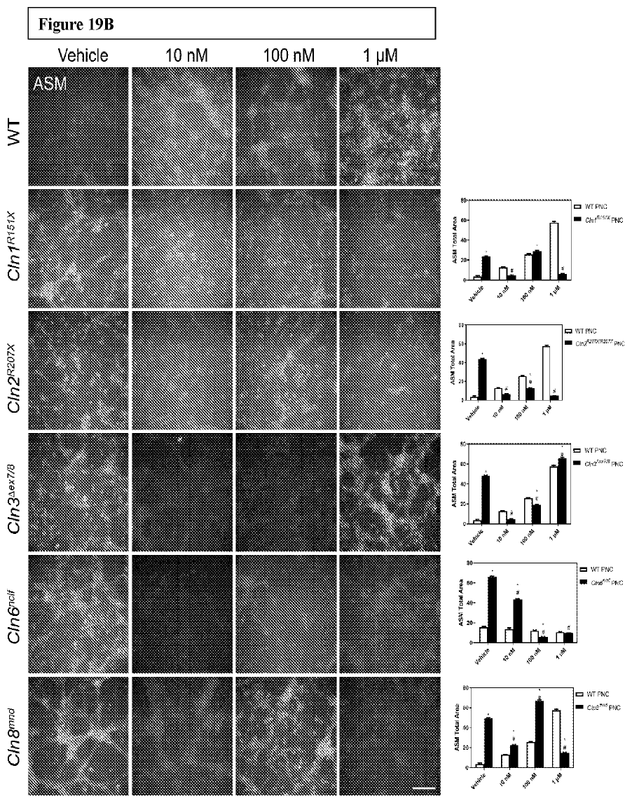

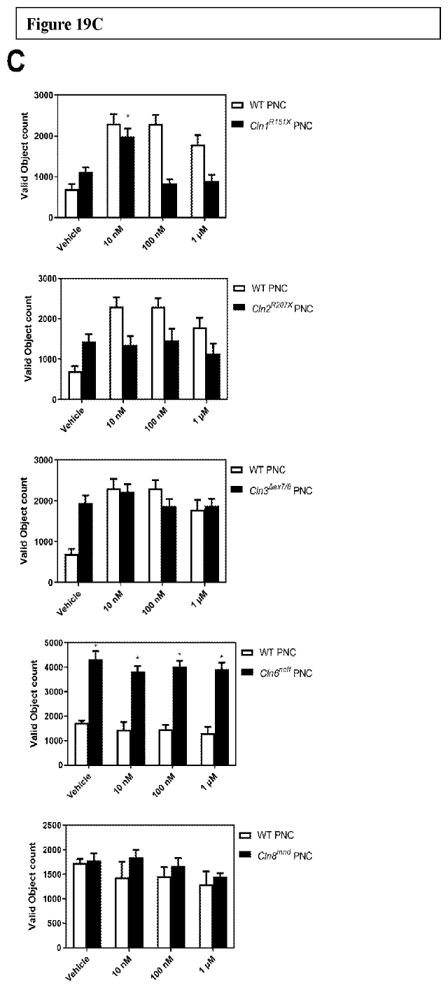

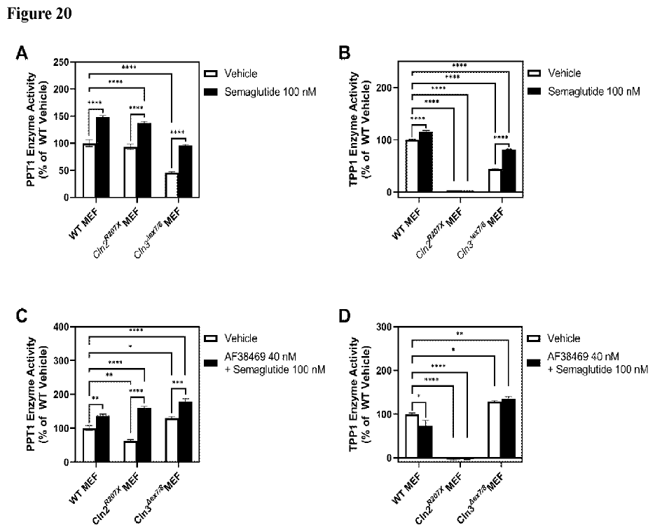

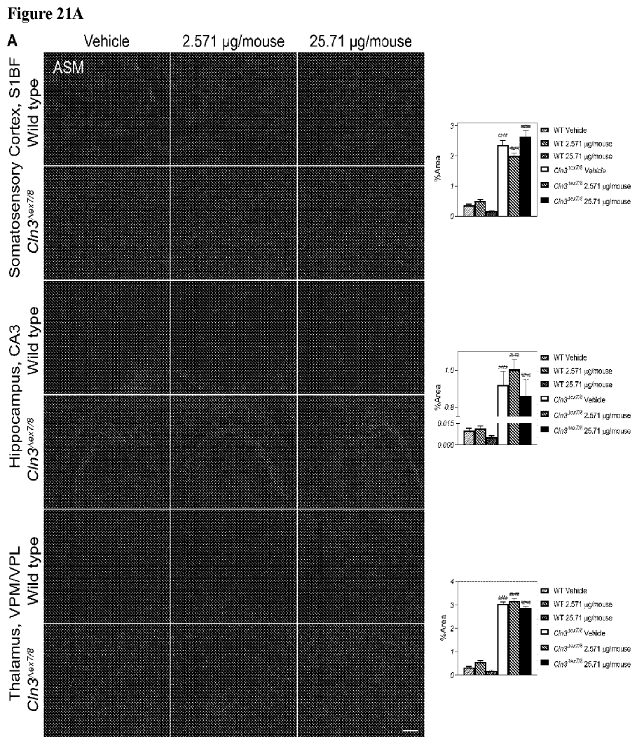

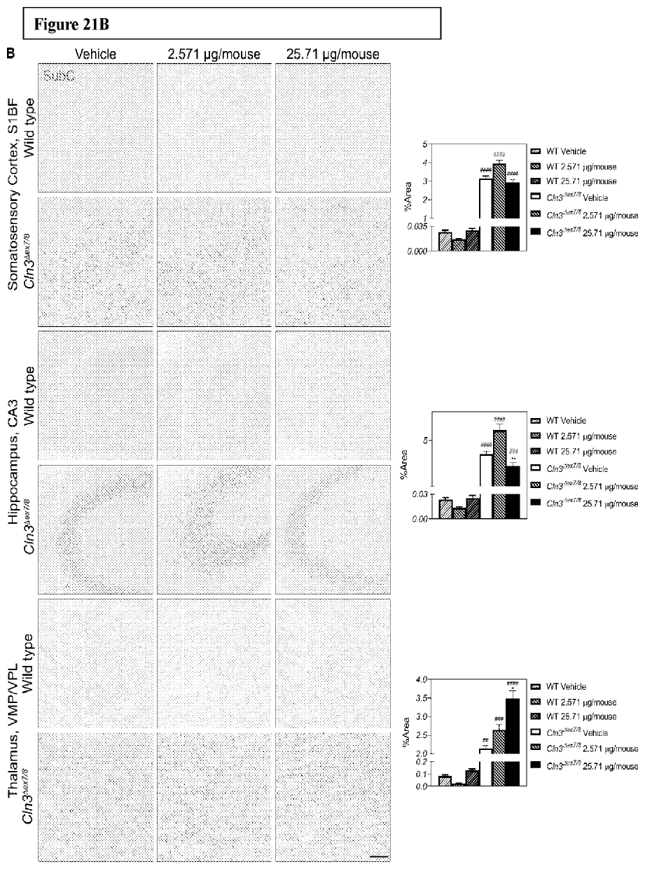

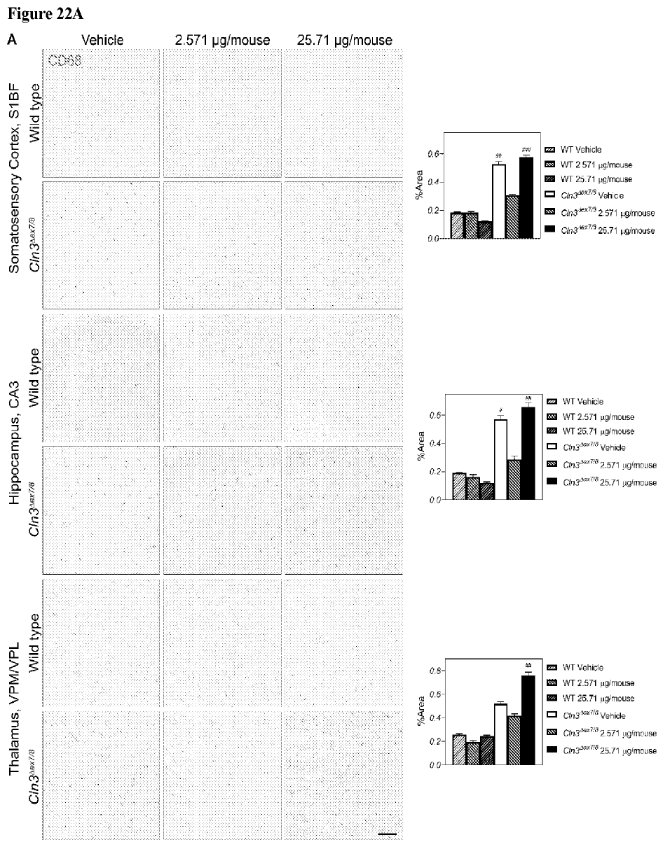

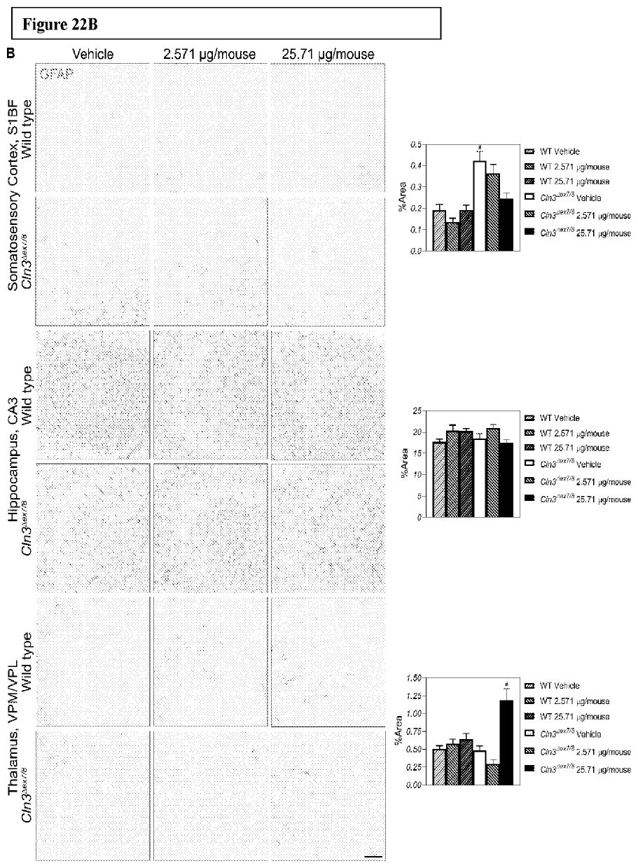

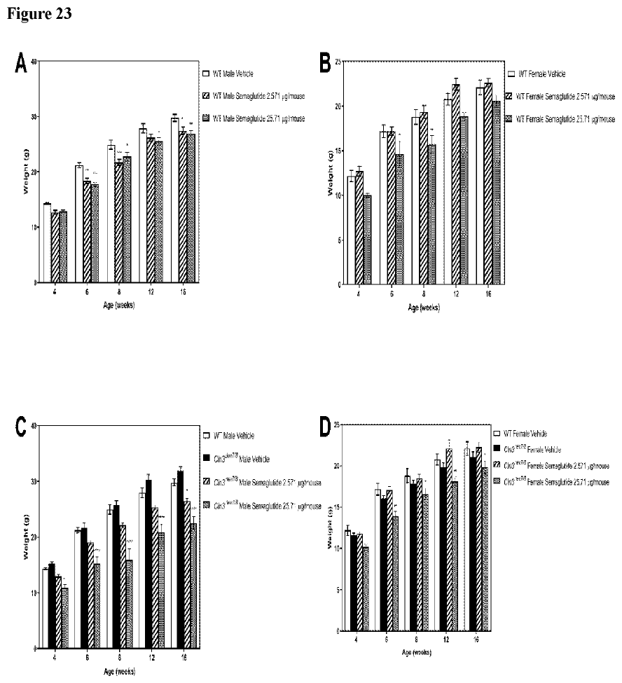

FIG. 5(A-D). Short-term chronic treatment with AF38469 reduces storage material burden in Cln2^{R207X} and Cln3^{Δex7/8} mice. (A) Treatment with AF38469 (3.125 μg/ml. 78.125 μg/ml) did not have an impact of SubC burden in the VPM/VPL of the thalamus of treated Cln2^{R207X} mice when compared to the vehicle treated Cln2^{R207X} mice. Treatment with AF38469 (78.125 μg/ml) significantly decreased Subunit C burden in the somatosensory cortex of treated Cln2^{R207X} mice when compared to the vehicle treated Cln2^{R207X} mice. n=6-8 animals/treatment group. (B) Treatment with AF38469 (0.03125 μg/ml, 0.3125 μg/ml) significantly decreased Subunit C burden in the somatosensory cortex in Cln2^{R207X} mice when compared to vehicle treated Cln2^{R207X} mice. n=6-8 animals. (C) Treatment with AF38469 (3.125 μg/ml) significantly decreased Subunit C burden in the somatosensory cortex and the VPM/VPL of the thalamus in Cln3^{Δex7/8} mice when compared to vehicle treated Cln3^{Δex7/8} mice. Treatment with AF38469 (3.125 μg/ml, 78.125 μg/ml) significantly decreased Subunit C burden in the somatosensory cortex in Cln3^{Δex7/8} mice when compared to vehicle treated Cln3^{Δex7/8} mice. n=6-8 animals/treatment group. (D) Treatment with AF38469 (0.03125 μg/ml, 0.3125 μg/ml) significantly decreased Subunit C burden in the somatosensory cortex and the VPM/VPL of the thalamus in Cln3^{Δex7/8} mice when compared to vehicle treated Cln3^{Δex7/8} mice. n=6-8 animals/treatment group. Nested one-way ANOVA. Mean±S.E.M. Sidak's multiple comparisons test. Compared to the wild type vehicle treated group. #p<0.05, ##p<0.01, ###p<0.001, ####p<0.0001. Compared to mutant vehicle treated group. *p<0.05, **p<0.01, ***p<0.001, ****p<0.0001. Scale bar=100 μm.

FIG. 6(A-C). Short-term chronic treatment with AF38469 impacts glial activation in Cln2^{R207X} mice. (A) Treatment with AF38469 (78.125 μg/ml) significantly increased the microglial activation (CD68) in the VPM/VPL of the thalamus of treated Cln2^{R207X} mice when compared to the vehicle treated Cln2^{R207X} mice. Treatment with AF38469 (3.125 μg/ml, 78.125 μg/ml) did not significantly impact the microglial activation (CD68) in the somatosensory cortex of treated Cln2^{R207X} mice when compared to the vehicle treated Cln2^{207X} mice. n=6-8 animals/treatment group. (B) Treatment with AF38469 (3.125 μg/ml. 78.125 μg/ml) did not impact astroglial activation (GFAP) in the VPM/VPL of the thalamus of treated Cln2^{R207X} mice when compared to the vehicle treated Cln2^{R207X} mice. Treatment with AF38469 (3.125 μg/ml) significantly increased the astroglial activation (GFAP) in the somatosensory cortex of treated Cln2^{R207X} mice when compared to the vehicle treated Cln2^{R207X} mice. AF38469 (78.125 μg/ml) had no impact when compared to the vehicle treated Cln2^{R207X} mice. n=6-8 animals/treatment group. (C) Treatment with AF38469 (0.03125 μg/ml, 0.3125 μg/ml) significantly and potently decreased microglial activation (CD68) in the somatosensory cortex of treated Cln2^{R207X} mice when compared to the vehicle treated Cln2^{R207X} mice whereas treatment with AF38469 at the same concentrations did not significantly impact the astroglial activation (GFAP). n=6-8 animals.

FIG. 7(A-D). Short-term chronic treatment with AF38469 impacts glial activation in Cln3^{Δex7/8} mice. (A) Treatment with AF38469 (3.125 μg/ml, 78.125 μg/ml) had no impact on microglial activation (CD68) in the VPM/VPL of the thalamus and somatosensory cortex in Cln3^{Δex7/8} mice when compared to vehicle treated Cln3^{Δex7/8} mice. n=6-8 animals/treatment group. (B) Treatment with AF38469 (3.125 μg/ml, 78.125 μg/ml) had no impact on astroglial activation (GFAP) in the VPM/VPL of the thalamus and somatosensory cortex in Cln3^{Δex7/8} mice when compared to vehicle treated Cln3^{Δex7/8} mice. n=6-8 animals/treatment group. (C) Treatment with AF38469 (0.03125 μg/ml, 0.3125 μg/ml) significantly and potently decreased microglial activation (CD68) in the VPM/VPL of the thalamus and the somatosensory cortex in Cln3^{Δex7/8} mice when compared to vehicle treated Cln3^{Δex7/8} mice. n=6-8 animals/treatment group. (D) Treatment with AF38469 (0.03125 μg/ml, 0.3125 μg/ml) significantly and potently decreased astroglial activation (GFAP) in the VPM/VPL of the thalamus in Cln3^{Δex7/8} mice when compared to vehicle treated Cln3^{Δex7/8} mice. Treatment with AF38469 (0.03125 μg/ml) significantly decreased astroglial activation (GFAP) in the somatosensory cortex in Cln3^{Δex7/8} mice when compared to vehicle treated Cln3^{Δex7/8} mice. n=6-8 animals/treatment group. Nested one-way ANOVA. Mean±S.E.M. Sidak's multiple comparisons test. Compared to the wild type vehicle treated group. #p<0.05, ##p<0.01, ###p<0.001, ####p<0.0001. Compared to mutant vehicle treated group. *p<0.05, **p<0.01, ***p<0.001, ****p<0.0001. Scale bar=100 μm.

FIG. 8(A-D) Short-term chronic treatment with AF38469 reduced tremor phenotype in Cln2^{R207X} mice. (A) Treatment with AF38469 had no impact on the 5-10 Hz tremor index score when compared to vehicle treated Cln2^{R207X} mice. (B) Treatment with AF28349 (0.3125 μg/ml) significantly reduced the tremor index score at 10-15 Hz when compared to vehicle treated Cln2^{R207X} mice. (C, D) Treatment with AF28349 (0.03125 μg/ml, 0.3125 μg/ml, 3.125 μg/ml) significantly reduced the tremor index score at 15-20 Hz and 20-25 Hz when compared to vehicle treated Cln2^{R207X} mice. n=7-8 animals/treatment group.

FIG. 9(A-D). Short-term chronic treatment with AF38469 does not impact body weight of wild type mice but rescues body weight of Cln2^{R207X} mice. (A, B) Treatment with AF38469 (0.03125 μg/ml, 0.3125 μg/ml, 3.125 μg/ml, 78.125 μg/ml) did not impact body weight of wild type male and female mice when compared to the vehicle treated wild type mice. (C, D) Treatment with AF38469 (0.03125 μg/ml, 0.3125 μg/ml, 3.125 μg/ml, 78.125 μg/ml) did not impact body weight of Cln2^{R207X} mice when compared to vehicle treated wild type mice at 4, 6. or 8 months of age. At 10 months of age, the body weights of female Cln2^{R207X} mice were reduced as compared to wild type mice; treatment with AF38469 (0.03125 μg/ml, 0.3125 μg/ml, 3.125 μg/ml, 78.125 μg/ml) normalized body weights in Cln2^{R207X} mice at this time point.

FIG. 12(A-D) Short-term chronic treatment with AF38469 does not impact body weight of wild type or Cln3^{Δex7/8} mice. (A, B) Treatment with AF38469 (0.03125 μg/ml, 0.3125 μg/ml, 3.125 μg/ml, 78.125 μg/ml) did not impact body weight of wild type male and female mice when compared to the vehicle treated wild type mice. (C, D) Treatment with AF38469 (0.03125 μg/ml, 0.3125 μg/ml. 3.125 μg/ml, 78.125 μg/ml) did not impact body weight of Cln3^{Δex7/8} mice when compared to vehicle treated wild type mice.

FIG. 13(A-B). Treatment with LM11A-31 reduces pathology in Batten disease mouse embryonic fibroblasts. (A) NCL MEFs display elevated ASM levels at DIV7. Treatment with 100 nM, 1 μM, 10 μM of LM11A-31 did not reduce the accumulation of autofluorescent storage material (ASM) in Cln1^{R151X}, Cln6^{nclf}, Cln8^{mnd}, MEFs, however a decreasing trend was seen in Cln2^{R207X} and Cln3^{Δex4/8}. n=1000-4000 cells/treatment group. (B) NCL MEFs display elevated Lysotracker™ levels at DIV7. Treatment with 100 nM, 1 μM, 10 μM of LM11A-31 significantly reduced LysoTracker™ signal in the 1 μM treatment groups of Cln1^{R151X}, Cln2^{R207X}, and a decreasing trend was seen in Cln3^{Δex7/8}, Cln6^{nclf}, Cln8^{mnd}, MEFs. A decreasing trend was seen in the 10 μM dose of Cln1^{R151X} and Cln2^{R207X}. The 100 nM dose had no impact on Lysotracker™ signal. n=650-4500 cells/treatment group. Scale bar=100 μm. For A-B, two-way ANOVA with a Scheffe post-hoc test *=significantly (p<0.05) different from wild type (WT) vehicle. #=significantly (p<0.05) different from NCL vehicle.

FIG. 14(A-B). Treatment with LM11A-31 reduces pathology in Batten disease primary neuronal cultures (A) NCL PNCs display elevated ASM levels at DIV7. Treatment with LM11A-31 significantly reduced the accumulation of autofluorescent storage material (ASM) in Cln1^{R151X} treated with 1 μM of LM11A-31, all treatment groups of Cln2^{R207X}, 1 μM of LM11A-31 treated Cln3^{Δex7/8}, and 100 nM of LM11A-31 Cln6^{nclf} and Cln8^{mnd} PNCs. n=4000-20000 cells/treatment group. (B) NCL PNCs display elevated Lyostracker™ levels at DIV7. Treatment with 100 nM of LM11A-31 significantly reduced LysoTracker™ signal in Cln2^{R207X} PNCs, and 1 μM LM11A-31 treated Cln6^{nclf} PNCs. n=5000-20000 cells/treatment group. Scale bar=100 μm. For A-B, two-way ANOVA with a Scheffe post-hoc test *=significantly (p<0.05) different from wild type (WT) vehicle. #=significantly (p<0.05) different from NCL vehicle.

FIG. 15(A-B). Treatment with LM11A-31 stimulates TFEB nuclear translocation as monitored via time course study. For (A), wild type neuro 2A rat neuroblastoma (N2A) cells were plated and transfected with a pEGFP-N1-TFEB plasmid for a 24-hour incubation. Cells were dyed and imaged on the CellInsight™ CX7 High-Content Screening Platform (CX7) then treated with media containing vehicle or LM11A-31 (100 nM, 1 μM). Cells were imaged every 30 minutes for three hours on the CX7 with incubation between imaging sessions, and results were quantified. The statistics were analyzed using Graphpad Prism™. One-way ANOVA. Mean S.E.M. Dunnett's multiple comparisons test compared to the vehicle treated group. *p<0.05, **p<0.01, ***p<0.001, ****p<0.0001. Quantification of TFEB nuclear translocation after treatment with vehicle or LM11A-31 (100 nM, 1 μM). After 90 minutes of incubation with 100 nM of LM11A-31, there was a significant increase in TFEB nuclear translocation. After 180 minutes of incubation with 100 nM and 1 μM of LM11A-31, the increasing trend continued in TFEB nuclear translocation. n=5000-6500 cells/treatment. (B) Since LM11A-31 increases the expression of lysosomal genes including the PPT1 and TPP1, and Cathepsin D enzymes, we asked whether LM11A-31 increases the overall activity levels of these enzymes in cellulo. Wild type (WT) and mutant cells were treated with vehicle or LM11A-31 (100 nM). Cells were plated on day 0 and dosed with drug-containing media on DIV3 and DIV5. Cells were lysed, and protein quantification and enzyme activity assay were performed on DIV7. Data was analyzed with GraphPad Prism™. Two-way ANOVA. Mean S.E.M. Tukey's multiple comparisons test. *p<0.05, **p<0.01, ***p<0.001. ****p<0.0001.

FIG. 16. Effects of treatment with LM11A-31 on auto florescent storage material. Beginning at wean, wild type and Cln3^{Δex7/8} mice were treated with vehicle (DI water) or LM11A-31 (0.6 mg/ml; 1.92 mg/mouse/day, targeted dose of ˜75 mg/kg) continuously through the drinking water continuing until sacrifice at 16 weeks. Upon sacrifice, one hemisphere of brain was sectioned at 50 microns on a vibratome; DAB staining immunohistochemistry was then performed using anti-GFAP (Dako, Z0334; 1:5000), anti-CD68 (AbD Serotec, MCA1957; 1:2000), anti-rabbit biotinylated (Vector Labs. BA-1000 1:1000), and anti-rat (Vector Labs, BA-9400 1:1000). Sections were imaged and analyzed using an Aperio Digital Pathology Slide Scanner (AT2) and associated software. The flash-frozen brain was sectioned into 16 μm slices and placed on slides. Slides were post-fixed for 20 minutes in chilled 10% neutral buffered formalin (NBF) and underwent serial ethanol dehydration. To quantify accumulation of autofluorescent storage material, DAPI was applied to slides and coverslipped with aqueous mounting media (Dako Faramount, Agilent, S302580-2). Sections were imaged using a Nikon NiE microscope and associated software. Treatment with LM11A-31 (0.6 mg/mL) did not decrease auto florescent storage material in the S1BF, CA3, or motor cortex in comparison to vehicle controls. A slight decrease was seen in the VPM/VPL and visual cortex compared to Cln3^{Δex7/8} untreated mice. n=8 animals. Nested one-way ANOVA. Mean±S.E.M. Sidak's multiple comparisons test. Compared to the wild type vehicle treated group. #p<0.05, ##p<0.01, ###p<0.001, ####p<0.0001. Compared to mutant vehicle treated group. *p<0.05, **p<0.01, ***p<0.001, ****p<0.0001. Scale bar=100 μm.

FIG. 17(A-C). Treatment with LM11A-31 impacts SubC accumulation and glial activation. (A) Treatment with LM11A-31 (0.6 mg/mL) showed a decreasing trend of SubC burden in the VPM/VPL of the thalamus of treated Cln3^{Δex7/8} mice when compared to the vehicle treated Cln3^{Δex7/8} mice. Treatment with LM11A-31 (0.6 mg/mL) did not significantly decreased Subunit C burden in the somatosensory cortex of treated Cln3^{Δex7/8} mice when compared to the vehicle treated Cln3^{Δex7/8} mice. n=8 animals/treatment group. Scale bar=100 μm. SubC is a major constituent of the autofluorescent storage material in NCLs (Palmer, 1992). (B) Treatment with LM11A-31 (0.6 mg/mL) had no significant decrease on astroglial activation (GFAP) in the VPM/VPL of the thalamus and somatosensory cortex but showed a decreasing trend in Cln3^{Δex7/8} mice when compared to vehicle treated Cln3^{Δex7/8} mice. n=8 animals/treatment group. (C) Treatment with LM11A-31 (0.6 mg/mL) had no impact on microglial activation (CD68) in the VPM/VPL of the thalamus and the somatosensory cortex in Cln3^{Δex7/8} mice when compared to vehicle treated Cln3^{Δex7/8} mice. n=7-8 animals/treatment group. Nested one-way ANOVA. Mean S.E.M. Sidak's multiple comparisons test. Compared to the wild type vehicle treated group. #p<0.05, ##p<0.01, ###p<0.001, ####p<0.0001. Compared to mutant vehicle treated group. *p<0.05, **p<0.01, ***p<0.001, ****p<0.0001. Scale bar=100 μm.

FIG. 108(A-C). Treatment with semaglutide impacts pathology in Batten disease mouse embryonic fibroblasts. Wild type (WT) and mutant cells were treated with vehicle or semaglutide (10 nM, 100 nM, 1 μM). Cells were plated on in vitro day 0 and dosed with drug-containing media on DIV3 and DIV5. Analysis occurred on DIV7. Concentrations of drug are shown on the x-axis above each image column; genotypes are shown to the left of each row. The y-axis of the bar graphs shows “Total Area” units which indicate % of the total cell area. The y-axis of the bar graphs in (C) shows “Valid Object Count” which reflects number of cells. Increasing concentrations of semaglutide are shown along the x-axis. Two-way ANOVA with a Scheffe post-hoc test *=significantly (p<0.05) different from wild type (WT) vehicle. #=significantly (p<0.05) different from NCL vehicle. (A) NCL MEFs display elevated Lysotracker™ signal levels at DIV7. Treatment with 10 nM, 100 nM, and 1 μM semaglutide had no impact on Lysotracker™ signal in Cln1^{R151X} and Cln6^{nclf} MEFs compared to the vehicle treated NCL mutant MEFs. The Lysotracker™ signal in Cln2^{R207X} MEFs treated with semaglutide (10 nM, 100 nM, 1 μM) was significantly reduced compared to the vehicle treated Cln2^{R207X} MEFs. Treatment with semaglutide (100 nM, 1 μM) significantly reduced the signal in Cln3^{Δex7/8} MEFs while the 10 nM dose had no impact when compared to the vehicle treated Cln3^{Δex7/8} MEFs. Treatment with semaglutide (1 μM) significantly reduced the signal in the Cln8^{mnd} MEFs while the 10 nM and 100 nM doses had no impact when compared to the vehicle treated Cln8^{mnd} MEFs. n=750-2000 cells per treatment. (B) NCL MEFs display elevated ASM levels at DIV7. Treatment with semaglutide (10 nM, 100 nM, 1 μM) had no impact on the accumulation of autofluorescent storage material (ASM) in Cln1^{R151X}, Cln2^{R207X}, Cln3^{Δex7/8}, Cln6^{nclf}, and Cln8^{mnd} MEFs. n=600-2000 cells per treatment. (C) Treatment with semaglutide (10 nM, 100 nM, 1 μM) did not significantly affect the viability of Cln1^{R151X}, Cln2^{R207X}, Cln6^{nclf}, and Cln8^{mnd} MEFs. Treatment with 100 nM of semaglutide significantly increased viability in Cln3^{Δex7/8} MEFs while doses of 10 nM and 1 μM had no impact on viability when compared to the vehicle treated Cln3^{Δex7/8} MEFs. n=9 wells per treatment. For A-C, two-way ANOVA with a Scheffe post-hoc test *=significantly (p<0.05) different from wild type (WT) vehicle. #=significantly (p<0.05) different from NCL vehicle.

FIG. 19(A-C). Treatment with semaglutide impacts pathology in Batten disease primary neuronal cultures. Wild type (WT) and mutant cells were treated with vehicle or semaglutide (10 nM, 100 nM, 1 μM). Cells were plated on in vitro day 0 and dosed with drug-containing media on DIV3 and DIV5. Analysis occurred on DIV7. Concentrations of drug are shown on the x-axis above each image column; genotypes are shown to the left of each row. The y-axis of the bar graphs shows “Total Area” units which indicate % of the total cell area. The y-axis of the bar graphs in (C) shows “Valid Object Count” which reflects number of cells. Increasing concentrations of semaglutide are shown along the x-axis. Two-way ANOVA with a Scheffe post-hoc test *=significantly (p<0.05) different from wild type (WT) vehicle. #=significantly (p<0.05) different from NCL vehicle. (A) NCLs PNCs display elevated Lysotracker™ signal levels at DIV7. Treatment with semaglutide (10 nM, 100 nM, 1 μM) significantly reduced signal in Cln6^{nclf} and Cln8^{mnd} PNCs and had no impact on Cln3^{Δex7/8} PNCs when compared to the vehicle treated NCL mutant PNCs. Treatment with semaglutide (100 nM, 1 μM) significantly reduced signal while the 10 nM dose significantly elevated signal in Cln1^{R151X} PNCs when compared to the vehicle treated Cln1^{R151X} PNCs. Treatment with semaglutide (10 nM, 1 μM) significantly reduced signal while the 100 nM dose significantly elevated signal in Cln2^{R207X} PNCs when compared to the vehicle treated Cln2^{R207X} PNCs. n=1600-7500 cells per treatment. (B) NCLs PNCs display elevated autofluorescent storage material (ASM) levels at DIV7. Treatment with semaglutide (10 nM, 100 nM. 1 μM) significantly reduced signal in Cln2^{R207X} and Cln6^{nclf} PNCs when compared to vehicle treated NCL mutant PNCs. Treatment with semaglutide (10 nM. 1 μM) significantly reduced ASM levels while the 100 nM dose had no impact on ASM levels in Cln1^{R151X} PNCs when compared to vehicle treated Cln1^{R151X} PNCs. Treatment with semaglutide (10 nM, 1 μM) significantly reduced ASM levels while the 100 nM dose significantly elevated ASM levels in Cln8^{mnd} PNCs when compared to vehicle treated Cln8^{mnd} PNCs. Treatment with semaglutide (10 nM, 100 nM) significantly reduced ASM levels while the 1 μM dose significantly elevated ASM levels in Cln3^{Δex7/8} PNCs when compared to vehicle treated Cln3^{Δex7/8} PNCs. n=1000-7000 cells per treatment. (C) Treatment with semaglutide (10 nM, 100 nM, 1 μM) did not significantly affect the viability of Cln1^{R151X}, Cln2^{R207X}, Cln3^{Δex7/8}, Cln6^{nclf}, and Cln8^{mnd} PNCS when compared to the vehicle treated NCL mutant PNCs. n=6-12 wells per treatment. For A-C, two-way ANOVA with a Scheffe post-hoc test *=significantly (p<0.05) different from wild type (WT) vehicle. #=significantly (p<0.05) different from NCL vehicle.

FIG. 20(A-D). Treatment with semaglutide increases PPT1 and TPP1 enzyme activity in Batten disease mouse embryonic fibroblasts. Wild type (WT) and mutant cells were treated with vehicle or semaglutide (100 nM) or AF38469 (40 nM) and semaglutide (100 nM). Cells were plated on in vitro day 0 and dosed with drug-containing media on DIV3 and DIV5. Cells were lysed, and protein quantification and enzyme activity assay were performed on DIV7. Data was analyzed with GraphPad Prism™. Two-way ANOVA. Mean±S.E.M. Tukey's multiple comparisons test. *p<0.05. **p<0.01, ***p<0.001, ****p<0.0001. (A) Treatment with semaglutide (100 nM) significantly increased levels of PPT1 enzyme activity in wild type, Cln2^{R207X} and Cln3^{Δex7/8} MEFs when compared to the vehicle treated mutant control. n=3 wells per treatment. (B) Treatment with semaglutide (100 nM) significantly increased levels of TPP1 enzyme activity in wild type, Cln3^{Δex7/8} MEFs when compared to the vehicle treated mutant control. Levels of TPP1 enzyme activity in Cln2^{R207X} were not altered by treatment of semaglutide. n=3 wells per treatment. (C) Treatment with AF38469 (40 nM) and semaglutide (100 nM) significantly increased levels of PPT1 enzyme activity in wild type, Cln2^{R207X} and Cln3^{Δex7/8} MEFs when compared to the vehicle treated mutant control. n=4 wells per treatment. The increases in enzyme activity observed with the combination of AF38469 and semaglutide exceeded those observed with semaglutide on its own, demonstrating a synergistic effect. (D) Treatment with AF38469 (40 nM) and semaglutide (100 nM) significantly increased levels of TPP1 enzyme activity in wild type, Cln3^{Δex7/8} MEFs when compared to the vehicle treated mutant control. Levels of TPP1 enzyme activity in Cln2^{R207X} were not altered by treatment of AF38469 and semaglutide. n=5 wells per treatment.

FIG. 21(A-B). Short-term chronic treatment with semaglutide impacts storage material burden in Cln3^{Δex7/8} mice. Beginning at wean, wild type and Cln3^{Δex7/8} mice were treated with vehicle or semaglutide 2.571 μg/mouse, 25.71 μg/mouse) through subcutaneous injections three times a week until sacrifice at 16 weeks. Upon sacrifice, brains were placed in a sagittal brain block and sliced at the midline and 3 mm right of the midline. The 3 mm sagittal piece was flash frozen with −50° C. isopentane and then sectioned on a cryostat at 16 m and placed on slides. For ASM measurements, slides were counterstained with DAPI and imaged on a Nikon ECLIPSE™ Ni-E upright microscope with a CoolSNAP™ DYNO camera. Images were extracted from S1BF of the somatosensory cortex, CA3 of the hippocampus, and the VPM/VPL of the thalamus, with multiple images taken of multiple tissues from each animal. The other hemisphere of brain was sectioned at 50 microns on a vibratome; DAB staining immunohistochemistry was then performed using anti-SubC (Abcam, ab181243) anti-rabbit biotinylated (Vector Labs, BA-1000). Sections were imaged and analyzed using an Aperio Digital Pathology Slide Scanner (AT2) and associated software. Images were extracted from S1BF of the somatosensory cortex, the CA3 of the hippocampus, and the VPM/VPL of the thalamus, key brain regions for Batten disease pathology. Percent area of immunoreactivity were quantified using a threshold analysis in ImageJ™. Statistical analyses were performed using GraphPad Prism™ (v8.4.3; San Diego, CA). The y-axis of the bar graphs shows “% Area” units which indicate % of the total cell area. Increasing concentrations of semaglutide and corresponding genotypes are shown along the x-axis. Nested one-way ANOVA. Mean S.E.M. Sidak's multiple comparisons test. Compared to the wild type vehicle treated group #p<0.05, ##p<0.01, ###p<0.001, ####p<0.0001. Compared to mutant vehicle treated group *p<0.05, **p<0.01, ***p<0.001, ****p<0.0001. (A) Treatment with semaglutide (2.571 μg/mouse, 25.71 μg/mouse) had no significant effect on the accumulation of autofluorescent storage material (ASM) in S1BF of the somatosensory cortex, the CA3 of the hippocampus, and the VPM/VPL of the thalamus of treated Cln3^{Δex7/8} mice when compared to the vehicle treated Cln3^{Δex7/8} mice. n=6-8 animals per treatment. (B) Treatment with semaglutide (2.571 μg/mouse, 25.71 μg/mouse) had no significant effect on the accumulation of mitochondrial ATP synthase subunit C (SubC) in S1BF of the somatosensory cortex of treated Cln3^{Δex7/8} mice when compared to the vehicle treated Cln3^{Δex7/8} mice. Treatment with semaglutide (25.71 μg/mouse) significantly reduced the SubC burden while the 2.571 μg/mouse dose had no significant effect in the CA3 of the hippocampus of treated Cln3^{Δex7/8} mice when compared to the vehicle treated Cln3^{Δex7/8} mice. Treatment with semaglutide (25.71 μg/mouse) significantly elevated the SubC burden while the 2.571 μg/mouse dose had no significant effect in the VPM/VPL of the thalamus of treated Cln3^{Δex7/8} mice when compared to the vehicle treated Cln3^{Δex7/8} mice. n=3-8 animals per treatment.

FIG. 22(A-B). Short-term chronic treatment with semaglutide impacts glial activation in Cln3^{Δex7/8} mice. Beginning at wean, wild type and Cln3^{Δex7/8} mice were treated with vehicle or semaglutide 2.571 μg/mouse, 25.71 μg/mouse) through subcutaneous injections three times a week until sacrifice at 16 weeks. Upon sacrifice, a hemisphere of the brain was sectioned at 50 microns on a vibratome; DAB staining immunohistochemistry was then performed using anti-GFAP (Dako, Z0334; 1:5000), anti-CD68 (AbD Serotec, MCA1957; 1:2000), anti-rabbit biotinylated (Vector Labs, BA-1000 1:1000), and anti-rat (Vector Labs, BA-9400 1:1000). Sections were imaged and analyzed using an Aperio Digital Pathology Slide Scanner (AT2) and associated software. Images were extracted from S1BF of the somatosensory cortex, the CA3 of the hippocampus, and the VPM/VPL of the thalamus, key brain regions for Batten disease pathology. Percent area of immunoreactivity were quantified using a threshold analysis in ImageJ™. Statistical analyses were performed using GraphPad Prism™ (v8.4.3; San Diego, CA). The y-axis of the bar graphs shows “% Area” units which indicate % of the total cell area. Increasing concentrations of semaglutide and corresponding genotypes are shown along the x-axis. Nested one-way ANOVA. Mean±S.E.M. Sidak's multiple comparisons test. Compared to the wild type vehicle treated group #p<0.05, ##p<0.01, ###p<0.001, ####p<0.0001. Compared to mutant vehicle treated group *p<0.05, **p<0.01, ***p<0.001, ****p<0.0001. (A) Treatment with semaglutide (2.571 μg/mouse, 25.71 μg/mouse) had no significant effect on the accumulation of microgliosis (CD68) in the S1BF of the somatosensory cortex, the CA3 of the hippocampus, and the VPM/VPL of the thalamus of treated Cln3^{Δex7/8} mice when compared to the vehicle treated Cln3^{Δex7/8} mice. n=6-8 animals per treatment. (B) Treatment with semaglutide (2.571 μg/mouse, 25.71 μg/mouse) had no significant effect on the accumulation of astrocytosis (GFAP) in the S1BF of the somatosensory cortex, the CA3 of the hippocampus, and the VPM/VPL of the thalamus of treated Cln3^{Δex7/8} mice when compared to the vehicle treated Cln3^{Δex7/8} mice. n=3-8 animals per treatment.

FIG. 23(A-D). Short-term chronic treatment with semaglutide impacts body weight of wild type and Cln3^{Δex7/8} mice. Beginning at wean, wild type and Cln3^{Δex7/8} mice were treated with vehicle or semaglutide (2.571 μg/mouse. 25.71 μg/mouse) through the subcutaneous injections three times per week until sacrifice at 16 weeks age. Body weight was measured at 4, 6, 8, 12. and 16 weeks of age. The data was analyzed with Graphpad. Two-way ANOVA. Mean±S.E.M. Dunnett's multiple comparisons test. *p<0.05, **p<0.01, ***p<0.001, ****p<0.0001. (A) Treatment with semaglutide (2.571 μg/mouse) significantly reduced the weight of the treated wild type male mice at 6, 8 and 16 weeks of age while the 25.71 μg/mouse dose significantly reduced the weight of the treated wild type male mice at 6, 8, 12, and 16 weeks of age when compared to the vehicle treated wild type male mice. n=4. (B) Treatment with semaglutide (25.71 μg/mouse) significantly reduced the weight of the treated wild type female mice at 6 and 8 weeks of age when compared to the vehicle treated wild type female mice. n=4. (C) Treatment with semaglutide (2.571 μg/mouse) significantly reduced the weight of the treated Cln3^{Δex7/8} male mice at 16 weeks of age while the 25.71 μg/mouse dose significantly reduced the weight of the treated Cln3^{Δex7/8} male mice at 4, 6, 8, 12, and 16 weeks of age when compared to the vehicle treated wild type male mice. n=4. (D) Treatment with semaglutide (25.71 μg/mouse) significantly reduced the weight of the treated Cln3^{Δex7/8} female mice at 6, 8, 12, and 16 weeks of age when compared to the vehicle treated wild type female mice. n=3-4.

### DETAILED DESCRIPTION

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All scientific and technical terms used in this application have meanings commonly used in the art unless otherwise specified.

All embodiments of any aspect of the disclosure can be used in combination, unless the context clearly dictates otherwise.

In a first aspect the disclosure provides methods for
(a) treating a a lysosomal storage disorder, comprising administering to a subject that has a lysosomal storage disorder thereof an amount effective of a sortilin (SORT1) inhibitor, a p75 neurotrophin receptor (NGFR) modulator, and/or a glucagon-like peptide-1 receptor (GLP-1R) agonist, to treat the lysosomal storage disorder, or
(b) limiting development of a lysosomal storage disorder, comprising administering to a subject at risk of developing a lysosomal storage disorder an amount effective of a sortilin (SORT1) inhibitor, a p75 neurotrophin receptor (NGFR) modulator, and/or a glucagon-like peptide-1 receptor (GLP-1R) agonist, to limit development of the lysosomal storage disorder.

As disclosed herein, the inventors have demonstrated that the recited therapeutic agents can be used to treat or limit development lysosomal storage disorders.

For example, the inventors discovered an unexpected and potent result of such treatment in lysosomal storage disorders—decreased accumulation of lysosomal storage substrates.

As defined herein, a “lysosomal storage disorder” is any disorder that is characterized by lysosomal dysfunction and the accumulation of cellular storage material consisting of macromolecular substrates. In some embodiments, the lysosomal storage disorder is an inherited/genetic disorder.

The methods can be used to treat or limit development of any lysosomal storage disorder. In one embodiment, the lysosomal storage disorder is a lysosomal disorder selected from the group consisting of NCL/Batten Disease caused by mutations in CLN gene CLN1 (PPT1), CLN2 (TPP1), CLN3, CLN4 (DNAJC5), CLN5, CLN6, CLN7 (MFSD8), CLN8, CLN10 (CTSD), CLN11, CLN12 (ATP13A2), CLN13 (CTSF), CLN14 (KCTD7), CLCN6, and/or SGSH; Pompe disease, Fabry disease, Gaucher disease, Niemann-Pick disease Types A, B, and C; GM1 gangliosidosis, GM2 gangliosidosis (including Sandhoff and Tay-Sachs), mucopolysachariddoses (MPS) types I (Hurler disease)/II (Hunter disease)/IIIa (Sanfilippo A)/IIIB (Sanfilippo B)/IIIc (Sanfilippo C)/IIId (Sanfilippo D)/IVA (Morquio A)/IVB/VI/VII (Sly)/IX, mucolipisosis III (I-cell) and IV, multiple sulfatase deficiency; sialidosis, galactosialidosis, α-mannosidosis, β-mannosidosis, apartylglucosaminuria, fucosidosis, Schindler disease, metachromatic leukodystrophy caused by deficiencies in either arylsulfatase A or Saposin B, globoid cell leukodystrophy (Krabbe disease), Farber lipogranulomatosis, Wolman and cholesteryl ester storage disease. pycnodystostosis, cystinosis, Salla disease, Danon disease, Griscelli disease Types 1/2/3, Hermansky Pudliak Disease, and Chédiak-Higashi syndrome.

In specific embodiments, the lysosomal storage disorder comprises NCL/Batten Disease caused by mutations in one or more genes selected from the group consisting of CLN1 (PPT1), CLN2 (TPP1), CLN3, CLN4 (DNAJC5), CLN5, CLN6, CLN7 (MFSD8), CLN8, CLN10 (CTSD), CLN11, CLN12 (ATP13A2), CLN13 (CTSF), CLN14 (KCTD7), CLCN6, and/or SGSH.

In various embodiments, the subject at risk of a lysosomal storage disorder has one or more of the following risk factors detailed below. For all NCL mutations, see the NCL Mutation Database, maintained by University College London (UCL web site ncl-disease/mutation-and-patient-database/mutation-and-patient-datasheets-human-ncl-genes. Mutations are listed according to HGMD nomenclature (HGMD web site/docs/mut_nom.html).
(A) At risk of Neuronal Ceroid Lipofuscinosis (NCL)/Batten Disease based on one or more mutations in Ceroid Lipofuscinosis Neuronal (CLN) gene CLN1 (palmitoyl protein thioesterase 1, PPT1), including but not limited to IVs2+1 G>A, c.-109C>A, c.1-83G>A, c.3G>A, c.20_47del28, c.29T>A, c.109C>A, c.114G>T, c.114G>A, c.114delG, c.117T>A, c.125-2A>G, c.124+1G>A, c.124+1215_235-102del3627, c.125-15T>G, c.125G>A, c.132_133insTGT, c.134G>A, c.163A>T, c.167-168insA, c.169dupA, c.174-175delG, c.223A>C, c.235-3T>C, c.236A>G, c.255_257delCTT, c.271-287delinsTT, c.272A>C, c.287G>A, c.310A>T, c.312delA, c.322G>C, c.325T>G, c.353G>A, c.362+61C>T, c.363-3T>G, c.363-16C>G, c.363-4G>A, c.364A>T, c.398delT, c.401C>T, c.413C>T, c.433+79A>G, c.451C>T (analogous to that present in mouse/cell model CLN1 R151X), c.455G>A, c.456C>A, c.490C>T, c.529C>G, c.533A>T. c.536+1G>A IVS5+1G>A, 536+2T>C. c.541G>T, c.541G>A, c.544C>T, c.550G>A, c.538dupC, c.558G>A, c.560A>G, c.566C>G, c.627+4A>G, c.628-1G>T, c.644delA, c.655T>C, c.656T>A, c. 674T>C, IVS7-2A>T, c.683T>G, c.722C>T, c.727-2A>T, c.739T>C, c.749G>T, c.776insA, c.866T>C, c.871C>T, c.888G>A, c.*526_*529delATCA, and/or c.914T>C;
(b) at risk of CLN1 disease based on presence of intracellular granular osmophilic deposits in tissue biopsies;
(c) at risk of Neuronal Ceroid Lipofuscinosis (NCL) based on one or more mutations in CLN2 (tripeptitdyl peptidase 1, TPP1) gene, including but not limited to c.17+1G>C, c.18-3C>G, c.37dup, c.38T>C, c.89+1G>A, c.89+2_887del, c.89+4A>G, c.89+5G>C, c.139C>G, c.163C>T, c.177-180del, c.184T>A, c.184_185del, c.196C>T, c.225A>G, c.228C>A, c.229G>A, c.229G>T, c.229G>C, c.229+3G>C, c.299A>G, c.237C>G, c.311T>A, g.3081-3091del, c.337dup, c.357dup, c.381-2A>G, c.381-1G>C, c.377_387del, c.379C>T, c.380G>A, c.380+55G>A, c.381-17 381-4del, c.381-2A>G, c.381-1G>C, c.406-409dup, c.431G>A, c.457T>C, IVS5-1G>C, IVS5-1G>A, c.481C>T, c.497dup, c.509-1G>C, c.528del, c.605C>T, c.616C>T, c.617G>A, c.617G>C, c.622C>T (analogous to that in mouse and cell models Cln2R208X), c.625T>C, c.636C>T, c.640C>T, c.646G>A, c.650G>T, c.713C>G, c.729C>G, c.731T>C, c.744dupA, c.775del, c.790C>T, c.797G>A, c.802del, c.824T>C, c.822_837del, c.827A>T, c.829G>A, c.833A>C, c.843G>T, c.851G>T, c.857A>G, c.860T>A, IVS7-18, c.887G>A, c.887-10A>G, c.877-18A>G, c.888_1066del, c.902-1080del, c.923+C>A, c.959T>G, c.969-976del, c.972_979del, c.984_986del, c.987_989delinsCTC. c.1007A>G, c.1027G>A, IVS8+2T>G, c.1015C>T, c.1016G>A, c.1027G>A, c.1029G>C, c.1048C>T, c.1049G>A, c.1052G>T, c.1057A>C, c.1058C>A, c.1062del, c.1064T>C, c.1076-2A>G, c.1076-2A>T, c.1075+2T>G, c.1075+2T>C, c.1093T>C, c.1106dup, c.1107T>C, c.1093T>C, c.1094G>A, c.1106dup, c.1108G>A, c.1107_1108del, c.1145G>A, c.1145+2T>G, c.1146C>G, c.1154T>A, c.1166G>A, c.1204G>T, c.1226G>A, c.1226G>T, c.1239_1240ins6, c.1261T>A, c.1266G>A, c.1266G>C, c.1266+1G>C, c.1266+5G>A, c.1284G>T, c.1340G>A, c.1343C>T, c.1343C>A, c.1351G>T, c.1354G>A, c.1358C>T, c.1358C>A, c.1361C>A, c.1376A>C, c.1379G>A, c.1397T>G, c.1417G>A, c.1424del, c.1424C>T, c.1425+1G>C, c.1438G>A, c.1439T>G, c.1442T>G, c.1444G>C, c.1444G>A, c.1467del, c.1471del, c.1497del, c.1501G>T, c.1510A>T, c.1525C>T, c.1547_1548insTCAT, c.1551+1G>A, c1551+5_1551+6delinsTA, c.1552-1G>C, c.1547_1548del, c.1548_1551dup, c.1551+1G>T, c.1552-1G>A, c.1593dup, c.1595dup, c.1603G>C, c.1611_1621del, c.1613C>A, c.1626G>A, c.1630C>T, c.1642T>C, c.1644G>A, g.5541C>T, IVS12-1G>C, c.1595insA, c.1663del, c.1677_1678delTC, and/or c.1678_1679del;
(d) at risk of CLN2 disease based on presence of storage material in tissue biopsies with curvilinear profiles;
(e) at risk of Neuronal Ceroid Lipofuscinosis (NCL) based on one or more mutations in ceroid lipofuscinosis neuronal 3 (CLN3) gene, including but not limited to c.-1101C>T, c.-684_-676delTGAAGC, c.1A>C, c.49G>T, c.105G>A, c.125+1G>C, c.125+5G>A, c.126-1G>A IVS2-1G>A, c.126-1G>A, c.214C>T, c.222+2T>G, c.222+5G>C, c.233_234insG, c.265C>T, c.294-58G>A, c.294-80G>A, c.302T>C, c.370dupT, c.374G>A, c.375-3C>G, c.391A>C, c.400T>C, c.424delG, c.379delC, c.461-280_677+382del (the most common NCL mutation, and analogous to CLN3 delta ex7-8 mouse and cell models), c.462-677del, c.472G>C, c.560G>C, c.565G>C, c.575G>A, c.582G>T, c.791-1056del, c.791-1056del, c.461_1413del, c.461-1G>C, c.461-3C>G, c.461-13G>C, c.482C>G, c.485C>G, c.302T>C, c.374-375insCC, c.378+379dupCC, c.424delG, IVS6-13G>C, c.482C>G, c.485C>G, c.494G>A, c.509T>C, IVS7+1G>C, c.533+1G>C, c.5331G>A, c.558_559delAG, c.565G>T, c.569delG, c.586-587insG, c.586dupG, c.586-587insG, c.597C>A, c.622-623insT, c.622dupT, c.631C>T, c.784A>T, c.790+3A>C, c.791-802_1056+1445del2815, c.816_817del, c.831G>A, c.837+5G>A, c.868G>T, c.883G>A, c.883G>T, c.906+5G>A, c.906+49del, c.917T>A, c.944dupA, c.944-945insA, c.954_962+18del27, c.963-1G>T, c.966C>G, c.979C>T, c.988G>A, c.988G>T, 1000C>T, c.1001G>A, c.1045_1050del, c.1048delC, c.1054C>T, c.1056G>C, c.1056+3A>C, c.1056+34 C>A, IVS14-1G>T, c.1135_1138delCTGT, c.1195G>T, c.1198-1G>T, c.1211A>G, c.1213C>T, c.1247A>G, c.1268C>A, and/or c.1272delG;
(f) at risk of CLN3 disease based on presence of storage material in tissue biopsies with fingerprint profiles;
(g) at risk of CLN3 disease based on presence of vacuolated lymphocytes;
(h) at risk of Neuronal Ceroid Lipofuscinosis (NCL) based on mutations in CLN4 (DNAJ homolog subfamily C member 5, DNAJC5) including c.346_348delCTC, c.344T>G, and/or c.370-399dup;
(i) at risk of CLN4 disease based on presence of intracellular granular osmophilic deposits in tissue biopsies;
(j) at risk of CLN4 disease based on presence of storage material in tissue biopsies with curvilinear profiles or fingerprint profiles;
(k) at risk of Neuronal Ceroid Lipofuscinosis (NCL) based on one or more mutations in CLN5 gene, including but not limited to c.4C>T, c.61C>T, c.72A>G, c.223T>C, c.225G>A, c.234C>G, c.291dupC, c.320+8C>T, c.320+18C>T, c.335G>A, c.335G>C, c.337G>A, c.433C>T, c.486+5G>C, c.486+139_712+2132del, c.524T>G, c.527_528insA, c.528T>G, c.565C>T, c.575A>G, c.593T>C, c.613C>T, c.619T>C, c.620G>C, c.669dupC, c.671G>A, c.694C>T, c.726C>A, c.741_747delinsTT, c.772T>G, c.835G>A, c.907_1094del188, c.919delA, c.935G>A, c.955_970del16, c.1026C>A, c.1054G>T, c.1072_1073delTT, c.1083delT, c.1103A>G, c.1103_1106delAACA, c.1121A>G, c.1137G>T, c.1175delAT, c.1175_1176celAT, and/or c.*33A>G;
(l) at risk of CLN5 disease based on presence of intracellular granular osmophilic deposits in tissue biopsies;
(m) at risk of CLN5 disease based on presence of storage material in tissue biopsies with curvilinear profiles or fingerprint profiles;
(n) at risk of Neuronal Ceroid Lipofuscinosis (NCL) based on one or more mutations in ceroid lipofuscinosis neuronal (CLN6) gene, including but not limited to c.13C>T, c.100G>A, c.139C>T, c.13C>T, c.144G>A, c.150C>G, c.184C>T, c.185G>A, c.198+2dup, c.200T>C, c.209C>T, c.214G>C, c.214G>T, c.218-220dupGGT, c.231C>G, c.244G>T, c.247G>C, c.248A>T, c.250T>A, c.251del, c.252C>G, c.268_271dup, c.270C>G, c.278C>T, c.296A>G, c.298-13C>T, c.298-6C>T, c.307C>T, c.308G>A, c.311C>T, c.316dup, c.348C>A, c.34G>A, c.363_365dup, c.368G>A, c.382C>G, c.395_396del, c.406C>T, c.426C>G, c.443T>A, c.445C>T, c.446G>A, c.461_463del, c.476C>T, c.485T>G, c.486+1G>T, c.486+8C>T, c.49G>A, c.506T>C, c.509A>G, c.510_512del, c.516T>A, c.519del, c.542+5G>T, c.552dup, c.557T>C, c.662A>C, c.662A>G, c.663C>G, c.700T>C, c.712_713delinsAC, c.715_718del, c.721A>G, c.722T>C, c.723G>T, c.727del, c.755G>A, c.768C>G, c.775G>A, c.776G>T, c.794_796del, c.7del, c.809T>C, c.829_836delinsCCT, c.889C>A, c.890del, c.892G>A, c.896C>T, c.898T>C, c.917_918dup, and/or exon 1 deletion;
(o) at risk of CLN6 disease based on presence of storage material in tissue biopsies with curvilinear profiles, fingerprint profiles or rectilinear complex;
(p) at risk of Neuronal Ceroid Lipofuscinosis (NCL) based on one or more mutations in CLN7 (major facilitator superfamily domain containing 8, MFSD8) gene, including but not limited to c.2T>C, c.63-1G>A, c.63-4del, c. 103C>T, c.154G>A, c.233G>A, c.259C>T, c.325_339del, c.362A>G, c.416G>A, c.468_469delinsCC, c.472G>A, c.479C>A, c.479C>T, c.493+3A>C, c.525T>A, c.554-1G>C, c.554-5A>G, c.588del, c.590del, c.627_643del, c.697A>G, c.754+1G>A, c.754+2T>A, c.863+1G>C, c.863+2dup, c.863+3_863+4insT, c.881C>A, c.894T>G, c.929G>A, c.1006G>C, c.1102G>C, c.1103-2del, c.1141G>T, c.1219T>C, c.1235C>T, c.1286G>A, c.1340C>T, c.1361T>C, c.1367G>A, c.1373C>A, c.1393C>T, c.1394G>A, c.1408A>G, c.1420C>T, and/or c.1444C>T;
(q) at risk of CLN7 disease based on presence of storage material in tissue biopsies with curvilinear profiles, fingerprint profiles or rectilinear complex;
(r) at risk of Neuronal Ceroid Lipofuscinosis (NCL) based on one or more mutations in ceroid lipofuscinosis neuronal 8 (CLN8) gene, including but not limited to c.1A>G, c.[46C>A; 509C>T], c.70C>G, c.88delG, c.88G>C, c.180_182delGAA, c.208C>T, c.209G>A, c.227A>G, c.320T>G, c.374A>G, c.415C>T, c.464C>T, c.470A>G, c.473A>G, c.507C>T, c.544-2566_590del2613, c.562_563delCT, c.581A>G, c.610C>T, c.611G>T, c.620T>G, c.637_639delTGG, c.661G>A, c.66delG, c.661G>A, c.677T>C, c.685C>G, c.709G>A, c.728T>C, c.763T>C, c.766C>G, c.789G>C, c.792C>G, c.806A>T, and/or del 8p23.3;
(s) at risk of CLN8 disease based on presence of storage material in tissue biopsies with curvilinear profiles, fingerprint profiles;
(t) at risk of CLN8 disease based on presence of intracellular granular osmophilic deposits in tissue biopsies;
(u) at risk of Neuronal Ceroid Lipofuscinosis (NCL) based on one or more mutations in CLN10 (cathepsin D, CTSD) gene, including but not limited to c.205G>A, c.269_269insC, c.299C>T, c.353-12C>T, c.353-17C>T, c.446G>T, c.685T>A, c.764dupA. s.827+13T>C, c.828-17G>A, c.845G>A. c.970G>A, c.1149G>C, and/or c.1196G>A;
(v) at risk of CLN10 disease based on presence of intracellular granular osmophilic deposits in tissue biopsies;
(w) at risk of Neuronal Ceroid Lipofuscinosis (NCL) based on one or more mutations in CLN11 (granulin, GRN) gene, including but not limited to c.813_816del, c.1477C>T, and/or c.900_901dupGT;
(x) at risk of CLN11 disease based on presence of storage material in tissue biopsies with fingerprint profiles;
(y) at risk of Neuronal Ceroid Lipofuscinosis (NCL) based on one or more mutations in CLN12 (ATPase cation transporting 13A2, ATP13A2) gene, including but not limited to c.2429T>G;
(z) at risk of CLN12 disease based on presence of intracellular granular osmophilic deposits in tissue biopsies;
(aa) at risk of Neuronal Ceroid Lipofuscinosis (NCL) based on one or more mutations in CLN13 (cathepsin F, CTSF) gene, including but not limited to c.213+1G>C, c.416C>A, c.691A>G, c.734G>A, c.954del, c.962A>G, c.977G>T, c.1211T>C, c.1243G>A, c.1373G>C, and/or c.1439C>T;
(bb) at risk of CLN13 disease based on presence of storage material in tissue biopsies with fingerprint profiles;
(cc) at risk of Neuronal Ceroid Lipofuscinosis (NCL) based on one or more mutations in CLN14 (potassium channel tetramerization domain containing 7, KCTD7) gene, including but not limited to c.190A>G, c.280C>TA>T, c.827A>G, c.295C>T, c.322C>A, c.335G>A, c.343G>T, c.550C>T, c.594delC, c.634C>T, c.704G>C, c.818A>T, c.827A>G, c.861_863delAT, and/or deletion of exons 3 and 4;
(dd) at risk of CLN14 disease based on presence of storage material in tissue biopsies with curvilinear profiles. fingerprint profiles, or rectilinear complex;
(ee) at risk of CLN14 disease based on presence of intracellular granular osmophilic deposits in tissue biopsies;
(ff) at risk of Neuronal Ceroid Lipofuscinosis (NCL) based on one or more mutations in chloride voltage-gated channel 6 (CLCN6) gene, including but not limited to c.1738G>A and/or c.1883C>G;
(gg) at risk of Neuronal Ceroid Lipofuscinosis (NCL) based on one or more mutations in N-sulfoglucosamine sulfohydrolase (SGSH) gene, including but not limited to c.904T>C and/or c.1075G>A;
(hh) at risk of Pompe disease based on one or more mutations in alpha glucosidase (GAA) gene, including but not limited to c.-32-13T>G. c.525delT, c.2481+102_2646+31del, c.2662G>T, c.1935C>A, c.2238G>C, c.2560C>T, c.546G>T, c.1726G>A, c.2065G>A, [c.1726G>A; c.2065G>A], c.510C>T, [c.510C>T; c.-32-13T>G] (see web site pompevariantdatabase.nl/pompe_mutations_list.php?orderby=aMut_ID1);
(ii) at risk of Fabry disease based on one or more mutations in galactosidase alpha (GLA) gene, including but not limited to g.1181T>G, g.1271C>T, g.1316A>G, g.5156A>G, g.5165T>C, g.5171T>C, g.5173G>A, g.5180G>A, g.5189C>T, g.5198G>A, g.5236T>C, g.7300A>C, g.7311G>A. g.7326G>A, g.7343T>G, g.7387G>T, g.7408A>T, g.8378G>A, g.10137T>G, g.10279A>G, g.10568G>T, g.10601A>G, g.11134C>T. g.11174G>A, g.8414T>C, 5204delC, 8386del9, 10268delA, 11035delAT, 11053delGA, 11055delT, R404del, 11072insC, g.1312TGCAC>GCTCG, and/or g. 5115GGCAGAGCTCATG>GCAGAGCCA (see web site fabrygenphen.com);
(jj) at risk of Gaucher disease caused by mutations in beta-glucocerebrosidase (GBA) gene, including but not limited to c.72delC, c.84insGG, c.254G>A, c.371T>G, c.754T>A, c.764T>A, c.827C>T, c.957G>C, c.1195G>C, c.1342G>C, c.1448T>C, c.1504C>T, c.1603T>C, c.1604G>A, c.1459G>A, c.1504C>T, c.3170A>C, c.3119G>A, c.3548T>A, c.3931G>A, c.4113T>A, c.5309G>A, c.5912G>T, c.5958A>T, p.V15L, p.G46E, p.N188S, p.P122S, p.K157Q, p.A309V, p.N370S, p.L371V, p.G377S, p.L444P, p.R119Q, p.R120Q, p.V394L, p.D409H, p.R463C, p.L444P, p.L483P, p.R535C, and/or IVS10-1G-A (see CCHMC Molecular Genetics Laboratory Mutation Database web site research.cchmc.org/LOVD2/home.php?select_db=GBA);
(kk) at risk of Niemann-Pick disease Types A and B based on one or more mutations in acid sphingomyelinase (SMPD1) gene;
(ll) at risk of Niemann-Pick disease Type C based on one or more mutations in NPC intracellular cholesterol transporter 1 (NPC1) gene, including but not limited to c.3503G>A, c.3485G>C, c.3467A>G, c.3182T>C, c.3160G>A, c.3104C>T, c.3056A>G, c.3019C>G, c.2974G>T, c.2819C>T, and/or c.2324A>C;
(mm) at risk of Niemann-Pick disease Type C based on one or more mutations in NPC intracellular cholesterol trasporter 2 (NPC2) gene;
(nn) at risk of GM1 gangliosidosis based on one or more mutations in beta-galactosidase 1 (GLB1) gene;
(oo) at risk of GM2 gangliosidosis (including but not limited to Sandhoff and Tay-Sachs) based on one or more mutations in beta-hexosamidase A (HEXA) gene, including but not limited to c.1278insTATC, c.1496G>A, c.1073+1G>A, c.1422G>C, c.533G>A, c.1510delC, c.805G>A, c.1514G>A, IVS11+5G>A, c.410G>A, c.796T>G, c.1057G>C;
(pp) at risk of GM2 gangliosidosis (including but not limited to Sandhoff and Tay-Sachs) based on one or more mutations in GM2 ganglioside activator (GM2A) gene;
(qq) at risk of mucopolysachariddoses (MPS) type I (Hurler disease) based on one or more mutations in alpha-L iduronidase (IDUA) gene, including but not limited to p.Q70X, and/or p.W402X;
(rr) at risk of mucopolysachariddoses (MPS) type II (Hunter disease) based on one or more mutations in iduronate 2-sulfatase (IDS) gene;
(ss) at risk of mucopolysachariddoses (MPS) type IIIa (Sanfilippo A) based on one or more mutations in N-sulfoglucosamine sulfohydrolase (SGSH) gene;
(tt) at risk of mucopolysachariddoses (MPS) type IIIB (Sanfilippo B) based on one or more mutations in alpha-N-acetylglucosaminidase (NAGLU) gene;
(uu) at risk of mucopolysachariddoses (MPS) type IIIc (Sanfilippo C) based on one or more mutations in heparan-alpha-glucosaminide N-acetyltransferase (HGSNAT) gene;
(vv) at risk of mucopolysachariddoses (MPS) type IIId (Sanfilippo D) based on one or more mutations in N-acetylglucosamine-6-sulfatase (GNS) gene;
(ww) at risk of mucopolysachariddoses (MPS) type IVA (Morquio A) based on one or more mutations in N-acetylgalactosamine-6-sulfatase (GALNS) gene,
(xx) at risk of mucopolysachariddoses (MPS) type IVB based on one or more mutations in beta-galactosidase (GLB1) gene;
(yy) at risk of mucopolysachariddoses (MPS) type VI based on one or more mutations in arylsulfatase B (ARSB) gene;
(zz) at risk of mucopolysachariddoses (MPS) type VII based on one or more mutations in beta-glucoronidase (GUSB) gene;
(aaa) at risk of mucopolysachariddoses (MPS) type IX based on one or more mutations in hyaluronidase 1 (HYAL1) gene;
(bbb) at risk of mucolipidosis III (I-cell) based on one or more mutations in N-acetylglucosamine-1-phosphate transferase subunits alpha and beta (GNPTAB) gene;
(ccc) at risk of mucolipisosis IV based on one or more mutations in mucolipin 1 (MCOLN1) gene;
(ddd) at risk of multiple sulfatase deficiency based on one or more mutations in sulfatase modifying factor 1 (SUMF1) gene;
(eee) at risk of sialidosis based on one or more mutations in neuraminidase 1 (NEU1); galactosialidosis caused by mutations in cathepsin A (CTSA) gene;
(fff) at risk of β-mannosidosis based on one or more mutations in alpha-mannosidase (MAN2B1) gene;
(ggg) at risk of β-mannosidosis based on one or more mutations in beta-mannosidase MANBA gene;
(hhh) at risk of apartylglucosaminuria based on one or more mutations in aspartylglucosaminidase (AGA) gene;
(iii) at risk of fucosidosis based on one or more mutations in alpha-L-fucosidase (FUCA1) gene;
(jjj) at risk of Schindler disease based on one or more mutations in alpha-N-acetylgalactosaminidase (NAGA) gene;
(kkk) at risk of metachromatic leukodystrophy based on one or more mutations in arylsulfatase A (ARSA) gene, including but not limited to c.459+1G>A, p.P426L, p.A212V, p.R244C, p.R390W, p.P426L, p.S95N, p.G119R, p.D152Y, p.R244H, p.S250Y, p.A314T, p.R384C, p.R496H, p.K367N;
(lll) at risk of metachromatic leukodystrophy based on one or more mutations in prosaposin (PSAP) gene;
(mmm) at risk of globoid cell leukodystrophy (Krabbe disease) based on one or more mutations in galactosylceramidase (GALC) gene, including but not limited to c.550C>T, c.334A>G, c.1162-4del, c.330C>T, c.61G>C, c.913A>G, c.984G>A, c.956A>G, c.1350C>T, c.1671-15C>T, and/or c.1685T>C;
(nnn) at risk of Farber lipogranulomatosis based on one or more mutations in acid ceramidase (ASAH1) gene;
(ooo) at risk of Wolman and/or cholesteryl ester storage disease based on one or more mutations in lipase A (LAL) gene;
(ppp) at risk of pycnodystostosis based on one or more mutations in cathepsin K (CTSK) gene;
(qqq) at risk of cystinosis based on one or more mutations in cystinosin (CTNS) gene;
(rrr) at nrisk of Salla disease based on one or more mutations in solute carrier family 17 member 5 (SLC17A5) gene;
(sss) at risk of Danon disease based on one or more mutations in lysosomal associated membrane protein-2 (LAMP2) gene;
(ttt) at risk of Griscelli disease Type 1 based on one or more mutations in myosin VA (MYO5A) gene;
(uuu) at risk of Griscelli disease Type 2 based on one or more mutations in RAB27a member RAS oncogene family (RAB27A) gene;
(vvv) at risk of Griscelli disease Type3 based on one or more mutations in melanophilin (MLPH) gene;
(www) at risk of Hermansky Pudliak Disease based on one or more mutations in a gene selected from the group consisting of biogenesis of lysosomal organelles complex 3 subunit 1 (HPS1), AP-2 complex subunit beta-1 (AP3B1), biogenesis of lysosomal organelles complex 2 subunit 1 (HPS3), biogenesis of lysosomal organelles complex 3 subunit 2 (HPS4), biogenesis of lysosomal organelles complex 2 subunit 2 (HPS5), biogenesis of lysosomal organelles complex 2 subunit 3 (HPS6), dystrobrevin bindin protein 1 (DTNBP1), biogenesis of lysosomal organelles complex 1 subunit 3 (BLOC1S3), biogenesis of lysosomal organelles complex 1 subunit 6 (BLOCIS6, PLDN), and/or adaptor related protein complex 3 subunit delta 1 (AP3D1); and/or
(xxx) at risk of Chédiak-Higashi syndrome based on one or more mutations in lysosomal trafficking regulator (LYST) gene.

In one embodiment, the method comprises administering an amount effective of a SORT1 inhibitor to treat or limit development of the lysosomal storage disorder. As disclosed herein, the inventors have demonstrated that sortilin-inhibition leads to decreased accumulation of lysosomal storage substrates, and thus can be used to treat or limit development of the lysosomal storage disorders.

In one embodiment, the SORT1 inhibitor comprises a compound of the formula (I):

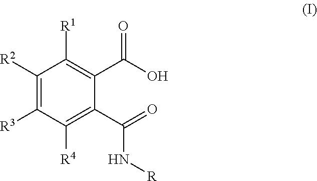

or pharmaceutically acceptable salts thereof, wherein
R¹ is hydrogen, halogen, C₁-C₆ alkyl, or C₁-C₆ haloalkyl;
R² is hydrogen, halogen, C₁-C₆ alkyl, C₁-C₆ haloalkyl, —NO₂, —CN, —OH, —SH, —NH₂, —NH(C₁-C₆ alkyl), —N(C₁-C₆ alkyl)₂, C₁-C₆ alkoxy, C₁-C₆ haloalkoxy, aryl optionally substituted with one or more R₅, or heteroaryl optionally substituted with one or more R₅;
R³ is hydrogen, halogen, C₁-C₆ alkyl, C₁-C₆ haloalkyl, —NO₂, —CN, —OH, —SH, —NH₂, —NH(C₁-C₆ alkyl), —N(C₁-C₆ alkyl)₂, C₁-C₆ alkoxy, or C₁-C₆ haloalkoxy;
R⁴ is hydrogen, halogen, C₁-C₆ alkyl, or C₁-C₆ haloalkyl; and
R is C₁-C₆ alkyl, aryl optionally substituted with one or more R₅, or heteroaryl optionally substituted with one or more R₅,
wherein
   each R₅ is independently selected from the group consisting of halogen, —NO₂, —CN, C₁-C₆ alkyl, C₁-C₆ haloalkyl, —OH, C₁-C₆ alkoxy, and C₁-C₆ haloalkoxy.

In various embodiments, R¹ is hydrogen or C₁-C₃ alkyl; R¹ is hydrogen or methyl; or R¹ is hydrogen.

In other embodiments, R² is hydrogen, halogen, C₁-C₃ alkyl, C₁-C₃ haloalkyl, —NO₂, —CN, —OH, —SH, —NH₂, —NH(C₁-C₃ alkyl), —N(C₁-C₃ alkyl)₂, C₁-C₃ alkoxy, C₁-C₃ haloalkoxy, or phenyl optionally substituted with one or more R₅; R² is hydrogen, halogen, C₁-C₃ alkyl, C₁-C₃ haloalkyl, —NO₂, —OH, —NH₂, —NH(C₁-C₃ alkyl), —N(C₁-C₃ alkyl)₂, C₁-C₃ alkoxy, or C₁-C₃ haloalkoxy; R² is hydrogen, halogen, C₁-C₃ alkyl. C₁-C₃ haloalkyl, —NO₂, C₁-C₃ haloalkoxy or phenyl; R² is hydrogen, halogen, C₁-C₃ alkyl, or C₁-C₃ haloalkyl; R² is halogen, C₁-C₃ alkyl, or C₁-C₃ haloalkyl; R² is halogen or C₁-C₃ haloalkyl; R² is bromo, chloro, or —CF₃; or R² is —CF₃.

In some embodiment, R³ is hydrogen, halogen, C₁-C₃ alkyl, or C₁-C₃ haloalkyl; R³ is hydrogen, bromo, chloro, methyl, or —CF₃; or R³ is hydrogen.

In other embodiments, R⁴ is hydrogen or C₁-C₃ alkyl; or R⁴ is hydrogen.

In one embodiment, R¹ is hydrogen, R² is halogen, C₁-C₃ alkyl, or C₁-C₃ haloalkyl, R³ is hydrogen, and R⁴ is hydrogen.

In various embodiments, R is phenyl or 6-membered heteroaryl, each optionally substituted with one or more R₅; R is phenyl, pyridinyl, or pyrimidinyl, each optionally substituted with one or more R₅; R is phenyl, pyridinyl, or pyrimidinyl, each optionally substituted with one or two R₅; or R is

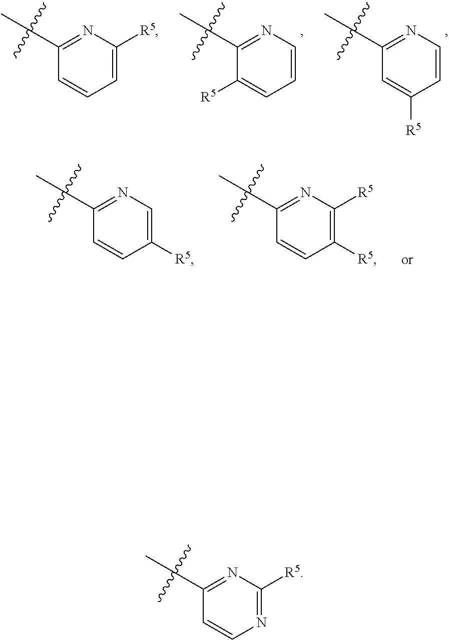

In other embodiments, R₅ is halogen, C₁-C₆ alkyl, C₁-C₆ haloalkyl, C₁-C₆ alkoxy, and C₁-C₆ haloalkoxy; wherein R₅ is halogen, C₁-C₃ alkyl, C₁-C₃ haloalkyl, C₁-C₃ alkoxy, and C₁-C₃ haloalkoxy; R₅ is bromo, chloro, methyl, —CF₃, or methoxy; or R₅ is chloro, methyl, or methoxy.

In one embodiment, R is

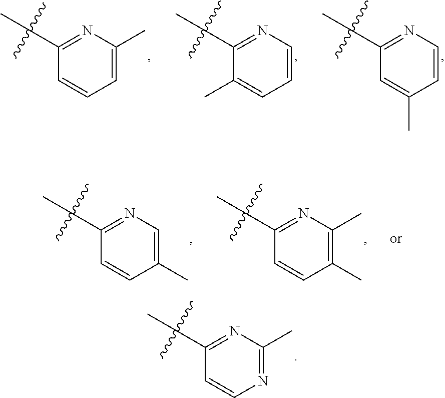

In another embodiment, R is

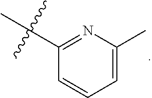 [SMILES: *c1cccc(C)n1]

In various specific embodiments, the compound of formula (I) is selected from the group consisting of:

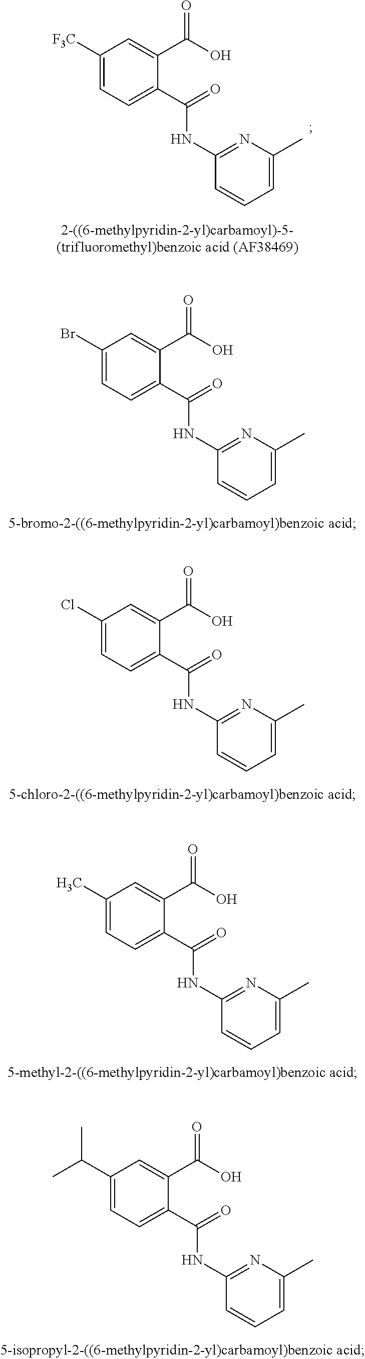

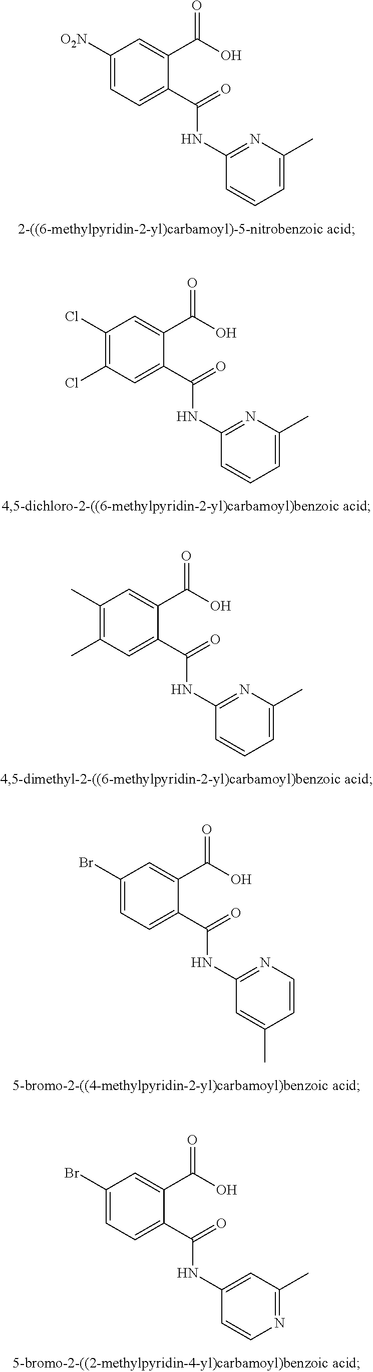

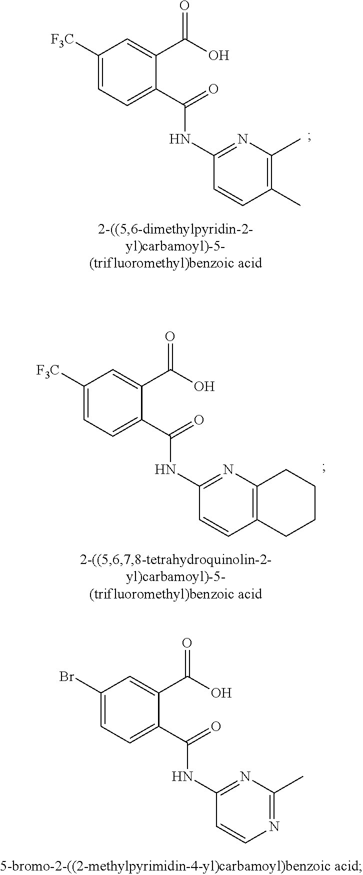 [SMILES: Cc1ccc(NC(=O)c2ccc(C(F)(F)F)cc2C(=O)O)nc1C ; O=C(O)c1cc(C(F)(F)F)ccc1C(=O)Nc1ccc2c(n1)CCCC2 ; Cc1nccc(NC(=O)c2ccc(Br)cc2C(=O)O)n1]

or pharmaceutically acceptable salts thereof.

In one specific embodiment, the SORT1 inhibitor is:

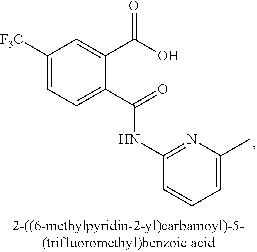 [SMILES: Cc1cccc(NC(=O)c2ccc(C(F)(F)F)cc2C(=O)O)n1]

or pharmaceutically acceptable salts thereof. This compound is also referred to herein as AF38469 (CAS RN: 1531634-31-7; PubChem CID: 72706115) and described by Schroder, T. J. et al. (Bioorg Med Chem Lett. 2014, 24 (1), 177-180). AF38469 is commercially available from, for example, VulcanChem (Altadena, CA).

In another embodiment, the SORT1 inhibitor comprises an inhibitor selected from the group consisting of AF38469 or N-substituted-5-substituted pthalamic acids, AF40431 (N-[(7-hydroxy-4-methyl-2-oxo-2H-chromen-8-yl)methyl]-L-leucine) or substituted versions thereof; (S)-2-(3,5-dichlorobenzamido)-5,5-dimethylhexanoic acid or derivatives such as (S)-2-(4-chloro-1H-pyrrole-2-carboxamido)-5,5-dimethylhexanoic acid and (S)-5-5-dimethyl-2-(6-phenoxynicotinamido)hexanoic acid or substituted versions thereof; 1-benzyl-3-(tert-butyl)-1H-pyrazole-5-carboxylic acid or substituted versions thereof, SORT1 small interfering RNAs, small internally segmented interfering RNAs, short hairpin RNAs, microRNAs, and/or antisense oligonucleotides; cas9 repressors or other cas (CRISPR) repressors targeting the SORT1 locus; anti-SORT1 antibodies or antibody fragments thereof; combinations thereof; or pharmaceutically acceptable salts thereof; in particular AF38469 or a pharmaceutically acceptable salt thereof.

For nucleic acid constructs encoding inhibitory nucleic acids or cas9 repressors or other cas (CRISPR) repressors targeting the SORT1 locus, the nucleic acid sequences, such as DNA sequences, may be expressed by any suitable expression vector, including but not limited to viral vectors including adeno-associated viruses (AAVs, e.g. AAV1, AAV2, AAV9).

Exemplary small molecule SORT1 inhibitors are commercially available and methods for their synthesis are known in the art (Schroder et al. 2014; Stachel et al. 2020). AF40431 is commercially available from, for example, MedChemExpress (Monmouth Junction, NJ). Synthesis of exemplary N-substituted-5-substituted pthalamic acids SORT1 inhibitors is disclosed in, for example, Stachel, et al. 2020.

In another embodiment, the method comprises administering an amount effective of a NGRF modulator to treat or limit development of the lysosomal storage disorder. As disclosed herein, the inventors show that NGRF modulators lead to decreased accumulation of lysosomal storage substrates, and thus can be used to treat or limit development of the lysosomal storage disorders.

In various embodiments, the NGFF modulator comprises a compound of the formula (II):

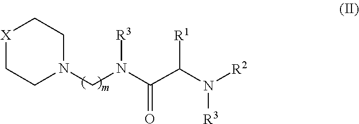

or pharmaceutically acceptable salts thereof, wherein
m is an integer in a range of 1 to 8;
X is O or NR, where R is hydrogen or C₁-C₆ alkyl;
R¹ is hydrogen, halogen, C₁-C₈ alkyl optionally substituted with one or more R⁴, C₁-C₆ haloalkyl, —OH, —NH₂, —NH(C₁-C₆ alkyl), —N(C₁-C₆ alkyl)₂, C₁-C₆ alkoxy, C₁-C₆ haloalkoxy, cycloalkyl(C₀-C₆ alkyl)- optionally substituted with one or more R₅, or aryl(C₀-C₆ alkyl)- optionally substituted with one or more R₅;
R² is hydrogen, C₁-C₆ alkyl optionally substituted with one or more R⁴, cycloalkyl(C₀-C₆ alkyl)- optionally substituted with one or more R₅, aryl(C₀-C₆ alkyl)- optionally substituted with one or more R₅, or heteroaryl(C₀-C₆ alkyl)- optionally substituted with one or more R₅; and
each R³ is independently hydrogen or C₁-C₆ alkyl;
wherein
   each R₄ is independently selected from the group consisting of —NO₂, —CN, —OH, C₁-C₆ alkoxy, and C₁-C₆ haloalkoxy; and
   each R₅ is independently selected from the group consisting of halogen, —NO₂, —CN, C₁-C₆ alkyl, C₁-C₆ haloalkyl, —OH, C₁-C₆ alkoxy, and C₁-C₆ haloalkoxy.

In various embodiments, X is NR, and R is hydrogen or methyl; or X is O. In other embodiments, m is 1, 2, 3, or 4; or m is 1, 2, or 3; or m is 2. In other embodiments, the compound of formula (II) is of formula:

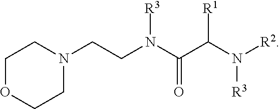

In various embodiments, each R³ is independently hydrogen or methyl; or each R³ is independently hydrogen.

In another embodiment, the compound of formula (II) is of formula:

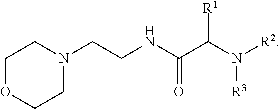

In one embodiment, R³ is hydrogen or methyl. For example, R³ is hydrogen. In another example, R³ is methyl.

In another embodiment, the compound of formula (II) is of formula:

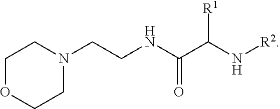

In one embodiment, R² is hydrogen or C₁-C₆ alkyl optionally substituted with one or more R⁴. In another embodiment, R² is hydrogen or C₁-C₃ alkyl. In a further embodiment, R² is hydrogen or methyl, or R² is hydrogen.

In other embodiments, R¹ is hydrogen, C₁-C₈ alkyl optionally substituted with one or more R⁴, C₁-C₆ haloalkyl, cycloalkyl(C₀-C₆ alkyl)- optionally substituted with one or more R₅, or aryl(C₀-C₆ alkyl)- optionally substituted with one or more R₅. In another embodiment, R¹ is C₁-C₈ alkyl optionally substituted with one or more R⁴, C₁-C₆ haloalkyl, cycloalkyl(C₀-C₆ alkyl)- optionally substituted with one or more R₅, or aryl(C₀-C₆ alkyl)- optionally substituted with one or more R₅. In a further embodiment, R¹ is C₁-C₈ alkyl optionally substituted with one or more R₄ (such as C₁-C₆ alkyl optionally substituted with one or more R₄). In one embodiment, R¹ is C₁-C₆ alkyl.

In various specific embodiments, the compound of formula (II) is selected from the group consisting of

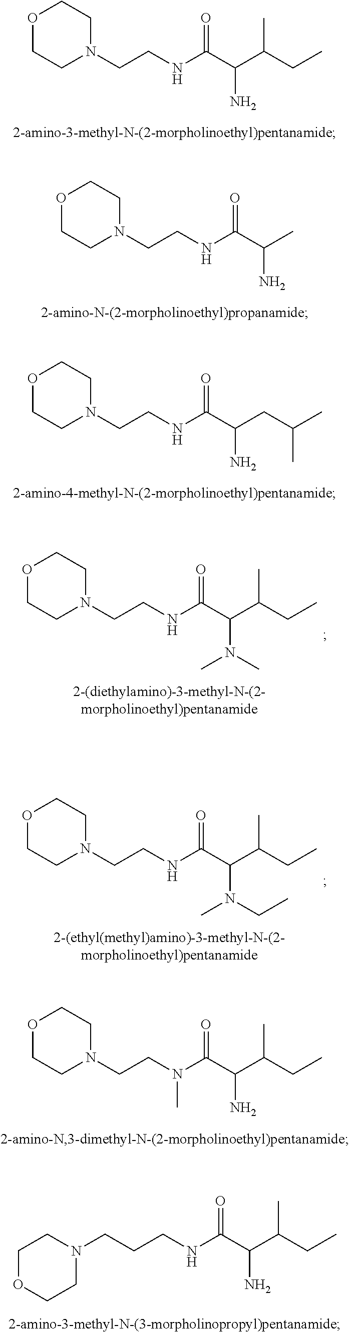

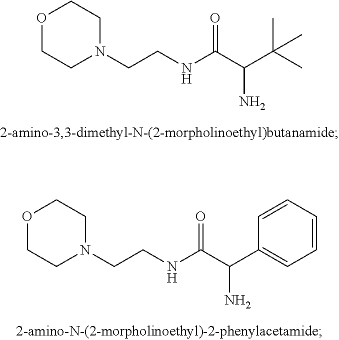 [SMILES: CC(C)(C)C(N)C(=O)NCCN1CCOCC1 ; NC(C(=O)NCCN1CCOCC1)c1ccccc1]

or pharmaceutically acceptable salts thereof.

In one embodiment, the NGFF modulator is:

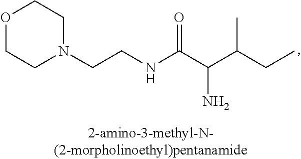 [SMILES: CCC(C)C(N)C(=O)NCCN1CCOCC1]

or pharmaceutically acceptable salts thereof.

In one embodiment, the NGFF modulator is:

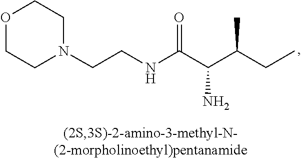 [SMILES: CC[C@H](C)[C@H](N)C(=O)NCCN1CCOCC1]

or pharmaceutically acceptable salts thereof. This compound is also known as LM11A-31 or N-[2-(morpholin-4-yl)ethyl]-L-isoleucinamide (CAS RN: 1243259-19-9; PubChem CID: 18604758). LM11A-31 is commercially available from, for example, Cayman Chemical (Ann Arbor, MI). The compound “LM11A-31” as used herein may be enantiomerically pure (e.g., based on L-isoleucine), enantiomerically enriched, a mixture of two or more enantiomers, or racemic. In certain embodiments, LM11A-31 is the enantiomer based on L-isoleucine. In certain embodiments, LM1TA-31 is the enantiomer based on D-isoleucine. In certain embodiments, LM11A-31 is racemic. In addition, “LM11A-31” as used herein may be provided as a free base or as a salt (such as a hydrochloride, sulfate, etc.).

In another embodiment, the NGRF modulator is LM11A-24 (CAS RN: 106522-85-4; PubChem CID: 3653705) having the following structure

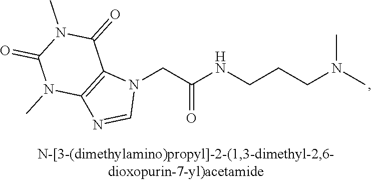 [SMILES: CN(C)CCCNC(=O)Cn1cnc2c1c(=O)n(C)c(=O)n2C]

or pharmaceutically acceptable salts thereof. LM11A-24 is commercially available from, for example, VulcanChem (Altadena, CA).

In another embodiment, the NGRF modulator is selected from the group consisting of LM11A-31 and substituted versions thereof; LM11A-24 and substituted versions thereof; NGFR-binding peptides or peptide modulators of NGFR signaling, or nucleic acid constructs encoding such peptide modulators, including but not limited to peptides derived from NGFR ligands such as the pro forms of brain-derived neurotrophic factor (BDNF) and nerve growth factor (NGF); anti-NGFR antibodies or antibody fragments; combinations thereof; or pharmaceutically acceptable salts thereof; particularly LM11A-31 or pharmaceutically acceptable salts thereof.

Exemplary small molecule NGFR ligands are commercially available and methods for their synthesis are known in the art (Yang, et al., PLoS One, 2008; 3(11):e3604. doi: 10.1371/journal.pone.0003604. Epub 2008 Nov. 3).

In another embodiment, the method comprises administering an amount effective of a GLP-1R agonist to treat or limit development of the lysosomal storage disorder. As disclosed herein, the inventors show that GLP-1R agonists lead to decreased accumulation of lysosomal storage substrates, and thus can be used to treat or limit development of the lysosomal storage disorders.

In one embodiment, the GLP-TR agonist is selected from the group consisting of semaglutide, exenatide, liraglutide, lixisenatide, albiglutide, dulaglutide, taspoglutide, compounds that inhibit dipeptidyl peptidase-4 (DDP-4, which degrades GLP-1) including but not limited to sitagliptin, vildagliptin, saxagliptin, linagliptin, gemigliptin, anagliptin, teneligliptin, alogliptin, trelagliptin, omarigliptin, evogliptin, gosogliptin, dutogliptin, and berberine; compounds that elicit GLP-1 secretion including metformin; GLP-1R agonist peptides, or nucleic acid constructs encoding such agonist peptides, including but not limited to peptides consisting of at least eight contiguous amino acids of GLP-1R ligands such as glucagon-like peptide-1 (GLP-1, human amino acid sequence: HDEFERHAEGTFTSDVSSYLEGQAAKEFIAWLVKGRG; SEQ ID NO:1) and exendin-4 (amino acid sequence: HGEGTFTSDLSKQMEEEAVRLFIEWLKNGGPSSGAPPPS (SEQ ID NO:2)), or mutated versions thereof; anti-GLP-TR agonist antibodies or antibody fragments; combinations thereof; or pharmaceutically acceptable salts thereof.

In one embodiment, the GLP-TR agonist is selected from the group consisting of semaglutide, exenatide, liraglutide, lixisenatide, albiglutide, dulaglutide, and taspoglutide, or a pharmaceutically acceptable salt thereof. In a specific embodiment, the GLP-TR agonist is semaglutide or a pharmaceutically acceptable salt thereof.

Semaglutide is a glucagon like peptide 1 (GLP-1) receptor peptidomimetic agonist. Structurally it is a modified analogue of glucagon-like peptide 1-(7-37) with amino acids at positions 8 and 34 replaced by α-aminobutyric acid and arginine respectively, and Lys26 is acylated with stearic diacid. The main protraction mechanism of semaglutide is albumin binding, facilitated by modification of position 26 lysine with a hydrophilic spacer and a C18 fatty di-acid. Furthermore, semaglutide is modified in position 8 to provide stabilization against degradation by the enzyme dipeptidyl-peptidase 4 (DPP-4). A minor modification was made in position 34 to ensure the attachment of only one fatty di-acid.

### GLP-1 AA Sequence

(SEQ ID NO: 3)
His-Ala-Glu-Gly-Thr-Phe-Thr-Ser-Asp-Val-
Ser-Ser-Tyr-Leu-Glu-Gly-Gln-Ala-Ala-Lys-
Glu-Phe-Ile-Ala-Trp-Leu-Val-Lys-Gly-Arg.

The compounds may be administered as the sole active therapeutic agent, or may be administered in combination with one or more other active therapeutic agents. In one embodiment, the method comprises administering to the subject an amount effective of 2 or all 3 of a sortilin (SORT1) inhibitor, a p75 neurotrophin receptor (NGFR) modulator, and/or a glucagon-like peptide-1 receptor (GLP-1R) agonist as recited in any embodiment herein.

In one such embodiment, the method comprises administering to the subject an amount effective of AF38469 or a pharmaceutically acceptable salt thereof, and LM11A-31 or a pharmaceutically acceptable salt thereof, to treat or limit development of the lysosomal storage disorder.

In another embodiment, the method comprises administering to the subject an amount effective of AF38469 or a pharmaceutically acceptable salt thereof, semaglutide or a pharmaceutically acceptable salt thereof, to treat or limit development of the lysosomal storage disorder.

In a further embodiment, the method comprises administering to the subject an amount effective of AF38469 or a pharmaceutically acceptable salt thereof, LM11A-31 or a pharmaceutically acceptable salt thereof, and semaglutide or a pharmaceutically acceptable salt thereof, to treat or limit development of the lysosomal storage disorder.

In one embodiment, the method comprises administering to the subject an amount effective of LM11A-31 or a pharmaceutically acceptable salt thereof, and semaglutide or a pharmaceutically acceptable salt thereof, to treat or limit development of the lysosomal storage disorder.

In another embodiment, the methods may further comprise administration/inclusion administering one or more gene therapy products that encode proteins implicated in lysosomal storage disorders. In one embodiment, the gene therapy product encodes PPT1, TPP1, CLN3, CLN4 (DNAJC5), CLN5, CLN6, CLN7 (MFSD8), CLN8, CLN10 (CTSD), CLN11, CLN12 (ATP13A2), CLN13 (CTSF), CLN14 (KCTD7) or functional fragment thereof In another embodiment, the gene therapy product encodes lysosomal alpha-glucosidase (GAA), alpha-galactosidase A (GLA), glucosylceramidase beta (GBA), acid sphingomyelinase (SMPD1), NPC intracellular cholesterol transporter 1 (NPC1), NPC intracellular cholesterol transporter 2 (NPC2), beta-galactosidase 1 (GLB1), beta-hexosamidase A (HEXA), GM2 ganglioside activator (GM2A), alpha-L iduronidase (IDUA), iduronate 2-sulfatase (IDS), N-sulfoglucosamine sulfohydrolase (SGSH), N-acetylglucosaminidase (NAGLU), heparan-alpha-glucosaminide N-acetyltransferase (HGSNAT), N-acetylglucosamine-6-sulfatase (GNS), N-acetylgalactosamine-6-sulfatase (GALNS), arylsulfatase B (ARSB), beta-glucoronidase (GUSB), hyaluronidase 1 (HYAL1), N-acetylglucosamine-1-phosphate transferase subunits alpha and beta (GNPTAB), mucolipin 1 (MCOLN1), sulfatase modifying factor 1 (SUMF1), neuraminidase 1 (NEU1), cathepsin A (CTSA), alpha-mannosidase (MAN2B1), beta-mannosidase (MANBA), aspartylglucosaminidase (AGA), alpha-L-fusocidase (FUCA1), alpha-N-acetylgalactosaminidase (NAGA), arylsulfatase A (ARSA), prosaposin (PSAP), galactosylceramidase (GALC), acid ceramidase 1 (ASAH1), lipase A (LAL), cathepsin K (CTSK), cystinosin (CTNS), solute carrier family 17 member 5 (SLC17A5), lysosomal associated membrane protein-2 (LAMP2), myosin VA (MYO5A), Rab27a member RAS oncogene family (RAB27A), melanophilin (MLPH), biogenesis of lysosomal organelles complex 3 subunit 1 (HPS1), AP-2 complex subunit beta-1 (AP3B1), biogenesis of lysosomal organelles complex 2 subunit 1 (HPS3), biogenesis of lysosomal organelles complex 3 subunit 2 (HPS4), biogenesis of lysosomal organelles complex 2 subunit 2 (HPS5), biogenesis of lysosomal organelles complex 2 subunit 3 (HPS6), dystrobrevin binding protein 1 (DTNBP1), biogenesis of lysosomal organelles complex 1 subunit 3 (BLOCIS3, PLDN) adaptor related protein complex 3 subunit delta 1 (AP3D1), lysosomal trafficking regulator (LYST), or functional fragments thereof.

The therapeutic agents can be formulated as separate compositions that are given at the same time or different times, or the therapeutic agents can be given as a single composition. The therapeutic agents can be used in combination with one or more other compounds useful for carrying out the methods of the disclosure.

As used herein, an “amount effective” refers to an amount of the composition that is effective for treating the relevant disorder. The amount of a compound which constitutes a “therapeutically effective amount” will vary depending on the compound, the disorder and its severity, and the age of the subject to be treated, but can be determined routinely by one of ordinary skill in the art.

As used herein, limiting development of a disorder means to prevent or to minimize development of the disorder in a subject at risk of developing the disorder. Subjects at risk of developing the disorder include those disclosed above.

As used herein, “treat” or “treating” means accomplishing one or more of the following in an individual that already has a lysosomal storage disorder: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); (e) limiting or preventing recurrence of symptoms in patients that were previously symptomatic for the disorder(s); and (f) prolonging survival.

Symptoms of lysosomal disorders to be treated may include developmental delay; dementia; psychosis; memory impairment; visual impairment; motor disturbances including ataxia, abnormal gait and/or frequent falls, and loss of motor function; speech impairment; seizures; tremors; sleep disturbance; incontinence; hyperactivity; irritability; dysphagia; manifestations of cardiomyopathy, cardiomegaly, and cardiovascular disease including shortness of breath, fatigue, stroke, and impaired performance on physically-demanding tasks such as walking; symptoms of peripheral neuropathy such as neuropathic pain, numbness, and tingling; symptoms of impaired kidney function including fatigue, shortness of breath, confusion, nausea, and irregular heartbeat; anemia; liver and/or spleen enlargement; symptoms of impaired gastrointestinal function; and symptoms of impaired pulmonary function including wheezing and pneumonia.

In some embodiments, the methods may results in stabilization, reduced progression, or improvement in one of more biomarkers of disease progression or status. Specifically, in comparison to the subject before treatment or in comparison to an untreated subject, the methods provided herein may result in one or more of the following: reduced or stabilized biofluid levels of lysosomal storage material or substrates (e.g. globotriaosylceramide in Fabry disease), reduced or stabilized biofluid levels of LSD-specific or disease-specific biomarkers (e.g. chitotriosidase in Gaucher disease), reduced or stabilized biofluid levels of lysosomal enzymes or lysosomal glycoproteins (e.g. cathepsin D, cathepsin S, progranulin, tripeptidyl peptidase 1), reduced or stabilized biofluid levels of markers of skeletal muscle damage (e.g. troponin T), or reduced or stabilized biofluid levels of markers of cardiac muscle damage (e.g. troponin I), reduced or stabilized cerebrospinal fluid or blood levels of neurofilament light (NFL), reduced or stabilized cerebrospinal fluid or blood levels of ubiquitin c-terminal hydrolase L1 (UCHL1), reduced or stabilized cerebrospinal fluid or blood levels of gamma-enolase (ENO2), reduced or stabilized cerebrospinal fluid or blood levels of Tau including phosphorylated Tau, reduced or stabilized brain ventricle enlargement as measured by neuroimaging, reduced grey matter hypointensities or hyperintensities measured by MRI, reduced white matter hypointensities or hyperintensities measured by MRI, reduced periventricular hyperintensity measured by MRI, reduced or stabilized cerebellar atrophy as measured by neuroimaging, reduced or stabilized cortical atrophy as measured by neuroimaging, reduced or stabilized reductions in whole brain volume as measured by neuroimaging, reduced or stabilized corpus callosum thinning as measured by neuroimaging, reduced or stabilized deterioration in white matter integrity as reflected by changes in fractional anisotropy, radial diffusivity, and axial diffusivity using diffusion tensor imaging (DTI), restoration of brain metabolite and biomolecule levels as measured by magnetic resonance spectroscopy (MRS), reduced or stabilized [¹⁸F]FEPPA uptake observed by positron emission tomography, improved or stabilized brain glucose uptake observed by positron emission tomography, reduced or stabilized deterioration in retinal function as measured by electroretinogram (ERG) or visual evoked potential (VEP), or reduced or stabilized retinal degeneration as measured by optical coherence tomography (OCT).

In embodiments where the subject suffers from an NCL/Batten Disease, the methods may result in stabilization, reduced progression, or improvement in one or more of the scales that are used to evaluate progression and/or improvement in Batten disease, including but not limited to motor function, language function, cognitive function, and survival e.g. the Hamburg Motor and Language Scale (as described in Marshall et al., Neurology. 2005 65(2):275-279) or the Unified Batten Disease Rating System (UBDRS) [including the UBDRS physical assessment scale, the UBDRS seizure assessment scale, the UBDRS behavioral assessment scale, the UBDRS capability assessment scale, the UBDRS sequence of symptom onset, and the UBDRS Clinical Global Impressions (CGI)]; or the Pediatric Quality of Life Scale (PEDSQOL) scale. In comparison to the subject before treatment or in comparison to an untreated subject, the methods provided herein may result in one or more of the following: reduced or slowed degeneration of photoreceptors; reduced or slowed retinal degeneration; increased number of retinal photoreceptors compared to an untreated subject; reduced or slowed cellular accumulation of storage material; reduced or slowed cellular accumulation of mitochondrial ATP Synthase Subunit C, saposin A, or saposin D; reduced or slowed glial activation (astrocytes and/or microglia) activation; reduced or slowed astrocytosis; reduced or stabilized cerebrospinal fluid or blood levels of neurofilament light (NFL); reduced or stabilized cerebrospinal fluid or blood levels of ubiquitin c-terminal hydrolase L1 (UCHL1); reduced or stabilized cerebrospinal fluid or blood levels of gamma-enolase (ENO2); reduced or stabilized biofluid levels of lysosomal storage material or substrates; reduced or stabilized biofluid levels of lysosomal enzymes or lysosomal glycoproteins (e.g. cathepsin D, cathepsin S, progranulin, tripeptidyl peptidase 1); reduced or stabilized biofluid levels of markers of cardiac muscle damage (e.g. troponin I); increased or stabilized white blood cell lysosomal enzyme (e.g. palmitoyl-protein thioesterase 1 [PPT1], tripeptidyl-peptidase 1 [TPP1], cathepsin D [CTSD]) activity levels; reduced or stabilized symptoms of dementia, psychosis, memory impairment, visual impairment, motor disturbances, speech impairment, seizures, tremors, sleep disturbance, incontinence, hyperactivity, irritability, and/or dysphagia; reduced or stabilized brain ventricle enlargement as measured by neuroimaging; reduced grey matter hypointensities or hyperintensities measured by MRI; reduced white matter hypointensities or hyperintensities measured by MRI; reduced periventricular hyperintensity measured by MRI; reduced or stabilized cerebellar atrophy as measured by neuroimaging; reduced or stabilized cortical atrophy as measured by neuroimaging; reduced or stabilized reductions in whole brain volume as measured by neuroimaging; reduced or stabilized corpus callosum thinning as measured by neuroimaging; reduced or stabilized deterioration in white matter integrity as reflected by changes in fractional anisotropy, radial diffusivity, and axial diffusivity using diffusion tensor imaging (DTI); or restoration of brain metabolite and biomolecule levels as measured by magnetic resonance spectroscopy (MRS); reduced or stabilized [¹⁸F]FEPPA uptake observed by positron emission tomography; improved or stabilized brain glucose uptake observed by positron emission tomography; reduced or stabilized deterioration in retinal function as measured by electroretinogram (ERG) or visual evoked potential (VEP); reduced or stabilized retinal degeneration as measured by optical coherence tomography (OCT); and/or improvement or stabilization in 36-Item Short Form Survey (SF-36) score.

In embodiments where the subject suffers from Gaucher Disease, in comparison to the subject before treatment or in comparison to an untreated subject, the methods provided herein may result in one or more of the following: increase or stabilization in blood hemoglobin concentration, increase or stabilization in blood platelet count (improvement/stabilization in thrombocytopenia), increase or stabilization in bone mineral density, reduction or stabilization in hepatic or splenic volume, improvement or stabilization in Purdue Pegboard Test performance, improvement or stabilization in 36-Item Short Form Survey (SF-36) score, improvement or stabilization in Small Fiber Neuropathy Screening List (SFNSL) score, and/or reduced or stabilized biofluid chitotriosidase and/or c-c motif chemokine ligand 18 (CCL18).

In embodiments where the subject suffers from Fabry Disease, in comparison to the subject before treatment or in comparison to an untreated subject, the methods provided herein may result in one or more of the following: improvement or stabilization in right or left ventricular hypertrophy, left ventricular ejection fraction, and/or left ventricular mass index; reduction or stabilization in abnormal electrocardiogram or echocardiogram findings; improvement or stabilization in estimated glomerular filtration rate (eGFR); reduced or stabilized biofluid globotriaosylceramide (Gb3) levels; reduced or stabilized urine albumin/creatine ratio; improved or stabilized white blood cell alpha-galactosidase A (GLA) enzyme activity; and/or improvement or stabilization in 36-Item Short Form Survey (SF-36) score.

In embodiments where the subject suffers from Pompe Disease, in comparison to the subject before treatment or in comparison to an untreated subject, the methods provided herein may result in one or more of the following: reduction or stabilization in biofluid creatine kinase, glucose tetrasaccharide (GLC4) and/or hexose tetrasaccharide (HEX4); improvement or stabilization in 6 Minute Walk Test performance; improvement or stabilization in forced vital capacity (FVC); improvement or stabilization in predicted maxiumum inspiration pressure (MIP); improvement or stabilization in maximum expiratory pressure (MEP); improvement or stabilization in 36-Item Short Form Survey (SF-36) score; improvement or stabilization in Quantitative Muscle Strength Test scores and/or Manual Muscle Strength Test scores; improvement or stabilization in in Rasch-built Pompe-specific activity (R-PAct) questionnaire scores; improvement or stabilization in EuroQol 5 Dimensions 5 Levels (EQ-5D-5L) questionnaire scores; improvement or stabilization in Patient-Reported Outcomes Measurement Information System (PROMIS) scores or subscores; and/or improvement or stabilization in Gait Stairs Gower Chair (GSGC) test scores.

In embodiments where the subject suffers from Niemann Pick Disease (Types A, B, and C), in comparison to the subject before treatment or in comparison to an untreated subject, the methods provided herein may result in one or more of the following: improvement or stabilization in NPC Clinical Severity Score or subscores; improvement in saccadic eye movement parameters (Horizontal Saccadic α and Horizontal Saccadic β), improvement or stabilization in Clinician Global Impression of Change (CGIC) scores, improvement or stabilization in 36-Item Short Form Survey (SF-36) scores, and/or improvement or stabilization in EuroQol 5 Dimensions 5 Levels (EQ-5D-5L) questionnaire scores.

In embodiments where the subject suffers from GM2 Gangliosidosis (i.e., Tay-Sachs or Sandhoff Disease), in comparison to the subject before treatment or in comparison to an untreated subject, the methods provided herein may result in one or more of the following: reduction or stabilization in biofluid chitotriosidase and/or lyso-GM2 ganglioside; and/or improvement or stabilization in β-hexosaminidase A and/or B activity in blood and/or white blood cells.

The therapeutic agents may be administered in any suitable dosage and dosage form as determined by attending medical personnel. Amounts effective depend on various factors including, but not limited to, the nature of the compound (specific activity, etc.), the route of administration, the stage and severity of the disorder, the weight and general state of health of the subject, and the judgment of the prescribing physician. The active compounds are effective over a wide dosage range. The amount, timing, and dosage forms of the therapeutic agents actually administered will be determined by a physician, in the light of the above relevant circumstances.

The therapeutic agents may be administered via any route deemed appropriate by attending medical personnel, including but not limited to orally, topically, parenterally, by inhalation or spray or rectally in dosage unit formulations containing conventional non-toxic pharmaceutically acceptable carriers, adjuvants and vehicles. In some embodiments, the therapeutic agents are administered as the compositions of the disclosure detailed herein.

The subject may be any subject that can benefit from the methods of the disclosure. In one embodiment. the subject is a mammal; in a more preferred embodiment, the subject is a human.

In another aspect, the disclosure provides pharmaceutical composition, comprising:
(a) 1, 2, or all 3 of
   (i) a SORT1 inhibitor,
   (ii) a NGFR modulator, and
   (iii) a GLP-TR agonist; and
(b) a nucleic acid, including but not limited to a DNA, an expression vector, etc; wherein the nucleic acid encodes a gene therapy expression product capable of substituting for a protein deficient in a lysosomal storage disorder and/or neurological disorder; and
(c) a pharmaceutically acceptable carrier.

Non-limiting examples of SORT1 inhibitors, NGFR modulators, GLP-TR agonists, and nucleic acids for use in the compositions of the disclosure include all those disclosed herein. In one embodiment, the gene therapy product is capable of substituting for a protein deficient in a lysosomal storage disorder and/or neurological disorder as disclosed above. In one embodiment, the composition comprises a SORT1 inhibitor, such as any of those disclosed above. In another embodiment, the gene therapy product encodes PPT1, TPP1, CLN3, CLN4 (DNAJC5), CLN5, CLN6, CLN7 (MFSD8), CLN8, CLN10 (CTSD), CLN11, CLN12 (ATP13A2), CLN13 (CTSF), CLN14 (KCTD7) or functional fragment thereof. In another embodiment, the compositions may further comprise one or more additional gene therapy products that encode proteins implicated in lysosomal storage disorders, including but not limited to lysosomal alpha-glucosidase (GAA), alpha-galactosidase A (GLA), glucosylceramidase beta (GBA), acid sphingomyelinase (SMPD1), NPC intracellular cholesterol transporter 1 (NPC1), NPC intracellular cholesterol transporter 2 (NPC2), beta-galactosidase 1 (GLB1), beta-hexosamidase A (HEXA), GM2 ganglioside activator (GM2A), alpha-L iduronidase (IDUA), iduronate 2-sulfatase (IDS), N-sulfoglucosamine sulfohydrolase (SGSH), N-acetylglucosaminidase (NAGLU), heparan-alpha-glucosaminide N-acetyltransferase (HGSNAT), N-acetylglucosamine-6-sulfatase (GNS), N-acetylgalactosamine-6-sulfatase (GALNS), arylsulfatase B (ARSB), beta-glucoronidase (GUSB), hyaluronidase 1 (HYAL1), N-acetylglucosamine-1-phosphate transferase subunits alpha and beta (GNPTAB), mucolipin 1 (MCOLN1), sulfatase modifying factor 1 (SUMF1), neuraminidase 1 (NEU), cathepsin A (CTSA), alpha-mannosidase (MAN2B1), beta-mannosidase (MANBA), aspartylglucosaminidase (AGA), alpha-L-fusocidase (FUCA1), alpha-N-acetylgalactosaminidase (NAGA), arylsulfatase A (ARSA), prosaposin (PSAP), galactosylceramidase (GALC), acid ceramidase 1 (ASAH1), lipase A (LAL), cathepsin K (CTSK), cystinosin (CTNS), solute carrier family 17 member 5 (SLC17A5), lysosomal associated membrane protein-2 (LAMP2), myosin VA (MYO5A), Rab27a member RAS oncogene family (RAB27A), melanophilin (MLPH), biogenesis of lysosomal organelles complex 3 subunit 1 (HPS1), AP-2 complex subunit beta-1 (AP3B1), biogenesis of lysosomal organelles complex 2 subunit 1 (HPS3), biogenesis of lysosomal organelles complex 3 subunit 2 (HPS4), biogenesis of lysosomal organelles complex 2 subunit 2 (HPS5), biogenesis of lysosomal organelles complex 2 subunit 3 (HPS6), dystrobrevin binding protein 1 (DTNBP1), biogenesis of lysosomal organelles complex 1 subunit 3 (BLOCIS3, PLDN) adaptor related protein complex 3 subunit delta 1 (AP3D1), lysosomal trafficking regulator (LYST), or functional fragments thereof.

In another aspect, the disclosure provides a pharmaceutical composition, comprising:
(a) 2 or all 3 of:
   (i) a SORT1 inhibitor,
   (ii) a NGFR modulator, and
   (iii) a GLP-1R agonist; and
(b) a pharmaceutically acceptable carrier.

Non-limiting examples of SORT1 inhibitors, NGFR modulators, GLP-TR agonists, used in the compositions of this aspect of the disclosure include all those disclosed herein. In one embodiment, the composition comprises a SORT1 inhibitor. Exemplary embodiments of such SORT1 inhibitors are provided above, including but not limited to AF38469 or N-substituted-5-substituted pthalamic acids, AF40431 (N-[(7-hydroxy-4-methyl-2-oxo-2H-chromen-8-yl)methyl]-L-leucine) or substituted versions thereof; (S)-2-(3,5-dichlorobenzamido)-5,5-dimethylhexanoic acid or derivatives such as (S)-2-(4-chloro-TH-pyrrole-2-carboxamido)-5,5-dimethylhexanoic acid and (S)-5-5-dimethyl-2-(6-phenoxynicotinamido)hexanoic acid or substituted versions thereof; 1-benzyl-3-(tert-butyl)-1H-pyrazole-5-carboxylic acid or substituted versions thereof, SORT1 small interfering RNAs, small internally segmented interfering RNAs, short hairpin RNAs, microRNAs, and/or antisense oligonucleotides; cas9 repressors or other cas (CRISPR) repressors targeting the SORT1 locus; anti-SORT1 antibodies or antibody fragments thereof; combinations thereof; or pharmaceutically acceptable salts thereof; in particular AF38469 or a pharmaceutically acceptable salt thereof.

In one embodiment, the SORT1 inhibitor comprises AF38469, or a pharmaceutically acceptable salt thereof.

In another embodiment, the composition comprises a NGRF modulator. Exemplary embodiments of such NGRF modulators are provided above, including but not limited to LM1TA-31 and substituted versions thereof; LM11A-24 and substituted versions thereof; NGFR-binding peptides or peptide modulators of NGFR signaling, or nucleic acid constructs encoding such peptide modulators, including but not limited to peptides derived from NGFR ligands such as the pro forms of brain-derived neurotrophic factor (BDNF) and nerve growth factor (NGF); anti-NGFR antibodies or antibody fragments; combinations thereof; or pharmaceutically acceptable salts thereof; particularly LM11A-31 or pharmaceutically acceptable salt thereof.

In one embodiment, the NGRF modulator comprises LM11A-31, or a pharmaceutically acceptable salt thereof.

In another embodiment, the composition comprises a GLP-TR agonist. Exemplary embodiments of such GLP-1R agonist are provided above, including but not limited to semaglutide, exenatide, liraglutide, lixisenatide, albiglutide, dulaglutide, taspoglutide, compounds that inhibit dipeptidyl peptidase-4 (DDP-4, which degrades GLP-1) including but not limited to sitagliptin, vildagliptin, saxagliptin, linagliptin, gemigliptin, anagliptin, teneligliptin, alogliptin, trelagliptin, omarigliptin, evogliptin, gosogliptin, dutogliptin, berberine; compounds that elicit GLP-1 secretion including metformin; GLP-1R agonist peptides, or nucleic acid constructs encoding such agonist peptides, including but not limited to peptides consisting of at least eight contiguous amino acids of GLP-1R ligands such as glucagon-like peptide-1 (GLP-1, human amino acid sequence: HDEFERHAEGTFTSDVSSYLEGQAAKEFIAWLVKGRG; SEQ ID NO:1) and exendin-4 (amino acid sequence: HGEGTFTSDLSKQMEEEAVRLFIEWLKNGGPSSGAPPPS; SEQ ID NO:2), or mutated versions thereof; anti-GLP-1R agonist antibodies or antibody fragments; combinations thereof; or pharmaceutically acceptable salts thereof.

In one embodiment, the GLP-TR agonist comprises semaglutide, or a pharmaceutically acceptable salt thereof.

In one embodiment, the composition comprises AF38469 or a pharmaceutically acceptable salt thereof, and LM11A-31 or a pharmaceutically acceptable salt thereof. In one embodiment, the composition further comprises semaglutide or a pharmaceutically acceptable salt thereof.

In another embodiment, the composition comprises AF38469 or a pharmaceutically acceptable salt thereof, and semaglutide or a pharmaceutically acceptable salt thereof.

In another embodiment, the composition comprises LM11A-31 or a pharmaceutically acceptable salt thereof, and semaglutide or a pharmaceutically acceptable salt thereof.

The term “pharmaceutical composition” is used in its widest sense, encompassing all pharmaceutically applicable compositions containing the recited therapeutic agents, and optional carriers, adjuvants, constituents etc. The term “pharmaceutical composition” also encompasses a composition comprising the recited therapeutic agents in the form of derivatives or pro-drugs, such as pharmaceutically acceptable salts and esters. The manufacture of pharmaceutical compositions for different routes of administration falls within the capabilities of a person skilled in medicinal chemistry.

Exemplary pharmaceutically acceptable salts include salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, HOOC (CH₂)ₙ COOH where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

The compositions may be made up in a solid form (including granules, powders or suppositories) or in a liquid form (e.g., solutions, suspensions, or emulsions). The compositions may be applied in a variety of solutions and may be subjected to conventional pharmaceutical operations such as sterilization and/or may contain conventional adjuvants, such as preservatives, stabilizers, wetting agents, emulsifiers, buffers etc.

The compositions may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, intracerebroventricular, or intrathecal injection or infusion techniques and the like. In addition, a pharmaceutical formulation is provided comprising a compound of the disclosure and a pharmaceutically acceptable carrier. One or more compounds of the disclosure may be present in association with one or more non-toxic pharmaceutically acceptable carriers and/or diluents and/or adjuvants, and if desired other active ingredients. The pharmaceutical compositions containing therapeutic agents of the disclosure may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide palatable preparations. Tablets contain the therapeutic agents in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the therapeutic agents are mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the therapeutic agents in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the therapeutic agents in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the therapeutic agents in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the disclosure may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The pharmaceutical compositions of the present disclosure may also be administered in the form of suppositories, e.g., for rectal administration of the drug. These compositions can be prepared by mixing the therapeutic agents with a suitable non-irritating excipient that is solid at ordinary temperatures but liquid at the rectal temperature and will therefore melt in the rectum to release the drug. Such materials include cocoa butter and polyethylene glycols.

The pharmaceutical compositions of the present disclosure may be administered parenterally in a sterile medium. The therapeutic agents, depending on the vehicle and concentration used, can either be suspended or dissolved in the vehicle. Advantageously, adjuvants such as local anesthetics, preservatives and buffering agents can be dissolved in the vehicle.

Formulations for intravenous, intrathecal, or intracerebroventricular administration may include contrast agents (e.g. iohexol), surfactants (e.g. Pluronic F-68), sugars (e.g. sorbitol) or other excipients that prevent aggregation, provide cryoprotection, or enhance stability of active compounds.

### Definitions

Terms used herein may be preceded and/or followed by a single dash, “—”, or a double dash, “═”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” (i.e., the attachment is via the last portion of the name) unless a dash indicates otherwise. For example, C₁-C₆alkoxycarbonyloxy and —OC(O)C₁-C₆alkyl indicate the same functionality; similarly arylalkyl and -alkylaryl indicate the same functionality.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, and —CH₂CH(CH₂CH₃)CH₂—.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thioxo groups. Representative examples of the bicyclic aryls include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl. 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 2H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-7-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or thioxo.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted with one or two groups which are independently oxo or thioxo. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thioxo.

The term “halo” or “halogen” as used herein, means —Cl, —Br, —I or —F.

The terms “haloalkyl” and “haloalkoxy” refer to an alkyl or alkoxy group, as the case may be, which is substituted with one or more halogen atoms.

The term “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thioxo. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a benzo ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, 2,3-dihydrothieno[3,4-b][1,4]dioxan-5-yl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thioxo.

The terms “heterocyclyl” and “heterocycloalkyl” as used herein, mean a monocyclic heterocycle or a bicyclic heterocycle. The monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl. 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl. oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl. tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to either a phenyl, a monocyclic cycloalkyl, a monocyclic cycloalkenyl, a monocyclic heterocycle, or a monocyclic heteroaryl. The bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-TH-indolyl, and octahydrobenzofuranyl. Heterocyclyl groups are optionally substituted with one or two groups which are independently oxo or thioxo. In certain embodiments, the bicyclic heterocyclyl is a 5 or 6 membered monocyclic heterocyclyl ring fused to phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thioxo.

The term “saturated” as used herein means the referenced chemical structure does not contain any multiple carbon-carbon bonds. For example, a saturated cycloalkyl group as defined herein includes cyclohexyl, cyclopropyl, and the like.

The term “substituted”, as used herein, means that a hydrogen radical of the designated moiety is replaced with the radical of a specified substituent, provided that the substitution results in a stable or chemically feasible compound. The term “substitutable”. when used in reference to a designated atom, means that attached to the atom is a hydrogen radical, which can be replaced with the radical of a suitable substituent.

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

The term “unsaturated” as used herein means the referenced chemical structure contains at least one multiple carbon-carbon bond, but is not aromatic. For example, a unsaturated cycloalkyl group as defined herein includes cyclohexenyl, cyclopentenyl, cyclohexadienyl, and the like.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Pharmaceutically acceptable salt” refers to both acid and base addition salts.

### Example 1

NCL Proteins Interact with SORT1-Related Machinery

As part of our investigation into the basic biological functions of the transmembrane NCL proteins CLN3, CLN6, and CLN8, we performed proximity-dependent biotinylation screens for both proteins to identify protein interactors in living cells. This technique employs a fusion protein of the bait protein of interest (i.e. CLN3, CLN6, or CLN8) fused to a promiscuous biotin ligase which covalently links biotin molecules to proximal interactors in the cell. This biotin tag then enables high-affinity capture and subsequent mass spectrometry-based identification of interactors. Interactor data for CLN3, CLN6, CLN8 was highly enriched for SORT1 interactors including lysosomal cargoes, AP-1 adapter proteins, and retromer subunits, suggesting that all three of these NCL proteins may have important roles in SORT1-related pathways.

TABLE 1
SORT1-related proteins identified in BioID screens for CLN3 and CLN8.
BioID protein-protein interaction screens were performed for CLN3
and CLN8 using Neuro 2A cell lines expressing CLN3- or CLN8-BirA.
A number of critical SORT1-related interactors were identified.

| Protein | Source | |
|---|---|---|
| Interactor | Screen | Known Function(s) |
| VPS35 | CLN3, CLN8 | Retromer complex subunit |
| VPS26A | CLN3, CLN8 | Retromer complex subunit |
| VPS26B | CLN3 | Retromer complex subunit |
| VPS29 | CLN8 | Retromer complex subunit |
| SNX5 | CLN8 | Retromer complex subunit |
| SNX1 | CLN3 | Retromer complex subunit |
| SNX2 | CLN3 | Retromer complex subunit |
| TBC1D5 | CLN3 | GTPase activating protein (GAP) for RAB7A, which recruits |
| | | retromer to membranes |
| TBC1D15 | CLN3, CLN8 | GTPase activating protein (GAP) for RAB7A, which recruits |
| | | retromer to membranes |
| AP1G1 | CLN3, CLN8 | Cytosolic SORT1 partner mediating intracellular trafficking |
| AP1M1 | CLN3, CLN8 | Cytosolic SORT1 partner mediating intracellular trafficking |
| AP1B1 | CLN3, CLN8 | Cytosolic SORT1 partner mediating intracellular trafficking |
| AP1S1 | CLN8 | Cytosolic SORT1 partner mediating intracellular trafficking |
| CTSD | CLN6, CLN8 | Lysosomal enzyme trafficked by SORT1 |
| NGFR | CLN8 | SORT1 coreceptor for proBDNF signaling |

Sortilin (SORT1) is a transmembrane receptor with diverse cellular functions. At the cell surface, SORT1 acts as a receptor for Neurotensin, which it scavenges and brings to the lysosome for degradation. SORT1 also acts as a coreceptor for the pro form of Brain Derived Neurotrophic Factor (proBDNF). While SORT1 is not believed to have catalytic activity, it appears to regulate the propagation of neurotoxic signals generated by proBDNF binding to the p75 neurotrophin receptor (NGFR, also known as p75NTR). SORT1 also acts as a modulator of intracellular sorting, regulating the sorting of diverse cargoes including amyloid proteins, glucose transporters, and, importantly, lysosomal machinery. For this function, SORT1 binds lysosomal cargoes in the trans-Golgi network through its N-terminal ligand-binding domain, while its C-terminal cytosolic domain recruits adaptor proteins (e.g. adapter protein 1, AP-1) that are responsible for trafficking to endosomes and lysosomes. SORT1 releases lysosomal cargoes in the acidic environment of the lysosome, and then binds the retromer complex for recycling back to the Golgi network for additional rounds of trafficking [6].

We subsequently found that SORT1 inhibition activated lysosomal biogenesis through novel activity consisting of activation of key transcription factors transcription factor EB (TFEB) and transcription factor E3 (TFE3). In animal models of two lysosomal storage disorders, we found that SORT1 inhibition leads to beneficial impacts on lysosomal function, reductions in lysosomal storage, reductions in neuroinflammation, rescue of behavioral abnormalities (i.e. tremors), and restoration of normal body weight.

### Example 2

TABLE 2
Abbreviations and Specialist Terms

| Abbreviation or | |
|---|---|
| specialist term | Explanation |
| AAALAC | Association for Assessment and Accreditation of Laboratory Animal Care |
| ASM | autofluorescent storage material |
| C57BL/6J | A strain of laboratory mouse available from Jax Labs |
| CD68 | Cluster of Differentiation 68. When used as an antibody, a marker of activated |
| | microglia |
| CFR | code of federal regulations |
| CLEAR | Coordinated Lysosomal Expression and Regulation, a gene network that regulates |
| | lysosomal biogenesis and function |
| CLN | Ceroid Lipofuscinosis Neuronal |
| CLN2-Batten | Batten disease caused by biallelic mutations in CLN2 |
| CLN3-Batten | Batten disease caused by biallelic mutations in CLN3 |
| CLN6-Batten | Batten disease caused by biallelic mutations in CLN6 |
| Cln1^{R151X} | A mouse model with homozygous recessive pathogenic mutations in Cln1, used as |
| | a model for CLN1-Batten disease |
| Cln2^{R207X} | A mouse model with homozygous recessive pathogenic mutations in Cln2, used as |
| | a model for CLN2-Batten disease |
| Cln3^{Δex7/8} | A mouse model with homozygous recessive pathogenic mutations in Cln3, used as |
| | a model for CLN3-Batten disease |
| Cln6^{nclf} | A mouse model with homozygous recessive pathogenic mutations in Cln6, used as |
| | a model for CLN6-Batten disease |
| Cln8^{mnd} | A mouse model with homozygous recessive pathogenic mutations in Cln8, used as |
| | a model for CLN8-Batten disease |
| Cln11^{-/-} | A mouse model with homozygous recessive pathogenic mutations in Cln11, used |
| | as a model for CLN11-Batten disease |
| CNS | central nervous system |
| CO₂ | carbon dioxide |
| CX7 | CellInsight ™ CX7 High-Content Screening Platform |
| DAB | 3,3-Diaminobenzidine |
| DAPI | 4′,6-diamidino-2-phenylindole |
| DEG(s) | differentially expressed gene(s) |
| DIV3/5/7 | days in vitro 3/5/7 |
| DMSO | Dimethylsulfoxide, used as a vehicle for drug solubility |
| DNase | Deoxyribonuclease |
| E15.5 | Embryonic day 15.5 |
| ER | Endoplasmic reticulum |
| FDA | United States Food and Drug Administration |
| GFAP | Glial fibrillary acidic protein, a marker of astrocytes |
| GFP | green fluorescent protein |
| GMP | good manufacturing practices |
| Hz | hertz (s⁻¹), unit of frequency |
| IACUC | Institutional Animal Care and Use Committee |
| IHC | immunohistochemistry |
| Lamp1 | Lysosomal-associated membrane protein 1, a marker of lysosomes. |
| LSDs | lysosomal storage disorders |
| M6P | Mannose 6-Phosphate |
| MEFs | mouse embryonic fibroblasts |
| N2As | mouse neuroblastoma cell line |
| NCL | neuronal ceroid lipofuscinosis |
| NIH | National Institutes of Health |
| nM/μM | nanomolar/micromolar, Measurement of drug concentration |
| P21 | postnatal day 21 |
| PBS | phosphate buffered saline |
| PNCs | Primary neuronal cells |
| PPT1 | palmitoyl-protein thioesterase 1, the protein product of the CLN1 gene |
| S1BF | Primary somatosensory cortex barrel field 1 |
| SORT1 | sortilin |
| SubC | Mitochondrial ATP synthase subunit C, a lysosomal storage substrate in multiple |
| | forms of Batten disease |
| TBS | tris-buffered saline |
| TBS-T | tris-buffered saline- with 0.3% Triton X-100 |
| TFE3 | Transcription Factor Binding to IGHM Enhancer 3 |
| TFEB | Transcription Factor EB |
| TPP1 | tripeptidyl peptidase 1 |
| USDA | US Department of Agriculture |
| μg | Microgram. Measurement of drug concentration |
| VPM/VPL | ventral posteromedial nucleus/ventral posterolateral nucleus of the thalamus |
| VPS10 | vacuolar protein sorting 10 protein |
| WT | wild type |

Batten disease (as referred to as neuronal ceroid lipofuscinosis [NCLs]) is a family of primarily autosomal recessive, predominantly pediatric, phenotypically similar lysosomal storage disorders that are rare (incidence 2-4/100,000 births) and universally fatal. Batten disease is caused by mutations in one of at least 13 known ceroid lipofuscinosis, neuronal (CLN) genes that encode for a variety of extralysosomal and lysosomal proteins, many of which have unknown function.

We tested AF38469, an orally available, specific small molecule inhibitor of sortilin, in in vitro models of CLN1, CLN2, CLN3, CLN6, CLN8, and CLN11-Batten disease and in vivo in mouse models of CLN2 and CLN3 Batten disease. Using the in vitro models, we showed that AF38469 reduced lysosomal pathology and improved cellular health. These effects appear to be mediated in part by activation of TFEB and TFE3, master transcriptional regulators of lysosomal biogenesis and function. Using the in vivo CLN2 and CLN3 Batten disease mouse models, we show that treatment with AF38469 reduced pathological and behavioral hallmarks of disease. Collectively, our results demonstrate that AF3846 is a potent and effective modulator of lysosomal function that exhibits in vivo efficacy in two lysosomal storage disorders with vastly different etiology, suggesting that sortilin inhibition could have broad applicability as a therapeutic strategy for disorders characterized by lysosomal dysfunction.

### Study Groups

In vitro groups consisted of 3 cell culture wells per treatment per genotype. Mouse embryonic fibroblasts (MEFs) and primary cortical neurons from 6 wild type and mutant lines were utilized, (WT, Cln1^{R151X}, Cln2^{R207X}, Cln3^{Δex7/8}, Cln6^{nclf}, Cln8^{mnd}, and Cln11^{-/-}). In vivo groups consisted of 7-8 mice comprised of mixed and balanced sexes, age-matched across all groups. Three mouse strains were utilized, C57BL/6J Wild Type (WT), Cln2^{R207X} point mutant mice, and Cln3^{Δex7/8} knock-in mutant mice.

### Methods

Cells were dosed with AF38469 on DIV3 and DIV5; on DIV7, cells were collected and analyzed. Mice were dosed with vehicle or AF38469-containing drinking water starting at wean (approximately P21). Cln2^{R207X} mice were sacrificed for histological and biochemical analyses at 11 weeks of age, and Cln3^{Δex7/8} mice were sacrificed at 16 weeks of age. Tissue was processed using standard techniques.

### Study Materials

### Test Article(s)

In in vitro models, AF38469 (VulcanChem, catalog #VC1034757) was tested in concentrations of 40 nM, 400 nM, and 4 μM in 1% DMSO. Vehicle treated cells were dosed with 1% DMSO vehicle. In in vivo models, AF38469 (VulcanChem, catalog #VC1034757) was tested in concentrations of (78.125 μg/ml, 3.125 μg/ml, 0.3125 μg/ml or 0.03125 μg/ml equating approximately to 250 μg/mouse/day, 10 μg/mouse/day, 1 μg/mouse/day, and 0.1 μg/mouse/day, respectively, given a mouse's daily water intake of approximately 3.2 ml per day) dosed in drinking water with a concentration of 0.2408% DMSO. Vehicle treated animals were dosed with drinking water containing 0.2408% DMSO. AF38469 and vehicle water was prepared weekly. AF38469 was utilized at ≥98% purity.

### Test System

All animals are maintained on a C57BL/6J background and housed under identical conditions. Wild type animals lacked these mutations. All animal studies are conducted in an AAALAC accredited facility under NIH guidelines and approved by Sanford IACUC (USDA license 46-R-0009). Cln1^{R151X} point mutant mice (The Jackson Laboratory #026197) replicate a nonsense mutation commonly seen in CLN1 patients. Cln2^{R207X} point mutant mice (The Jackson Laboratory #030696) replicate a nonsense mutation seen in CLN2 patients. Cln3^{Δex7/8} knock-in mutant mice (The Jackson Laboratory #017895) replicate a common mutation seen in CLN3 patients in which deletions of exons 7 and 8 lead to a premature stop codon. Cln6^{nclf} mice (The Jackson Laboratory #003605) replicate a common mutation seen in CLN6 patients, in which an insertion leads to a premature stop codon. Cln8^{mnd} mutant mice (The Jackson Laboratory #001612) replicate a spontaneous mutation commonly found in CLN8 patients. C57BL/6J (Wild type (WT)) animals lacked these mutations. Mouse embryonic fibroblasts (MEFs) and primary cortical neurons (PNCs) are collected from WT or mutant mice on embryonic day 15.5, and primary cell cultures are generated for in vitro testing. Other cell lines tested include Cln11^{-/-} (Grn−/−) MEFS which were generated from Cln11^{-/-} mice and gifted from the Kukar lab. Cln2^{R270X} and Cln3^{Δex7/8} mice were dosed through drinking water starting at wean.

### Experimental Procedures

### Study Groups

In vitro studies:
   WT MEFs in triplicate treated with vehicle, 40 nM AF38469, 400 nM AF38469, 4 μM AF38469 for LysoTracker™, and ASM analyses.
   Cln1^{R151X} MEFs in triplicate treated with vehicle, 40 nM AF38469, 400 nM AF38469, 4 μM AF38469 for LysoTracker™, and ASM analyses.
   Cln2^{R207X} MEFs in triplicate treated with vehicle, 40 nM AF38469, 400 nM AF38469, 4 μM AF38469 for LysoTracker™, and ASM analyses.
   Cln3^{Δex7/8} MEFs in triplicate treated with vehicle, 40 nM AF38469, 400 nM AF38469, 4 μM AF38469 for LysoTracker™, and ASM analyses.
   Cln6^{nclf} MEFs in triplicate treated with vehicle, 40 nM AF38469, 400 nM AF38469, 4 μM AF38469 for LysoTracker™, and ASM analyses.
   Cln8^{mnd} MEFs in triplicate treated with vehicle, 40 nM AF38469, 400 nM AF38469, 4 μM AF38469 for LysoTracker™, and ASM analyses.
   Cln11^{-/-} MEFs in triplicate treated with vehicle, 40 nM AF38469, 400 nM AF38469, 4 μM AF38469 for LysoTracker™, and ASM analyses.
   WT PNCs in triplicate treated with vehicle, 40 nM AF38469, 400 nM AF38469, 4 μM AF38469 for LysoTracker™, and ASM analyses.
   Cln1^{R151X} PNCs in triplicate treated with vehicle, 40 nM AF38469, 400 nM AF38469, 4 μM AF38469 for LysoTracker™, and ASM analyses.
   Cln2^{R207X} PNCs in triplicate treated with vehicle, 40 nM AF38469, 400 nM AF38469, 4 μM AF38469 for LysoTracker™, and ASM analyses.
   Cln3^{Δex7/8} PNCs in triplicate treated with vehicle, 40 nM AF38469, 400 nM AF38469, 4 μM AF38469 for LysoTracker™, and ASM analyses.
   Cln6^{nclf} PNCs in triplicate treated with vehicle, 40 nM AF38469, 400 nM AF38469, 4 μM AF38469 for LysoTracker™, and ASM analyses.
   Cln8^{mnd} PNCs in triplicate treated with vehicle, 40 nM AF38469, 400 nM AF38469, 4 μM AF38469 for LysoTracker™, and ASM analyses.
In vivo higher dose (3.125 μg/ml and 78.125 μg/ml) study in Cln2^{R207X} mice
   8 (4 per sex) WT mice were treated with vehicle (0.2408% DMSO) from wean until 11 weeks of age as a control for the Cln2^{R207X} group
   8 (4 per sex) WT mice were treated with 3.125 μg/ml AF38469 from wean until 11 weeks of age as a control for the Cln2^{R207X} group
   8 (4 per sex) WT mice were treated with 78.125 μg/ml AF38469 from wean until 11 weeks of age as a control for the Cln2^{R207X} group
   7 (3 male and 4 female) Cln2^{R207X} mice were treated with vehicle (0.2408% DMSO) from wean until 11 weeks of age
   8 (4 per sex) Cln2^{R207X} mice were treated with 3.125 μg/ml AF38469 from wean until 11 weeks of age
   8 (4 per sex) Cln2^{R207X} mice were treated with 78.125 μg/ml AF38469 from wean until 11 weeks of age
In vivo higher dose study in Cln3^{Δex7/8} mice
   8 (4 per sex) WT mice were treated with vehicle (0.2408% DMSO) from wean until 16 weeks of age as a control for the Cln3^{Δex7/8} group
   8 (4 per sex) WT mice were treated with 3.125 μg/ml AF38469 from until 16 weeks of age as a control for the Cln3^{Δex7/8} group
   8 (4 per sex) WT mice were treated with 78.125 μg/ml AF38469 from wean until 16 weeks of age as a control for the Cln3^{Δex7/8} group
   8 (4 per sex) Cln3^{Δex7/8} mice were treated with vehicle (0.2408% DMSO) from wean until 16 weeks of age
   8 (4 per sex) Cln3^{Δex7/8} mice were treated with 3.125 μg AF38469 from wean until 16 weeks of age
   8 (4 per sex) Cln3^{Δex7/8} mice were treated with 78.125 μg/ml AF38469 from wean until 16 weeks of age
In vivo lower dose study (0.03125 μg/ml) study in Cln2^{R207X} mice
   8 (4 per sex) WT mice were treated with vehicle (0.2408% DMSO) from wean until 11 weeks of age as a control for the Cln2^{R207X} group
   7 (4 male and 3 female) WT mice were treated with 0.03125 μg/ml AF38469 from wean until 11 weeks of age as a control for the Cln2^{R207X} group
   7 (3 male and 4 female) WT mice were treated with 0.3125 μg/ml AF38469 from wean until 11 weeks of age as a control for the Cln2^{R207X} group
   8 (4 per sex) Cln2^{R207X} mice were treated with vehicle (0.2408% DMSO) from wean until 11 weeks of age
   8 (4 per sex) Cln2^{R207X} mice were treated with 0.03125 μg/ml AF38469 from wean until 11 weeks of age
   8 (4 per sex) Cln2^{R207X} mice were treated with 0.3125 μg/ml AF38469 from wean until 11 weeks of age
In vivo lower dose study (0.03125 μg/ml and 0.3125 μg/ml) study in Cln3^{Δex7/8} mice
   8 (4 per sex) WT mice were treated with vehicle (0.2408% DMSO) from wean until 16 weeks of age as a control for the Cln3^{Δex7/8} group
   8 (4 per sex) WT mice were treated with 0.03125 μg/ml AF38469 from until 16 weeks of age as a control for the Cln3^{Δex7/8} group
   8 (4 per sex) WT mice were treated with 0.3125 μg/ml AF38469 from wean until 16 weeks of age as a control for the Cln3^{Δex7/8} group
   8 (4 per sex) Cln3^{Δex7/8} mice were treated with vehicle (0.2408% DMSO) from wean until 16 weeks of age
   8 (4 per sex) Cln3^{Δex7/8} mice were treated with 0.03125 μg/ml AF38469 from wean until 16 weeks of age
   8 (4 per sex) Cln3^{Δex7/8} mice were treated with 0.3125 μg/ml AF38469 from wean until 16 weeks of age

### Experimental Details

Specific experimental details are included in figure legends. In general, after collection and preparation, MEFs and PNCs were dosed with vehicle or AF38469 (40 nM, 400 nM. or 4 μM) on DIV3 and DIV5. On DIV7, cells were treated with the following dyes: Hoechst 33342 (1:1000 dilution), LysoTrackerT™ Red DND-99 (1:10,000 dilution), fixed, and imaged. Mice were given vehicle or AF38469 (78.125 μg/ml, 3.125 μg/ml, 0.3125 μg/ml or 0.03125 μg/ml equating approximately to 250 μg/mouse/day, 10 μg/mouse/day, 1 μg/mouse/day, and 0.03125 μg/mouse/day, respectively, given a mouse's daily water intake of approximately 3.2 ml per day) in drinking water beginning at wean. Body weight was measured bi-weekly, and a force actimeter test was performed on Cln2^{R207X} mice at 10 weeks of age. Body weight was measured every month for Cln3^{Δex7/8} mice. At 11 and 16 weeks of age, respectively, Cln2^{R207X} and Cln3^{Δex7/8} mice along with age matched wild type control mice were CO₂ euthanized and intracardially perfused with PBS. Brains were collected and placed on a 1 mm sagittal brain block. Brains were sliced at the midline and 3 mm right of the midline. One hemisphere was fixed in 4% PFA and sectioned at 50 microns on a vibratome. Immunohistochemistry was performed on free-floating sections. For mitochondrial ATP synthase subunit C (SubC), glial fibrillary acidic protein (GFAP), and cluster of differentiation 68 (CD68) immunolabeling, primary antibodies included anti-SubC (Abcam, ab181243; 1:1000), anti-GFAP (Dako, Z0334; 1:5000), anti-CD68 (AbD Serotec, MCA1957; 1:2000), and secondary antibodies included anti-rabbit biotinylated (Vector Labs, BA-1000 1:1000) and anti-rat (Vector Labs, BA-9400 1:1000). Coverslips were mounted on slides using antifade-mounting media and were stored in the dark before imaging. Sections were imaged and analyzed using an Aperio Digital Pathology Slide Scanner (AT2) and associated software. Images were extracted from S1BF of the somatosensory cortex and the VPM/VPL of the thalamus, with multiple images taken of multiple tissues from each animal.

### Data Analysis

Percent area of fluorescent signal and immunoreactivity were quantified using a threshold analysis in ImageJ™. Statistical analyses were performed using GraphPad Prism™ (v8.4.3; San Diego, CA). Details of the specific tests are noted in the figure legends. In general, for in vitro analysis, two-way ANOVA was employed with Scheffe post hoc for multiple comparisons. In general, for in vivo analysis, one-way ANOVA was employed with Dunnett's post hoc for multiple comparisons. and outliers were removed with the ROUT method, Q=1%. *p<0.05, **p<0.01, ***p<0.001, ****p<0.0001

### Interpretation of Results

An increase in percent area or total area of positive autofluorescent storage material (ASM) signal in untreated NCL MEFs or PNCs relative to age-matched, wild type MEFs or PNCs is considered an increase in the accumulation of autofluorescent storage material, reflecting disease-related lysosomal dysfunction.
A decrease in percent area or total area of positive autofluorescent storage material signal in AF38469 treated MEFs and PNCs relative to age-matched, vehicle treated MEFs and PNCs is considered a reduction in the accumulation of autofluorescent storage material and thus a decrease in disease-related lysosomal dysfunction.
LysoTracker™ is an acidophilic dye that labels lysosomes and accumulation increases with an increase in lysosome size or number. Cell and mouse models of lysosomal storage disorders often have increased accumulation of lysosomes and therefore have a stronger LysoTracker™ signal. Drug-related changes in LysoTracker™ signal could be due to reduction of lysosomal pathology, changes in lysosomal biogenesis, or both, and are thus best interpreted alongside other measures of lysosomal function such as autofluorescent storage material.
An increase in percent area or total area of positive SubC in vehicle treated Batten disease mice relative to age-matched vehicle treated wild type mice is considered an increase in the accumulation of mitochondrial ATP Synthase Subunit C, reflecting disease-related lysosomal dysfunction.
A decrease in Percent area or Total area of positive SubC in AF38469 treated mice relative to vehicle treated Batten disease mice is considered a reduction in the accumulation of mitochondrial ATP Synthase Subunit C and thus a reduction in disease-related lysosomal dysfunction.
An increase in Percent area or Total area of positive CD68 staining in vehicle treated Batten disease mice relative to age-matched vehicle treated wild type mice is considered an increase microgliosis and neuroinflammation.
A decrease in Percent area or Total area of positive CD68 staining in AF38469 treated mice relative to vehicle treated Batten disease mice is considered a reduction in the accumulation of microgliosis.
An increase in Percent area or Total area of positive GFAP staining in vehicle treated Batten disease mice relative to age-matched vehicle treated wild type mice is considered an increase in the accumulation of astrogliosis and neuroinflammation.
A decrease in Percent area or Total area of positive GFAP staining in AF38469 treated mice relative to vehicle treated Batten disease mice is considered a reduction in the accumulation of astrogliosis.

### Results

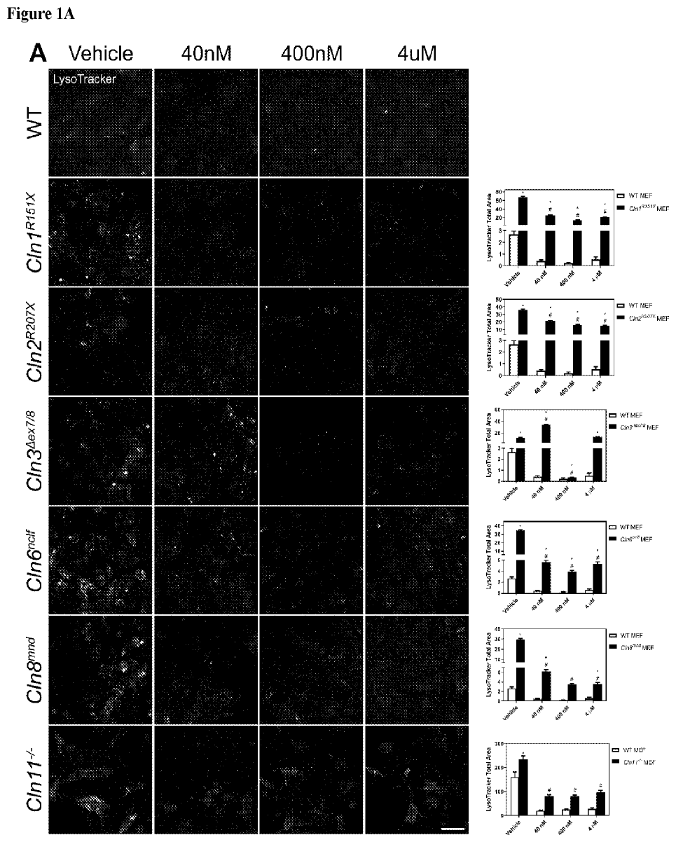

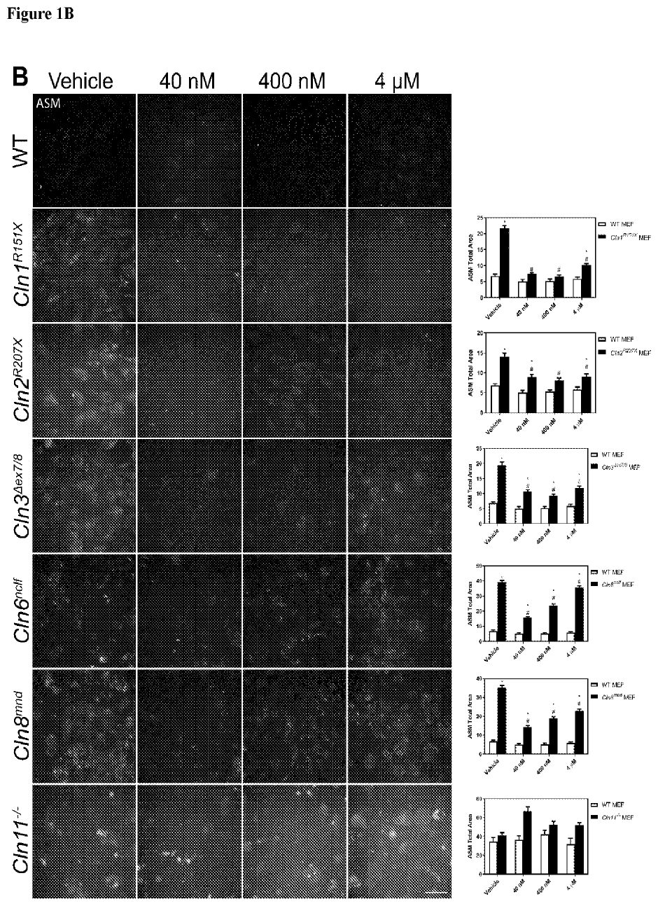

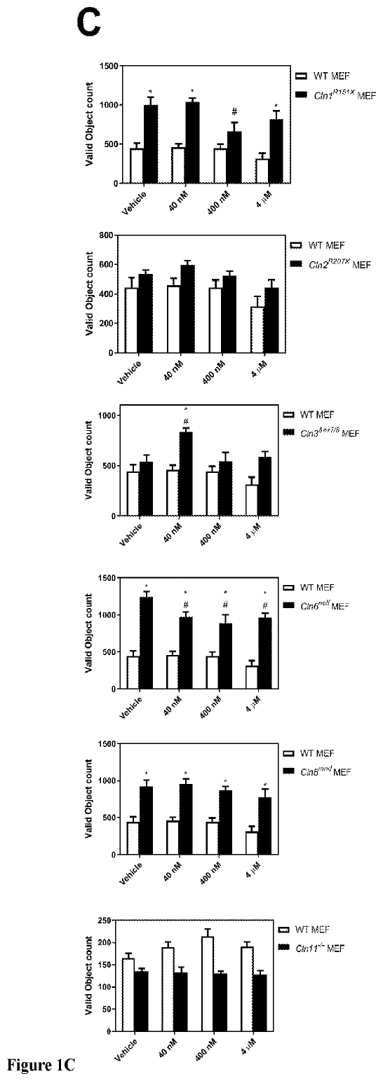

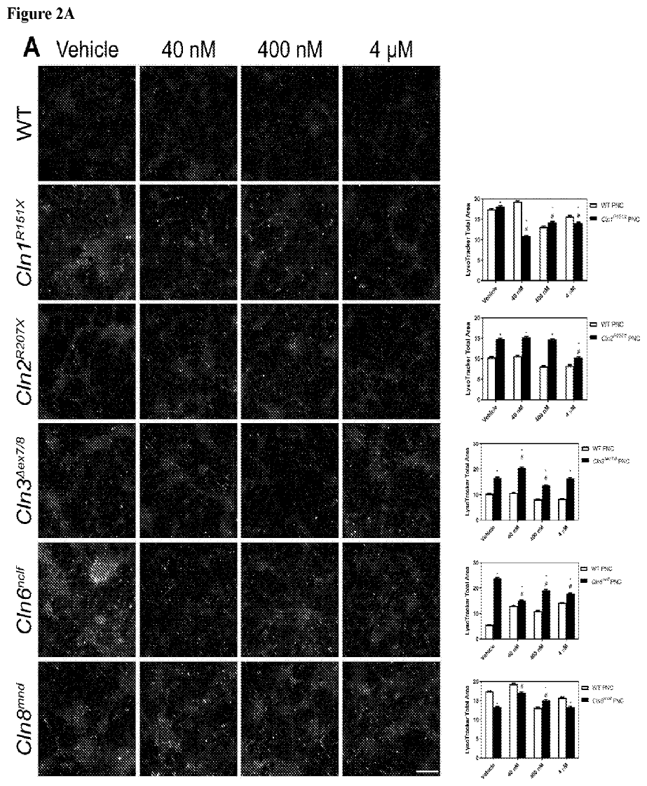

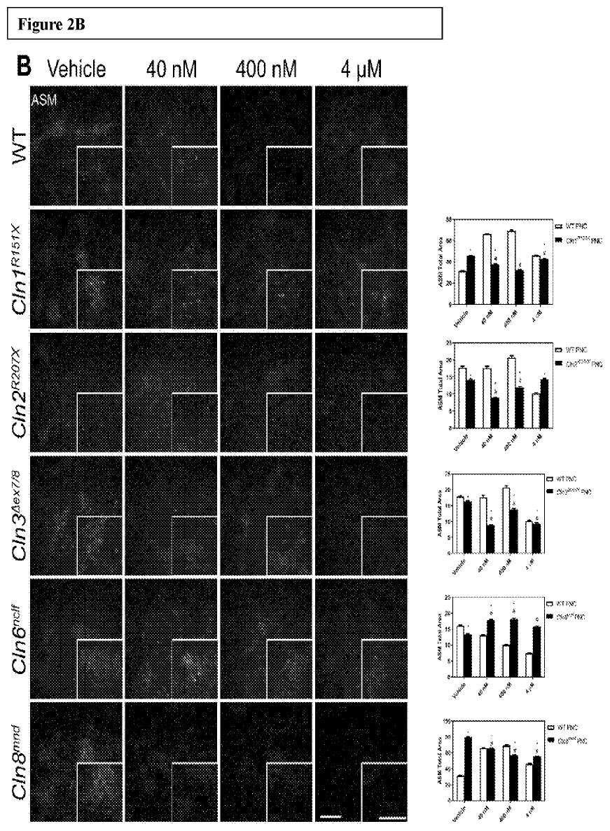

Treatment with AF38469 reduces pathology in Batten disease mouse embryonic fibroblasts. Wild type (WT) and mutant cells were treated with vehicle or AF38469 (40 nM, 400 nM, 4 μM). Cells were plated on day 0 and dosed with drug-containing media on DIV3 and DIV5. Analysis occurred on DIV7, and data is shown in FIG. 1. Concentrations of drug are shown on the x-axis above each image column; genotypes are shown to the left of each row. The y-axis of the bar graphs shows “Total Area” units which indicate % of the total cell area. The y-axis of the bar graphs in (D) shows “Valid Object Count” which reflects number of cells. Increasing concentrations of AF38469 are shown along the x-axis. Two-way ANOVA with a Scheffe post-hoc test *=significantly (p<0.05) different from wild type (WT) vehicle. #=significantly (p<0.05) different from NCL vehicle. This data indicated that SORT1 inhibition through the use of AF38469 reduced lysosomal pathology in a wide range of NCL MEFs without reducing cellular viability.

Treatment with AF38469 reduces pathology in Batten disease primary neuronal cultures. Wild type (WT) and mutant cells were treated with vehicle or AF38469 (40 nM, 400 nM, 4 μM). Cells were plated on day 0 and dosed with drug-containing media on DIV3 and DIV5. Analysis occurred on DIV7, and data is shown in FIG. 2 Concentrations of drug are shown on the x-axis above each image column; genotypes are shown to the left of each row. The y-axis of the bar graphs shows “Total Area” units which indicate % of the total cell area. The y-axis of the bar graphs in (D) shows “Valid Object Count” which reflects number of cells. Increasing concentrations of AF38469 are shown along the x-axis. Two-way ANOVA with a Scheffe post-hoc test *=significantly (p<0.05) different from wild type (WT) vehicle. #=significantly (p<0.05) different from NCL vehicle. This data indicated that SORT1 inhibition through the use of AF38469 reduced lysosomal pathology in a wide range of NCL PNCs without reducing cellular viability.

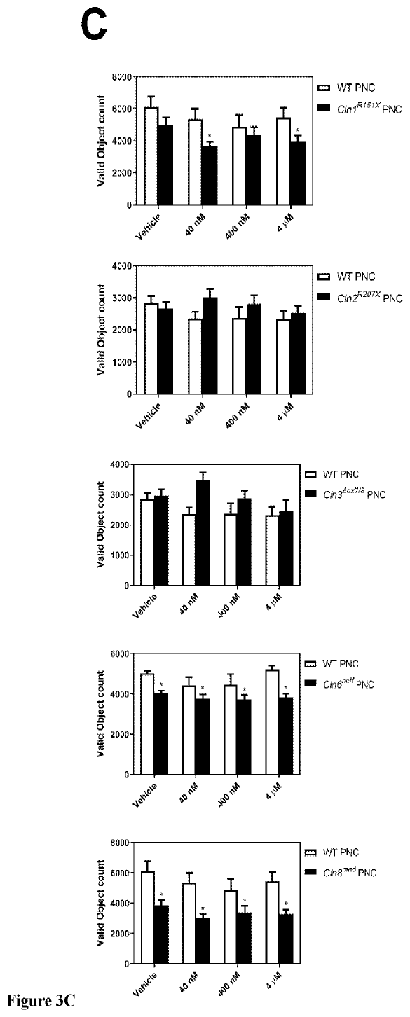

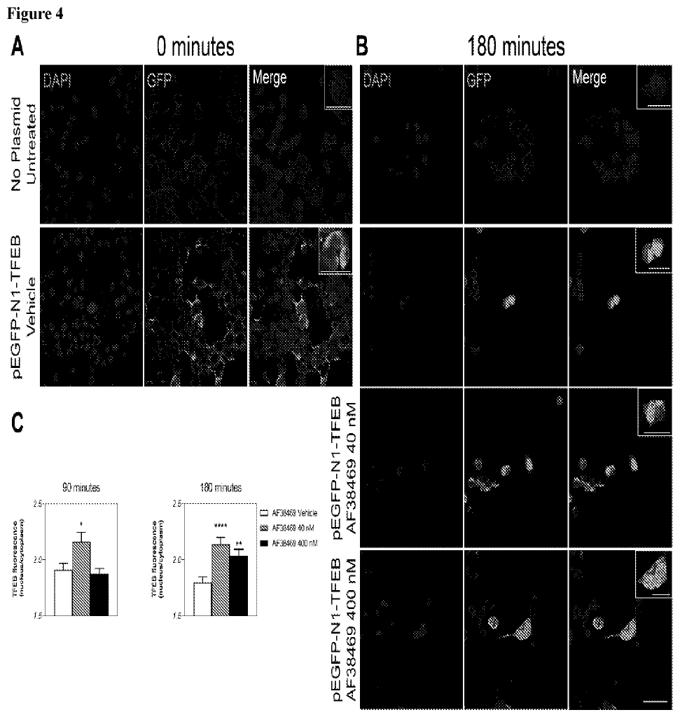

Since AF38469 improves lysosomal health and function across diverse cell models of lysosomal storage disorders, we asked whether the drug could be activating transcriptional networks that regulate lysosomal function. The transcription factors TFEB and TFE3 are master regulators of lysosomal biogenesis and function. We hypothesized that AF38469 could exert benefits by modulating the nuclear translocation of TFEB and/or TFE3. We tested this in an in vitro system in which GFP fusions of TFEB or TFE3 are expressed and monitored with live imaging (FIGS. 3-4). Acute treatment with AF38469 stimulated the nuclear translocation of both TFEB and TFE3. We also investigated whether downstream targets of these transcription factors are activated following exposure to AF38469 and found that drug treatment stimulated increased expression for an abundance of TFEB targets (FIG. 5). We also measured activity levels of two lysosomal enzymes regulated by TFEB, PPT1 and TPP1, and found that AF38469 activity increased the overall levels of activity for both enzymes in cell lysates (FIG. 6).

Treatment with AF38469 stimulates TFEB nuclear translocation as monitored via time course study. See FIG. 3 for data. For (A) and (B), wild type neuro 2A rat neuroblastoma (N2A) cells were plated and transfected with a pEGFP-N1-TFEB plasmid for a 24-hour incubation. Cells were treated with vehicle or AF38469 (40 nM, 400 nM) for three hours then immunocytochemistry was performed with anti-GFP (Abcam, ab13970) and 4′, -diamidino-2-phenylindole-dihydrochloride (DAPI). Cells were imaged on the Nikon AIR confocal microscope. For (C), wild type N2A cells were plated and transfected with a pEGFP-N1-TFEB plasmid for a 24-hour incubation. Cells were dyed and imaged on the CellInsight™ CX7 High-Content Screening Platform (CX7) then treated with media containing vehicle or AF38469 (40 nM, 400 nM). Cells were imaged every 30 minutes for three hours on the CX7 with incubation between imaging sessions, and results were quantified. The statistics were analyzed using Graphpad Prism™. One-way ANOVA. Mean±S.E.M. Dunnett's multiple comparisons test compared to the vehicle treated group. *p<0.05,**p<0.01, ***p<0.001, ****p<0.0001. These data showed that treatment with AF38469 stimulated the nuclear translocation (i.e., activation) of TFEB. TFEB is a transcription factor that stimulates transcription of genes that are necessary for lysosomal biogenesis and function. Thus, increased TFEB nuclear translocation is expected to stimulate lysosomal biogenesis and function.

Treatment with AF38469 stimulates TFE3 nuclear translocation as monitored via time course study. See FIG. 4 for data. For (A) and (B), wild type N2A cells were plated and transfected with a pEGFP-N1-TFE3 plasmid for a 24-hour incubation. Cells were treated with vehicle or AF38469 (40 nM, 400 nM) for three hours then immunocytochemistry was performed with anti-GFP (Abcam, ab13970) and 4′, -diamidino-2-phenylindole-dihydrochloride (DAPI). Cells were imaged on the Nikon AIR confocal microscope. For (C), wild type N2A cells were plated and transfected with a pEGFP-N1-TFE3 plasmid for a 24-hour incubation. Cells were dyed and imaged on the CellInsight™ CX7 High-Content Screening Platform (CX7) then treated with media containing vehicle or AF38469 (40 nM, 400 nM). Cells were imaged every 30 minutes for three hours on the CX7 with incubation between imaging sessions, and results were quantified. The statistics were analyzed using Graphpad Prism™. One-way ANOVA. Mean±S.E.M. Dunnett's multiple comparisons test compared to the vehicle treated group. *p<0.05, **p<0.01, ***p<0.001, ****p<0.0001. This data indicates that treatment with AF38469 stimulates the nuclear translocation (i.e., activation) of TFE3. TFEB is a transcription factor that stimulates transcription of genes that are necessary for lysosomal biogenesis and function. Thus, increased TFE3 nuclear translocation is expected to stimulate lysosomal biogenesis and function.

Comparative transcriptomic analysis of differentially expressed genes in AF38469 treated and vehicle treated wild type mouse embryonic fibroblasts. See FIG. 5 for data. Wild type mouse embryonic fibroblasts were treated with vehicle or AF38469 (40 nM). Cells were plated on in vitro day 0, and cells were dosed with drug-containing media on DIV3, collection and RNA isolation occurred on DIV5. RNA sequencing and differentially expressed gene (DEG) analysis was performed with Novogene. DEGs were compared to lists of lysosomal genes and genes under TFEB regulation. These data indicate that AF38469 stimulates the expression of TFEB target genes.

Treatment with AF38469 increases PPT1 and TPP1 enzyme activity in Batten disease mouse embryonic fibroblasts. See FIG. 6 for data. Since AF38469 increases the expression of lysosomal genes including the PPT1 and TPP1 enzymes, we asked whether AF38469 increases the overall activity levels of these enzymes in cellulo. Wild type (WT) and mutant cells were treated with vehicle or AF38469 (40 nM). Cells were plated on day 0 and dosed with drug-containing media on DIV3 and DIV5. Cells were lysed, and protein quantification and enzyme activity assay were performed on DIV7. Data was analyzed with GraphPad Prism™. Two-way ANOVA. Mean±S.E.M. Tukey's multiple comparisons test. *p<0.05, **p<0.01, ***p<0.001, ****p<0.0001. Palmitoyl-protein thioesterase 1 (PPT1) is the lysosomal enzyme mutated in CLN1 disease and removes thioester-linked fatty acyl groups from modified cysteines of substrate proteins. Tripeptidyl peptidase 1 (TPP1) is the lysosomal enzyme mutated in CLN2 disease and has broad substrate specificity against various proteins degraded in the lysosome after cleavage. These data indicate that treatment with AF38469 increases the function of the lysosome in cellulo, likely through increased production of lysosomal proteins.

Since AF38469 reduced lysosomal pathology and increased lysosomal function in cell models of diverse lysosomal storage disorders, we asked whether treatment with AF38469 could reduce histopathological markers of disease severity in animal models of CLN2- and CLN3-Batten disease. We performed a series of experiments in which Cln2^{R207X} and Cln3^{Δex78} mouse models were treated with varying doses of AF38469 in drinking water. We first treated mice with 3.125 μg/ml and 78.125 μg/ml doses, mirroring those used for strong sortilin inhibition in other studies for unrelated disorders. Results showed that the lowest dose exerted the greatest benefit, leading us to enroll an additional study with lower doses of 0.03125 μg/ml or 0.3125 μg/ml. These lower doses exerted profound benefits across multiple histopathological and behavioral outcomes (FIGS. 7-12), demonstrating that sortilin inhibition, such as at low doses relative to standard dosing, is a useful treatment strategy for lysosomal storage disorders.

Short-term chronic treatment with AF38469 reduces storage material burden in Cln2^{R207X} and Cln3^{Δex7/8} mice. See FIG. 7 for data. Beginning at wean, wild type and Cln2^{R207X} mice were treated with vehicle (0.2408% DMSO) or AF38469 (0.03125 μg/ml, 0.3125 μg/ml, 3.125 μg/ml, 78.125 μg/ml) continuously through the drinking water continuing until sacrifice at 11 weeks. Beginning at wean, wild type and Cln3^{Δex7/8} mice were treated with vehicle (0.2408% DMSO) or AF38469 (0.03125 μg/ml, 0.3125 μg/ml, 3.125 μg/ml, 78.125 μg/ml) continuously through the drinking water until sacrifice at 16 weeks. Upon sacrifice, one hemisphere of brain was sectioned at 50 microns on a vibratome; DAB staining immunohistochemistry was then performed using anti-SubC (Abcam, ab181243) anti-rabbit biotinylated (Vector Labs, BA-1000). Sections were imaged and analyzed using an Aperio Digital Pathology Slide Scanner (AT2) and associated software. Images were extracted from S1BF of the somatosensory cortex and the VPM/VPL of the thalamus, key brain regions for Batten disease pathology. Percent area of immunoreactivity were quantified using a threshold analysis in ImageJ™. Statistical analyses were performed using GraphPad Prism™ (v8.4.3; San Diego, CA). The y-axis of the bar graphs shows “% Area” units which indicate % of the total cell area. Increasing concentrations of AF38469 and corresponding genotypes are shown along the x-axis. Nested one-way ANOVA. Mean±S.E.M. Dunnett's multiple comparisons test compared to the mutant vehicle treated group. *p<0.05, **p<0.01, ***p<0.001, ****p<0.0001. SubC is a major constituent of the autofluorescent storage material in NCLs. These data indicate that short term treatment with AF8469 reduces SubC accumulation in Cln2^{R207X} and Cln3^{Δex7/8} and has an inverse drug effect in which lower concentrations of the drug improved the lysosomal storage material burden of mitochondrial ATP synthase subunit C more than the higher doses. There appeared to be a greater positive effect on pathology in Cln3^{Δex7/8} mice compared to the Cln2^{R207X} mice, likely due to the disease progression differences between these two models (Cln2^{R207X} mice exhibit earlier and more rapid disease progression, with substantial pathology already present prior to initiation of treatment at weaning).

Short-term chronic treatment with AF38469 impacts glial activation in Cln2^{R207X} mice. See FIG. 8 for data. Beginning at wean, wild type and Cln2^{R207X} mice were treated with vehicle (0.2408% DMSO) or AF38469 (0.03125 μg/ml, 0.3125 μg/ml, 3.125 μg/ml, 78.125 μg/ml) continuously through the drinking water continuing until sacrifice at 11 weeks. Upon sacrifice, one hemisphere of brain was sectioned at 50 microns on a vibratome; DAB staining immunohistochemistry was then performed using anti-GFAP (Dako, Z0334; 1:5000), anti-CD68 (AbD Serotec, MCA1957; 1:2000), anti-rabbit biotinylated (Vector Labs, BA-1000 1:1000), and anti-rat (Vector Labs, BA-9400 1:1000). Sections were imaged and analyzed using an Aperio Digital Pathology Slide Scanner (AT2) and associated software. Images were extracted from S1BF of the somatosensory cortex and the VPM/VPL of the thalamus. Percent area of immunoreactivity were quantified using a threshold analysis in ImageJ™. Statistical analyses were performed using GraphPad Prism™ (v8.4.3; San Diego, CA). The y-axis of the bar graphs shows “% Area” units which indicate % of the total cell area. Increasing concentrations of AF38469 and corresponding genotypes are shown along the x-axis. Nested one-way ANOVA. Mean±S.E.M. Dunnett's multiple comparisons test compared to the mutant vehicle treated group. *p<0.05, **p<0.01, ***p<0.001, ****p<0.0001. Reactive gliosis including activated microglia and reactive astrocytes is one of the histopathologies associated with neurodegenerative diseases such as NCLs. Short-term treatment with AF38469 in Cln2^{R207X} mice at higher doses increased or had no impact on CD68 and GFAP reactivity. Short-term treatment with AF38469 in Cln2^{R207X} mice at lower doses significantly decreased CD68 reactivity and had no significant impact on GFAP reactivity although there is a trend towards a decrease.

Short-term chronic treatment with AF38469 impacts glial activation in Cln3^{Δex7/8} mice. See FIG. 9 for data. Beginning at wean, wild type and Cln3^{Δex7/8} mice were treated with vehicle (0.2408% DMSO) or AF38469 (0.03125 μg/ml, 0.3125 μg/ml, 3.125 μg/ml, 78.125 μg/ml) continuously through the drinking water continuing until sacrifice at 16 weeks. Upon sacrifice, one hemisphere of brain was sectioned at 50 microns on a vibratome; DAB staining immunohistochemistry was then performed using anti-GFAP (Dako, Z0334; 1:5000), anti-CD68 (AbD Serotec, MCA1957; 1:2000), anti-rabbit biotinylated (Vector Labs, BA-1000 1:1000), and anti-rat (Vector Labs, BA-9400 1:1000). Sections were imaged and analyzed using an Aperio Digital Pathology Slide Scanner (AT2) and associated software. Images were extracted from S1BF of the somatosensory cortex and the VPM/VPL of the thalamus. Percent area of immunoreactivity were quantified using a threshold analysis in ImageJ™. Statistical analyses were performed using GraphPad Prism™ (v8.4.3; San Diego, CA). The y-axis of the bar graphs shows “% Area” units which indicate % of the total cell area. Increasing concentrations of AF38469 and corresponding genotypes are shown along the x-axis. Nested one-way ANOVA. Mean±S.E.M. Dunnett's multiple comparisons test compared to the mutant vehicle treated group. *p<0.05, **p<0.01, ***p<0.001, ****p<0.0001. Reactive gliosis including activated microglia and reactive astrocytes is one of the histological pathologies associated with neurodegenerative diseases such as NCLs. Short-term treatment with AF38469 in Cln3^{Δex7/8} mice at higher doses increased or had no impact on CD68 and GFAP reactivity. Short-term treatment with AF38469 in Cln3^{Δex7/8} mice at lower doses significantly decreased CD68 and GFAP reactivity in somatosensory cortex and the thalamus.

Short-term chronic treatment with AF38469 reduced tremor phenotype in Cln2^{R207X} mice. See FIG. 10 for data. Beginning at wean, wild type and Cln2^{R207X} mice were treated with vehicle (0.2408% DMSO) or AF38469 (0.03125 μg/ml, 0.3125 μg/ml, 3.125 μg/ml, 78.125 μg/ml) continuously through the drinking water. At 10 weeks of age, wild type and Cln2^{R207X} mice were evaluated on the force plate actimeter. Evaluation of tremors with the force plate actimeter software produces a tremor index score based on the tremor frequency 5-10 Hz (A), 10-15 Hz (B), 15-20 Hz (C) and 20-25 Hz (D). Data was analyzed with Graphpad. Two-way ANOVA. Mean±S.E.M. Dunnett's multiple comparisons test. *p<0.05, **p<0.01, ***p<0.001, ****p<0.0001. Cln2^{R207X} mice show a tremor phenotype similar to one observed in patients. This data showed that short-term treatment with lower concentrations of AF38469 was able to significantly reduce tremor index scores observed in Cln2^{R207X} mice.

Short-term chronic treatment with AF38469 does not impact body weight of wild type mice but rescues body weight of Cln2^{R207X} mice. See FIG. 11 for data. Beginning at wean, wildtype and Cln2^{R207X} mice were treated with vehicle (0.2408% DMSO) or AF38469 (0.03125 μg/ml, 0.3125 μg/ml, 3.125 μg/ml, 78.125 μg/ml) continuously through the drinking water until sacrifice at 11 weeks age. Body weight was measured bi-weekly. The data was analyzed with Graphpad. Two-way ANOVA. Mean S.E.M. Dunnett's multiple comparisons test. *p<0.05, **p<0.01, ***p<0.001, ****p<0.0001. This data showed that short-term treatment with AF38469 does not impact body weights of wild type mice, but restores healthy body weights of 10 month old Cln2^{R207X} mice.

Short-term chronic treatment with AF38469 does not impact body weight of wild type or Cln3^{Δex7/8} mice. See FIG. 12 for data. Beginning at wean, wild type and Cln3^{Δex7/8} mice were treated with vehicle (0.2408% DMSO) or AF38469 (0.03125 μg/ml, 0.3125 μg/ml, 3.125 μg/ml, 78.125 μg/ml) continuously through the drinking water until sacrifice at 16 weeks age. Body weight was measured monthly. The data was analyzed with Graphpad. Two-way ANOVA. Mean±S.E.M. Dunnett's multiple comparisons test. *p<0.05, **p<0.01, ***p<0.001, ****p<0.0001. This data showed that short-term treatment with AF38469 does not impact body weights of wild type and Cln3^{Δex7/8} mice.

### Conclusion

To test whether sortilin-mediated trafficking of lysosomal machinery still functions at any level in the absence of CLN3, we applied AF38469, a potent sortilin inhibitor, to WT and CLN3-deficient cells. Since AF38469 would be expected to block trafficking of lysosomal cargoes, we expected to observe increased lysosomal storage substrates in treated WT cells. If AF38469 had a similar effect in CLN3-deficient cells, this would suggest that sortilin-mediated trafficking is still operating to an appreciable extent in this disease state. If AF38469 had no effect, this would suggest that sortilin-mediated trafficking is already compromised to a profound extent. Surprisingly, we found that sortilin inhibition strongly reduced the accumulation of lysosomal autofluorescent storage material (ASM) in CLN3-deficient cell models (FIG. 1B; FIG. 2B). We also screened the compound in other cell models of lysosomal storage disorders and observed similar benefits. These results demonstrated an unexpected and potent result of sortilin-inhibition in lysosomal storage disorders—decreased accumulation of lysosomal storage substrates. This benefit led to reductions in other downstream components of pathology (e.g. neuroinflammation, tremor index scores) in mouse models of lysosomal storage disorders. The dose-response relationship suggested that lower doses actually have more potent effects in several models, which suggests that the observed benefits may be the result of a hormetic response wherein mild sortilin inhibition stimulates compensatory responses in the cell that benefit lysosomal function.

### REFERENCES

1. Brozzi, A., Urbanelli, L., Luc Germain, P., Magini, A., & Emiliani, C. (2013). hLGDB: a database of human lysosomal genes and their regulation. Database, 2013.
2. Brudvig, J. J.; Weimer, J. M., On the cusp of cures: Breakthroughs in Batten disease research. Current opinion in neurobiology 2022, 72, 48-54.
3. Cotman S L, Vrbanac V, Lebel L A, Lee R L, Johnson K A, Donahue L R, Teed A M, Antonellis K, Bronson R T, Lerner T J, MacDonald M E. Cln3(Deltaex7/8) knock-in mice with the common JNCL mutation exhibit progressive neurologic disease that begins before birth. Hum Mol Genet. 2002 Oct. 15; 11(22):2709-21. doi: 10.03125093/hmg/11.22.2709. PMID: 12374761.
4. Gao, H, Boustany, R M, Espinola, J A. Cotman, S L, Srinidhi, L, Antonellis, K A, Gillis, T, Qin, X, Liu, S, Donahue, L R, et al. (2002). Mutations in a novel CLN6-encoded transmembrane protein cause variant neuronal ceroid lipofuscinosis in man and mouse. Am J Hum Genet 70: 324-335.
5. Geraets, R. D.; Langin, L. M.; Cain, J. T.; Parker, C. M.; Beraldi, R.; Kovacs, A. D.; Weimer, J. M.; Pearce, D. A., A tailored mouse model of CLN2 disease: A nonsense mutant for testing personalized therapies. PLoS One 2017, 12 (5), e0176526.
6. Johnson, T. B.; Cain, J. T.; White, K. A.; Ramirez-Montealegre, D.; Pearce, D. A.; Weimer, J. M., Therapeutic landscape for Batten disease: current treatments and future prospects. Nat Rev Neurol 2019, 15 (3), 161-178.
7. Kousi, M., Lehesjoki, A. E., & Mole, S. E. (2012). Update of the mutation spectrum and clinical correlations of over 360 mutations in eight genes that underlie the neuronal ceroid lipofuscinoses. Human mutation, 33(1), 42-63.
8. Metcalf, D. J., Calvi, A. A., Seaman, M., Mitchison, H. M., & Cutler, D. F. (2008). Loss of the Batten disease gene CLN3 prevents exit from the TGN of the mannose 6-phosphate receptor. Traffic (Copenhagen, Denmark), 9(11), 1905-1914. https://doi.org/10.0312511l/j.1600-0854.2008.00807.x
9. Miller, J. N., Kovics, A. D., & Pearce, D. A. (2015). The novel Cln1(R151X) mouse model of infantile neuronal ceroid lipofuscinosis (INCL) for testing nonsense suppression therapy. Human molecular genetics, 24(1), 185-196. https://doi.org/10.03125093/hmg/ddu428
10. Mole, S. E.; Cotman, S. L., Genetics of the neuronal ceroid lipofuscinoses (Batten disease). Biochim Biophys Acta 2015, 1852 (10 Pt B), 2237-41.
11. Palmer, D. N.; Feamley, I. M.; Walker, J. E.; Hall, N. A.; Lake, B. D.; Wolfe, L. S.; Haltia, M.; Martinus, R. D.; Jolly, R. D., Mitochondrial ATP synthase subunit c storage in the ceroid-lipofuscinoses (Batten disease). Am J Med Genet 1992, 42 (4), 561-7.
12. Ranta, S., Zhang, Y., Ross, B., Lonka, L., Takkunen, E., Messer, A., . . . & Lehesjoki, A. E. (1999). The neuronal ceroid lipofuscinoses in human EPMR and mnd mutant mice are associated with mutations in CLN8. Nature genetics, 23(2), 233-236.
13. Rhost, S., Hughes, F., Harrison, H. et al. Sortilin inhibition limits secretion-induced progranulin-dependent breast cancer progression and cancer stem cell expansion. Breast Cancer Res 20, 137 (2018). https://doi.org/10.03125186/s13058-018-1060-5
14. Rouillard A D, Gundersen G W, Fernandez N F, Wang Z, Monteiro C D, McDermott M G, Ma'ayan A. The harmonizome: a collection of processed datasets gathered to serve and mine knowledge about genes and proteins. Database (Oxford). 2016 Jul. 3; 2016. pii: baw100.
15. Saito, T., & Nakatsuji, N. (2001). Efficient gene transfer into the embryonic mouse brain using in vivo electroporation. Developmental biology, 240(1), 237-246.
16. Santavuori, P., Neuronal ceroid-lipofuscinoses in childhood. Brain Dev 1988, 10 (2), 80-3.
17. Schroder, T. J.; Christensen, S.; Lindberg, S.; Langgard, M.; David, L.; Maltas, P. J.; Eskildsen, J.; Jacobsen, J.; Tagmose, L.; Simonsen, K. B., The identification of AF38469: an orally bioavailable inhibitor of the VPS10P family sorting receptor Sortilin. Bioorganic & medicinal chemistry letters 2014, 24 (1), 177-180.
18. Yasa, S., Modica, G., Sauvageau, E., Kaleem, A., Hermey, G., & Lefrancois, S. (2020). CLN3 regulates endosomal function by modulating Rab7A-effector interactions. Journal of cell science, 133(6), jcs234047.
19. Richner, M., et al., Sortilin gates neurotensin and BDNF signaling to control peripheral neuropathic pain. Sci Adv, 2019. 5(6): p. eaav994.
20. Teng, H. K., et al., ProBDNF induces neuronal apoptosis via activation of a receptor complex of p75NTR and sortilin. J Neurosci, 2005. 25(22): p. 5455-63.
21. Canuel, M., Y. Libin, and C. R. Morales, The interactomics of sortilin: an ancient lysosomal receptor evolving new functions. Histol Histopathol, 2009. 24(4): p. 481-92.
22. Pallesen, L. T. and C. B. Vaegter, Sortilin and SorLA regulate neuronal sorting of trophic and dementia-linked proteins. Mol Neurobiol, 2012. 45(2): p. 379-87.
23. Canuel, M., et al., AP-1 and retromer play opposite roles in the trafficking of sortilin between the Golgi apparatus and the lysosomes. Biochem Biophys Res Commun, 2008. 366(3): p. 724-30.
24. Stachel, S. J., et al., Identification of potent inhibitors of the sortilin-progranulin interaction. Bioorg Med Chem Lett, 2020. 30(17): p. 127403

### Example 3

### Summary

Batten disease (neuronal ceroid lipofuscinosis; NCL) is a family of rare, fatal, neuropediatric lysosomal storage disorders, typically presenting in early childhood with memory/learning decline, blindness, and loss of motor function. Each of these disorders is caused by mutations in 1 of 13 different genes which encode a variety of soluble and transmembrane NCL proteins. LM11A-31 is a small molecule p75^{NTR} ligand. We tested the effectiveness of LM11A-31 in reducing Batten's disease pathology in vitro and in the Cln3^{Δex7/8} mouse model. LM11A-31 showed promising results at decreasing autofluorescent storage material and signal in various NCLs in neurons and MEFs in vitro, indicating LM11A-31 improves lysosome health. A one-dose efficacy study was executed to study therapeutic efficacy of LM11A-31 using a Cln3^{Δex7/8} mouse model. A decrease of SubC was seen in the VPM/VPL region of the thalamus, however an increase was seen in the S1BF region of the somatosensory cortex. There was a decreasing trend of GFAP in the VPM/VPL and S1BF, which could indicate a decrease in astrocytosis.

### Study Groups

In vitro groups consisted of 3 cell culture wells per treatment per genotype. Mouse embryonic fibroblasts (MEFs) and primary cortical neurons from wild type and 5 mutant lines were utilized, (WT, Cln1^{R151X}, Cln2^{R207X}, Cln3^{Δex7/8}, Cln6^{nclf}, Cln8^{mnd}). Each group consisted of 8 mice comprised of mixed sexes, age-matched across all groups (N=4/sex/group). Two mouse strains were utilized, C57BL/6J Wild-Type (WT) and a knock-in homozygous Cln3 mutant (Cln3^{Δex7/8}) Total study N=32.

### Methods

Cells were dosed with LM11A-31 on DIV3 and DIV5; on DIV7, cells were collected and analyzed. Cln3^{Δex7/8} homozygous knock-in mice and C57/6J controls were dosed with either vehicle or LM11A-31 via drinking water starting at wean (P21) with continued dosing through collection at 16 weeks. Water was replaced weekly. Animals were collected at 16 weeks of age.

### Test Article(s)

In in vitro models, LM11A-31 (VulcanChem, catalog #VC281127) was tested in concentrations of 100 nM, 1 μM, and 10 μM in 1% DMSO. Vehicle treated cells were dosed with 1% DMSO vehicle. In in vivo models, LM11A-31 (VulcanChem, catalog #VC281127) was tested in concentrations of 0.6 mg/mL or 1.92 mg/mouse/day (targeted dose of ˜75 mg/kg) dosed in drinking water. Vehicle treated animals were dosed with drinking water containing DI water. LM11A-31 and vehicle water was prepared weekly.

### Test System

All animals are maintained on a C57BL/6J background and housed under identical conditions. Wild type animals lacked these mutations. All animal studies were conducted in an AAALAC accredited facility under NIH guidelines and approved by Sanford IACUC (USDA license 46-R-0009). Cln1^{R151X} point mutant mice (The Jackson Laboratory #026197) replicate a nonsense mutation commonly seen in CLN1 patients. Cln2^{R207X} point mutant mice (The Jackson Laboratory 4030696) replicate a nonsense mutation seen in CLN2 patients. Cln3^{Δex7/8} knock-in mutant mice (The Jackson Laboratory 4017895) replicate a common mutation seen in CLN3 patients in which deletions of exons 7 and 8 lead to a premature stop codon. Cln6^{nclf} mice (The Jackson Laboratory #003605) replicate a common mutation seen in CLN6 patients, in which an insertion leads to a premature stop codon. Cln8^{mnd} mutant mice (The Jackson Laboratory #001612) replicate a spontaneous mutation commonly found in CLN8 patients. C57BL/6J (Wild type (WT)) animals lacked these mutations. Mouse embryonic fibroblasts (MEFs) and primary cortical neurons (PNCs) were collected from WT or mutant mice on embryonic day 15.5, and primary cell cultures are generated for in vitro testing. Cln3^{Δex7/8} mice were dosed through drinking water starting at wean.

### Experimental Procedures

### Study Groups

In vitro studies:
   WT MEFs in triplicate treated with vehicle, 100 nM LM11A-31, 1 nM LM11A-31, 10 μM LM11A-31 for LysoTracker™ and ASM analyses.
   Cln1^{R151X} MEFs in triplicate treated with vehicle, 100 nM LM11A-31, 1 μM LM11A-31, 10 μM LM11A-31 for LysoTracker™ and ASM analyses.
   Cln2^{R207X} MEFs in triplicate treated with vehicle, 100 nM LM11A-31, 1 μM LM11A-31, 10 μM LM11A-31 for LysoTracker™ and ASM analyses.
   Cln3^{Δex7/8} MEFs in triplicate treated with vehicle, 100 nM LM11A-31, 1 μM LM11A-31, 10 μM LM11A-31 for LysoTracker™ and ASM analyses.
   Cln6^{nclf} MEFs in triplicate treated with vehicle, 100 nM LM11A-31, 1 μM LM11A-31, 10 μM LM11A-31 for LysoTracker™ and ASM analyses.
   Cln8^{mnd} MEFs in triplicate treated with vehicle, 100 nM LM11A-31, 1 μM LM11A-31, 10 μM LM11A-31 for LysoTracker™ and ASM analyses.
   WT PNCs in triplicate treated with vehicle, 100 nM LM11A-31, 1 μM LM11A-31, 10 μM LM11A-31 for LysoTracker™ and ASM analyses.
   Cln1^{R151X} PNCs in triplicate treated with vehicle, 100 nM LM11A-31, 1 μM LM11A-31, 10 μM LM11A-31 for LysoTracker™ and ASM analyses.
   Cln2^{R07X} PNCs in triplicate treated with vehicle, 100 nM LM11A-31, 1 μM LM11A-31, 10 μM LM11A-31 for LysoTracker™ and ASM analyses.
   Cln3^{Δex7/8} PNCs in triplicate treated with vehicle, 100 nM LM11A-31, 1 μM LM11A-31, 10 μM LM11A-31 for LysoTracker™ and ASM analyses.
   Cln6^{nclf} PNCs in triplicate treated with vehicle, 100 nM LM11A-31, 1 μM LM11A-31, 10 μM LM11A-31 for LysoTracker™, and ASM analyses.
   Cln8^{mnd} PNCs in triplicate treated with vehicle, 100 nM LM11A-31, 1 μM LM11A-31, 10 μM LM11A-31 for LysoTracker™ and ASM analyses.
In vivo dose study in Cln3^{Δex7/8} mice
   8 (4 per sex) WT mice were treated with vehicle (water) from wean until 16 weeks of age as a control for the Cln3^{Δex7/8} group
   8 (4 per sex) WT mice were treated with 0.6 mg/mL LM11A-31 from wean until 16 weeks of age as a control for the Cln3^{Δex7/8} group
   8 (4 per sex) Cln3^{Δex7/8} mice were treated with vehicle (water) from wean until 16 weeks of age
   8 (4 per sex) Cln3^{Δex7/8} mice were treated with 0.6 mg/mL LM11A-31 from wean until 16 weeks of age

### Experimental Details

Specific experimental details are included in figure legends (FIGS. 13-17). In general, after collection and preparation, MEFs and PNCs were dosed with vehicle or LM1TA-31 (100 nM, 1 μM, or 10 μM) on DIV3 and DIV5. On DIV7, cells were treated with the following dyes: Hoechst 33342 (1:1000 dilution), LysoTracker™ Red DND-99 (1:10,000 dilution), fixed, and imaged. Mice were given vehicle or LM11A-31 (0.6 mg/mL or 1.92 mg/mouse/day, targeted dose of ˜75 mg/kg, respectively, given a mouse's daily water intake of approximately 3.2 ml per day) in drinking water beginning at wean. At 16 weeks of age, Cln3^{Δex7/8} mice along with age matched wild type control mice were CO₂ euthanized and intracardially perfused with PBS. Brains were collected and placed on a 1 mm sagittal brain block. Brains were sliced at the midline and 3 mm right of the midline. One hemisphere was fixed in 4% PFA and sectioned at 50 microns on a vibratome. Immunohistochemistry was performed on free-floating sections. For mitochondrial ATP synthase subunit C (SubC), glial fibrillary acidic protein (GFAP), and cluster of differentiation 68 (CD68) immunolabeling, primary antibodies included anti-SubC (Abcam, ab181243; 1:1000), anti-GFAP (Dako, Z0334; 1:5000), anti-CD68 (AbD Serotec, MCA1957; 1:2000), and secondary antibodies included anti-rabbit biotinylated (Vector Labs, BA-1000 1:1000) and anti-rat (Vector Labs, BA-9400 1:1000). Coverslips were mounted on slides using antifade-mounting media and were stored in the dark before imaging. Sections were imaged and analyzed using an Aperio Digital Pathology Slide Scanner (AT2) and associated software. Images were extracted from S1BF of the somatosensory cortex and the VPM/VPL of the thalamus, with multiple images taken of multiple tissues from each animal.

### Data Analysis

Percent area of fluorescent signal and immunoreactivity were quantified using a threshold analysis in ImageJ™. Statistical analyses were performed using GraphPad Prism™ (v8.4.3; San Diego, CA). Details of the specific tests are noted in the figure legends. In general, for in vitro analysis, two-way ANOVA was employed with Scheffe post hoc for multiple comparisons. In general, for in vivo analysis, one-way nested ANOVA was employed with Sidak's post hoc for multiple comparisons. and outliers were removed with the ROUT method, Q=1%. *p<0.05, **p<0.01, ***p<0.001, ****p<0.0001

### Interpretation of Results

An increase in percent area or total area of positive autofluorescent storage material (ASM) signal in untreated NCL MEFs or PNCs relative to age-matched, wild type MEFs or PNCs is considered an increase in the accumulation of autofluorescent storage material, reflecting disease-related lysosomal dysfunction.
A decrease in percent area or total area of positive autofluorescent storage material signal in LM11A-31 treated MEFs and PNCs relative to age-matched, vehicle treated MEFs and PNCs is considered a reduction in the accumulation of autofluorescent storage material and thus a reduction in disease-related lysosomal dysfunction.
Lysotracker™ is an acidophilic dye that labels lysosomes and accumulation increases with an increase in lysosome size or number. Cell and mouse models of lysosomal storage disorders often have increased accumulation of lysosomes and therefore have a stronger Lysotracker™ signal. Drug-related changes in Lysotracker™ signal could be due to reduction of lysosomal pathology, changes in lysosomal biogenesis, or both, and are thus best interpreted alongside other measures of lysosomal function such as autofluorescent storage material.
An increase in percent area or total area of positive SubC in vehicle treated Batten disease mice relative to age-matched vehicle treated wild type mice is considered an increase in the accumulation of mitochondrial ATP Synthase Subunit C, reflecting disease-related lysosomal dysfunction.
A decrease in Percent area or Total area of positive SubC in LM1TA-31 treated mice relative to vehicle treated Batten disease mice is considered a reduction in the accumulation of mitochondrial ATP Synthase Subunit C and thus a reduction in disease-related lysosomal dysfunction.
An increase in Percent area or Total area of positive CD68 staining in vehicle treated Batten disease mice relative to age-matched vehicle treated wild type mice is considered an increase microgliosis and neuroinflammation.
A decrease in Percent area or Total area of positive CD68 staining in LM11A-31 treated mice relative to vehicle treated Batten disease mice is considered a reduction in the accumulation of microgliosis.
An increase in Percent area or Total area of positive GFAP staining in vehicle treated Batten disease mice relative to age-matched vehicle treated wild type mice is considered an increase in the accumulation of astrogliosis and neuroinflammation.
A decrease in Percent area or Total area of positive GFAP staining in LM11A-31 treated mice relative to vehicle treated Batten disease mice is considered a reduction in the accumulation of astrogliosis.

### Results

Used in vitro and in vivo to treat Batten's disease, LM11A-31 was found to have the following effects.

Wild type (WT) and mutant cells were treated with vehicle or LM11A-31 (100 nM, 1 μM, 10 μM). Cells were plated on day 0 and dosed with drug-containing media on DIV3 and DIV5. Analysis occurred on DIV7, and data is shown in FIG. 13. Concentrations of drug are shown on the x-axis above each image column; genotypes are shown to the left of each row. The v-axis of the bar graphs shows “Total Area” units which indicate % of the total cell area. Increasing concentrations of LM11A-31 are shown along the x-axis. Two-way ANOVA with a Scheffe post-hoc test *=significantly (p<0.05) different from wild type (WT) vehicle. #=significantly (p<0.05) different from NCL vehicle. ASM trended towards reductions in Cln2^{R207X} and Cln3^{Δex7/8} in the mid to high doses in MEFs in vitro (FIG. 13A). Lysotracker™ signal was significantly reduced in Cln1^{R151X} and Cln2^{R207X} in the mid dose, with a decreasing trend seen in varying doses in Cln3^{Δex7/8} and Cln6^{nclf} (FIG. 13B). ASM was significantly reduced in neurons in varying doses in all tested NCLs in vitro (FIG. 14A). Lysotracker™ signal was significantly reduced in Cln2^{R207X} and Cln6^{nclf} neurons compared to vehicle controls (FIG. 14B).

Since LM11A-31 improves lysosomal health and function across diverse cell models of lysosomal storage disorders, we asked whether the drug could be activating transcriptional networks that regulate lysosomal function. The transcription factors TFEB and TFE3 are master regulators of lysosomal biogenesis and function. We hypothesized that LM11A-31 could exert benefits by modulating the nuclear translocation of TFEB and/or TFE3. We tested this in an in vitro system in which GFP fusions of TFEB or TFE3 are expressed and monitored with live imaging (FIG. 15A). After 90 minutes of incubation with 100 nM of LM11A-31, there was a significant increase in TFEB nuclear translocation. After 90 minutes of incubation with 100 nM and 1 μM of LM11A-31, there was a significant increase in TFE3 nuclear translocation. n=5000-6500 cells/treatment. These data showed that treatment with LM11A-31 stimulated the nuclear translocation (i.e., activation) of TFEB and TFE3.

Since LM11A-31 stimulates the nuclear translocation of transcription factors responsible for lysosome biogenesis, we asked whether LM11A-31 increases the overall activity levels of lysosomalenzymes in cellulo. We found that LM11A-31 activity increased the overall levels of activity for PPT1 and Cathepsin D enzymes in cell lysates (FIG. 15B). Wild type (WT) and mutant cells were treated with vehicle or LM11A-31 (100 nM). Cells were plated on day 0 and dosed with drug-containing media on DIV3 and DIV5. Cells were lysed, and protein quantification and enzyme activity assay were performed on DIV7. Data was analyzed with GraphPad Prism™. Two-way ANOVA. Mean±S.E.M. Tukey's multiple comparisons test. *p<0.05, **p<0.01, ***p<0.001, ****p<0.0001. Treatment with LM11A-31 (100 nM) significantly increased PPT1 enzyme activity in wild type, Cln2^{R207X} and Cln3^{Δex7/8} MEFs when compared to the vehicle treated mutant control. n=5 wells/treatment. Treatment with LM11A-31 (100 nM) significantly increased TPP1 enzyme activity in wild type and Cln3^{Δex7/8} MEFs when compared to the vehicle treated mutant control. Treatment with LM11A-31 (100 nM) significantly decreased levels of Cathepsin D in Cln2^{R207X} and Cln3^{Δex7/8} MEFs. n=5 wells/treatment. Palmitoyl-protein thioesterase 1 (PPT1) is the lysosomal enzyme mutated in CLN1 disease and removes thioester-linked fatty acyl groups from modified cysteines of substrate proteins. Tripeptidyl peptidase 1 (TPP1) is the lysosomal enzyme mutated in CLN2 disease and has broad substrate specificity against various proteins degraded in the lysosome after cleavage. Mutations in the CTSD gene, encoding cathepsin d, a lysosomal protease that is involved in the recycling and degradation, is a known cause of Cln10 disease. These data indicate that treatment with LM11A-31 increases the function of the lysosome in cellulo, likely through increased production of lysosomal proteins.

Since LM11A-31 reduced lysosomal pathology and increased lysosomal function in cell models of diverse lysosomal storage disorders, we asked whether treatment with LM11A-31 could reduce histopathological markers of disease severity in animal models of CLN3-Batten disease. We performed a series of experiments in which the Cln3^{Δex7/8} mouse model was treated with 0.6 mg/mL of LM11A-31 in drinking water. See FIGS. 16 and FIG. 17. Beginning at wean, wild type and Cln3^{Δex7/8} mice were treated with vehicle (DI water) or LM11A-31 (0.6 mg/ml; 1.92 mg/mouse/day, targeted dose of ˜75 mg/kg) continuously through the drinking water continuing until sacrifice at 16 weeks. Upon sacrifice, one hemisphere of brain was sectioned at 50 microns on a vibratome; DAB staining immunohistochemistry was then performed using anti-GFAP (Dako, Z0334; 1:5000), anti-CD68 (AbD Serotec, MCA1957; 1:2000), anti-rabbit biotinylated (Vector Labs, BA-1000 1:1000), and anti-rat (Vector Labs, BA-9400 1:1000). Sections were imaged and analyzed using an Aperio Digital Pathology Slide Scanner (AT2) and associated software. The flash-frozen brain was sectioned into 16 μm slices and placed on slides. Slides were post-fixed for 20 minutes in chilled 10% neutral buffered formalin (NBF) and underwent serial ethanol dehydration. To quantify accumulation of autofluorescent storage material, DAPI was applied to slides and coverslipped with aqueous mounting media (Dako Faramount, Agilent, S302580-2). Sections were imaged using a Nikon NiE microscope and associated software. In Cln3^{Δex7/8} mice, significantly increased ASM levels were evident in the VPM/VPL of the thalamus and S1BF of the cortex, motor cortex (MC) and visual cortex (VC). Treatment with LM11A-31 reduced ASM to levels that were not statistically distinguishable from wild type in the VPM/VPL of the thalamus and in the VC (see FIG. 16). Similar trends were observed for SubC accumulation (see FIG. 17). Cln3^{Δex7/8} mice also exhibited elevations in GFAP and CD68 in both the cortex and thalamus. Treatment with LM11A-31 resulted in trends towards decreased levels of GFAP in the cortex and thalamus and CD68 in the thalamus (see FIG. 17).

### REFERENCES FOR EXAMPLE 3

1. Mole, S. E. and M. Gardiner, Molecular genetic analysis of neuronal ceroid lipofuscinosis. Int J Neurol, 1991. 25-26: p. 52-9.
2. Mole, S. E. and S. L. Cotman, Genetics of the neuronal ceroid lipofuscinoses (Batten disease). Biochim Biophys Acta, 2015. 1852(10 Pt B): p. 2237-41.
3. Simmons, D. A., et al., A small molecule p75NTR ligand, LM11A-31, reverses cholinergic neurite dystrophy in Alzheimer's disease mouse models with mid-to late-stage disease progression. PloS one, 2014. 9(8): p. e102136-e102136.
4. Simmons, D. A., et al., A small molecule p75NTR ligand normalizes signalling and reduces Huntington's disease phenotypes in R6/2 and BACHD mice. Hum Mol Genet, 2016. 25(22): p. 4920-4938.
5. Miller, J. N., A. D. Kovacs, and D. A. Pearce, The novel Cln1 (R151X) mouse model of infantile neuronal ceroid lipofuscinosis (INCL) for testing nonsense suppression therapy. Hum Mol Genet, 2015. 24(1): p. 185-96.
6. Mole, S. E. and S. L. Cotman, Genetics of the neuronal ceroid lipofuscinoses (Batten disease). Biochimica et Biophysica Acta (BBA)—Molecular Basis of Disease, 2015. 1852(10, Part B): p. 2237-2241.
7. Cotman, S. L., et al., Cln3(Deltaex7/8) knock-in mice with the common JNCL mutation exhibit progressive neurologic disease that begins before birth. Hum Mol Genet, 2002. 11(22): p. 2709-21.
8. Gao, H., et al., Mutations in a novel CLN6-encoded transmembrane protein cause variant neuronal ceroid lipofuscinosis in man and mouse. Am J Hum Genet, 2002. 70(2): p. 324-35.
9. Nguyen, T. V., et al., Small molecule p75NTR ligands reduce pathological phosphorylation and misfolding of tau, inflammatory changes, cholinergic degeneration, and cognitive deficits in AbetaPP(L/S) transgenic mice. J Alzheimers Dis, 2014. 42(2): p. 459-83.
10. Shi, J., F. M. Longo, and S. M. Massa, A small molecule p75NTR ligand protects neurogenesis after traumatic brain injury. STEM CELLS, 2013. 31(11): p. 2561-2574.
11. Palmer, D. N., et al., Mitochondrial ATP synthase subunit c storage in the ceroid-lipofuscinoses (Batten disease). Am J Med Genet, 1992. 42(4): p. 561-7.
12. Johnson, T. B., et al., Therapeutic landscape for Batten disease: current treatments and future prospects. Nat Rev Neurol, 2019. 15(3): p. 161-178.

### Example 4

### Summary

We characterized the use of semaglutide, a GLP1R agonist, in in vitro models of CLN1, CLN2, CLN3, CLN6, and CLN8, -Batten disease and in vivo in a mouse model of CLN3 Batten disease. Using the in vitro models, we showed that AF38469 reduced lysosomal pathology and enhanced lysosomal enzyme activity. Using the in vivo CLN3 Batten disease mouse model, we show that treatment with semaglutide reduced pathological hallmarks of disease. Collectively, our results demonstrate that semaglutide is a potent and effective modulator of lysosomal function that exhibits in vivo efficacy in a prototypical lysosomal storage disorder, suggesting that GLP1R agonism could have broad applicability as a therapeutic strategy for disorders characterized by lysosomal dysfunction.

### Study Groups

In vitro groups consisted of 3 wells per treatment per genotype. Mouse embryonic fibroblasts and primary cortical neurons from wild type and mutant mouse models were utilized (WT, Cln1^{R151X}, Cln2^{R207X}, Cln3^{Δex7/8}, Cln6^{nclf}, Cln8^{mnd}). In vivo groups consisted of 7-8 mice comprised of mixed and balanced sexes, age-matched across all groups. For the in vivo study, two mouse strains were utilized, C57BL/6J Wild-Type (WT) and Cln3^{Δex7/8} knock-in mutant mouse.

### Methods

Cells were dosed with semaglutide on DIV3 and DIV5; on DIV7. Beginning at wean, mice were dosed with vehicle or semaglutide (2.571 μg/mouse, 25.71 μg/mouse) via subcutaneous injection, three times a week, beginning at wean. Wildtype and Cln3^{Δex7/8} mice were sacrificed at 16 weeks of age.

In in vitro models, semaglutide (Creative Peptides, Catalog #910463-68-2) was tested in concentrations of 10 nM, 100 nM, and 1 μM in 1% DMSO. Vehicle treated cells were dosed with 1% DMSO. In in vivo models, semaglutide (Creative Peptides, Catalog #910463-68-2) was tested in concentrations of 2.571 μg/mouse and 25.71 μg/mouse prepared using the vehicle formulation for a total injection volume of 0.2 ml. Compound was stable in solution for 56 days at 4° C. Semaglutide was utilized at 98% HPLC purity.

TABLE 2
Semaglutide Formulation

| Ingredient | Amount | Unit |
|---|---|---|
| Semaglutide | 1.34 | mg |
| Disodium Phosphate | 1.42 | mg |
| Propylene Glycol | 14 | mg |
| Phenol | 5.5 | mg |
| H₂O | 1 | ml |

All animals were maintained on a C57BL/6J background and housed under identical conditions. All animal studies are conducted in an AAALAC accredited facility under NIH guidelines and approved by Sanford IACUC (USDA license 46-R-0009). Cln1^{R151X} point mutant mice (The Jackson Laboratory #026197) replicate a nonsense mutation commonly seen in CLN1 patients. (Miller, 2015) Cln2^{R207X} point mutant mice (The Jackson Laboratory #030696) replicate a nonsense mutation seen in CLN2 patients. (Mole, 2012) Cln3^{Δex7/8} knock-in mutant mice (The Jackson Laboratory #017895) replicate a common mutation seen in CLN3 patients in which deletions of exons 7 and 8 lead to a premature stop codon (Cotman, 2002). Cln6^{nclf} mice (The Jackson Laboratory #003605) replicate a common mutation seen in CLN6 patients, in which an insertion leads to a premature stop codon. (Gao. 2002) Cln8^{mnd} mutant mice (The Jackson Laboratory #001612) replicate a spontaneous mutation commonly found in CLN8 patients. (Ranta, 1999) C57BL/6J (Wildtype (WT)) mice lacked these mutations. Mouse embryonic fibroblasts (MEFs) and primary cortical neurons (PNCs) are collected from WT or NCL mice on embryonic day 15.5. Cln3^{Δex7/8} mice were dosed with vehicle or semaglutide three times a week through subcutaneous injections starting at wean.

### Study Groups

In vitro studies:
   WT MEFs in triplicate treated with vehicle, 10 nM semaglutide, 100 nM semaglutide, 1 μM semaglutide for LysoTracker™ and ASM analyses.
   Cln1^{R151X} MEFs in triplicate treated with vehicle, 10 nM semaglutide, 100 nM semaglutide, 1 μM semaglutide for LysoTracker™ and ASM analyses.
   Cln2^{R207X} MEFs in triplicate treated with vehicle, 10 nM semaglutide, 100 nM semaglutide, 1 μM semaglutide for LysoTracker™ and ASM analyses.
   Cln3^{Δex7/8} MEFs in triplicate treated with vehicle, 10 nM semaglutide, 100 nM semaglutide, 1 μM semaglutide for LysoTracker™ and ASM analyses.
   Cln6^{nclf} MEFs in triplicate treated with vehicle, 10 nM semaglutide, 100 nM semaglutide, 1 μM semaglutide for LysoTracker™ and ASM analyses.
   Cln8^{mnd} MEFs in triplicate treated with vehicle, 10 nM semaglutide, 100 nM semaglutide, 1 μM semaglutide for LysoTracker™ and ASM analyses.
   Cln11^{-/-} MEFs in triplicate treated with vehicle, 10 nM semaglutide, 100 nM semaglutide, 1 μM semaglutide for LysoTracker™ and ASM analyses.
   WT PNCs in triplicate treated with vehicle, 10 nM semaglutide, 100 nM semaglutide, 1 μM semaglutide for LysoTracker™ and ASM analyses.
   Cln1^{R151X} PNCs in triplicate treated with vehicle, 10 nM semaglutide, 100 nM semaglutide, 1 μM semaglutide for LysoTracker™ and ASM analyses.
   Cln2^{R207X} PNCs in triplicate treated with vehicle, 10 nM semaglutide, 100 nM semaglutide, 1 μM semaglutide for LysoTracker™ and ASM analyses.
   Cln3^{Δex7/8} PNCs in triplicate treated with vehicle, 10 nM semaglutide, 100 nM semaglutide, 1 μM semaglutide for LysoTracker™ and ASM analyses.
   Cln6^{nclf} PNCs in triplicate treated with vehicle, 10 nM semaglutide, 100 nM semaglutide, 1 μM semaglutide for LysoTracker™ and ASM analyses.
   Cln8^{mnd} PNCs in triplicate treated with vehicle, 10 nM semaglutide, 100 nM semaglutide, 1 μM semaglutide for LysoTracker™ and ASM analyses.
In vivo study (2.571 μg/mouse and 25.571 μg/mouse) in Cln3^{Δex7/8} mice
   8 (4 per sex) WT mice were treated with vehicle from wean until 16 weeks of age as a control for the Cln3^{Δex7/8} group
   8 (4 per sex) WT mice were treated with 2.571 μg/mouse semaglutide from until 16 weeks of age as a control for the Cln3^{Δex7/8} group
   8 (4 per sex) WT mice were injected three times per week with 25.71 μg/mouse semaglutide from wean until 16 weeks of age as a control for the Cln3^{Δex7/8} group
   8 (4 per sex) Cln3^{Δex7/8} were injected three times per week with vehicle from wean until 16 weeks of age
   8 (4 per sex) Cln3^{Δex7/8} were injected three times per week with 2.571 μg/mouse semaglutide from wean until 16 weeks of age
   8 (4 per sex) Cln3^{Δex7/8} were injected three times per week with 25.71 μg/mouse semaglutide from wean until 16 weeks of age

### Experimental Details

Specific experimental details are included in figure legends. In general, after collection and preparation, MEFs and PNCs were dosed with vehicle or semaglutide (10 nM, 100 nM. or 1 μM) on DIV3 and DIV5. On DIV7, cells were exposed to the following dyes: Hoechst 33342 (1:1000 dilution), LysoTracker™ Red DND-99 (1:10,000 dilution), fixed. and imaged. Mice were injected three times per week with vehicle or semaglutide (2.571 μg/mouse, 25.71 μg/mouse) beginning at wean. Body weight was measured at 4, 6, 8, 12, and 16 weeks of age. At 16 weeks of age, Cln3^{Δex7/8} mice along with age matched wildtype control mice were CO₂ euthanized and intracardially perfused with PBS. Brains were collected and placed on a 1 mm sagittal brain block. Brains were sliced at the midline and 3 mm right of the midline. The 3 mm sagittal piece was flash frozen with −50° C. isopentane and then sectioned on a cryostat at 16 μm and placed on slides. For ASM measurements, slides were counterstained with DAPI. The other hemisphere was fixed in 4% PFA and sectioned at 50 microns on a vibratome. Immunohistochemistry was performed on free-floating sections. For SubC, GFAP, and CD68 immunolabeling, primary antibodies included mitochondrial anti-ATP synthase subunit C (Abcam, ab181243; 1:1000), anti-GFAP (Dako, Z0334; 1:5000), anti-CD68 (AbD Serotec, MCA1957; 1:2000), and secondary antibodies included anti-rabbit biotinylated (Vector Labs, BA-1000 1:1000) and anti-rat (Vector Labs, BA-9400 1:1000). Coverslips were mounted on slides using antifade-mounting media and were stored in the dark before imaging. ASM slides were imaged on a Nikon ECLIPSE™ Ni-E upright microscope with a CoolSNAP™ DYNO camera. Images were extracted from S1BF of the somatosensory cortex, CA3 of the hippocampus, and the VPM/VPL of the thalamus, with multiple images taken of multiple tissues from each animal. IHC slides were imaged and analyzed using an Aperio Digital Pathology Slide Scanner (AT2) and associated software. Images were extracted from the S1BF of the somatosensory cortex, the CA3 of the hippocampus, and the VPM/VPL of the thalamus.

### Data Analysis

Percent area of fluorescent signal and immunoreactivity were quantified using a threshold analysis in ImageJ™. Statistical analyses were performed using GraphPad Prism™ (v8.4.3; San Diego, CA). Details of the specific tests are noted in the figure legends. In general, for in vitro analysis, two-way ANOVA was employed with Scheffe post hoc for multiple comparisons. In general, for in vivo analysis, one-way ANOVA was employed with Sidak's post hoc for multiple comparisons. and outliers were removed with the ROUT method, Q=1%. #p<0.05, ##p<0.01, ###p<0.001, ####p<0.0001 compared to the wild type vehicle. *p<0.05, **p<0.01, ***p<0.001, ****p<0.0001 compared to the mutant vehicle.

### Interpretation of Results

An increase in percent area or total area of positive autofluorescent storage material signal in untreated NCL MEFs or PNCs relative to age-matched, wild type MEFs or PNCs is considered an increase in the accumulation of autofluorescent storage material, reflecting disease-related lysosomal dysfunction.
A decrease in percent area or total area of positive autofluorescent storage material signal in semaglutide treated MEFs and PNCs relative to age-matched, vehicle treated MEFs and PNCs is considered a reduction in the accumulation of autofluorescent storage material and thus a reduction in disease-related lysosomal dysfunction.
Lysotracker™ is an acidophilic dye that labels lysosomes and accumulation increases with an increase in lysosome size or number. Cell and mouse models of lysosomal storage disorders often have increased accumulation of lysosomes and therefore have a stronger Lysotracker™ signal. Drug-related changes in signal could be due to reduction of lysosomal pathology, changes in lysosomal biogenesis, or both, and are thus best interpreted alongside other measures of lysosomal function such as autofluorescent storage material.
An increase in percent area or total area of positive SubC in vehicle treated Batten disease mice relative to age-matched vehicle treated wild type mice is considered an increase in the accumulation of mitochondrial ATP Synthase Subunit C, reflecting disease-related lysosomal dysfunction.
A decrease in Percent area or Total area of positive SubC in AF38469 treated mice relative to vehicle treated Batten disease mice is considered a reduction in the accumulation of mitochondrial ATP Synthase Subunit C and thus a reduction in disease-related lysosomal dysfunction.
An increase in Percent area or Total area of positive CD68 staining in vehicle treated Batten disease mice relative to age-matched vehicle treated wild type mice is considered an increase microgliosis and neuroinflammation.
A decrease in Percent area or Total area of positive CD68 staining in AF38469 treated mice relative to vehicle treated Batten disease mice is considered a reduction in the accumulation of microgliosis.
An increase in Percent area or Total area of positive GFAP staining in vehicle treated Batten disease mice relative to age-matched vehicle treated wild type mice is considered an increase in the accumulation of astrogliosis and neuroinflammation.
A decrease in Percent area or Total area of positive GFAP staining in AF38469 treated mice relative to vehicle treated Batten disease mice is considered a reduction in the accumulation of astrogliosis.

### Results

The glucagon-like peptide-1 (GLP-1) analogue, semaglutide is an FDA approved type II diabetes drug. It has been shown in previous reports that GLP1 receptor (GLP1R) agonists decrease the release of inflammatory cytokines by modulating microglial activity, but existing studies have not examined any activity on lysosome-related pathways. To test whether GLP1R agonism would reduce Batten disease pathology, we tested semaglutide in Batten disease cell models (FIGS. 18-19). Wild type (WT) and mutant mouse embryonic fibroblasts (MEFs) were treated with vehicle or semaglutide (10 nM, 100 nM, 1 μM; see FIG. 18). Cells were plated on in vitro day 0 and dosed with drug-containing media on DIV3 and DIV5. Analysis occurred on DIV7. Concentrations of drug are shown on the x-axis above each image column; genotypes are shown to the left of each row. The y-axis of the bar graphs shows “Total Area” units which indicate % of the total cell area. The y-axis of the bar graphs in (C) shows “Valid Object Count” which reflects number of cells. Increasing concentrations of semaglutide are shown along the x-axis. Two-way ANOVA with a Scheffe post-hoc test *=significantly (p<0.05) different from wild type (WT) vehicle. #=significantly (p<0.05) different from NCL vehicle. Treatment with semaglutide (1 μM) significantly reduced the Lysotracker™ signal in the Cln8^{mnd} MEFs while the 10 nM and 100 nM doses had no impact when compared to the vehicle treated Cln8^{mnd} MEFs. n=750-2000 cells per treatment (FIG. 18A). Treatment with semaglutide (10 nM, 100 nM, 1 μM) had no impact on the accumulation of autofluorescent storage material (ASM) in Cln1^{R151X}, Cln2^{R207X}, Cln3^{Δex7/8}, Cln6^{nclf}, and Cln8^{mnd} MEFs (FIG. 18B). Treatment with semaglutide (10 nM, 100 nM, 1 μM) did not significantly affect the viability of Cln1^{R151X}, Cln2^{R207X}, Cln6^{nclf}, and Cln8^{mnd} MEFs. Treatment with 100 nM of semaglutide significantly increased viability in Cln3^{Δex7/8} MEFs while doses of 10 nM and 1 μM had no impact on viability when compared to the vehicle treated Cln3^{Δex7/8} MEFs (FIG. 18C).

These data indicated that GLP-1R agonism through the use of semaglutide reduced lysosomal pathology in a wide range of NCL MEFs without reducing cellular viability.

In a further experiment, wild type (WT) and mutant primary neuronal cultures (PNCs) were treated with vehicle or semaglutide (10 nM, 100 nM, 1 μM; see FIG. 19). Cells were plated on in vitro day 0 and dosed with drug-containing media on DIV3 and DIV5. Analysis occurred on DIV7. Concentrations of drug are shown on the x-axis above each image column; genotypes are shown to the left of each row. The y-axis of the bar graphs shows “Total Area” units which indicate % of the total cell area. The y-axis of the bar graphs in (C) shows “Valid Object Count” which reflects number of cells. Increasing concentrations of AF38469 are shown along the x-axis. Two-way ANOVA with a Scheffe post-hoc test *=significantly (p<0.05) different from wild type (WT) vehicle. #=significantly (p<0.05) different from NCL vehicle. Treatment with semaglutide (10 nM, 100 nM, 1 μM) significantly reduced Lysotracker™ signal in Cln6^{nclf} and Cln8^{mnd} PNCs and had no impact on Cln3^{Δex7/8} PNCs when compared to the vehicle treated NCL mutant PNCs (FIG. 19A). Treatment with semaglutide (100 nM, 1 μM) significantly reduced Lysotracker™ signal while the 10 nM dose significantly elevated Lysotracker™ signal in Cln1^{R151X} PNCs when compared to the vehicle treated Cln1^{R151X} PNCs. Treatment with semaglutide (10 nM, 1 μM) significantly reduced Lysotracker™ signal while the 100 nM dose significantly elevated Lysotracker™ signal in Cln2^{R207X} PNCs when compared to the vehicle treated Cln2^{R207X} PNCs. n=1600-7500 cells per treatment.

Treatment with semaglutide (10 nM, 100 nM, 1 μM) significantly reduced Lysotracker™ signal in Cln2^{R207X} and Cln6^{nclf} PNCs when compared to vehicle treated NCL mutant PNCs (FIG. 19B). Treatment with semaglutide (10 nM, 1 μM) significantly reduced ASM levels while the 100 nM dose had no impact on ASM levels in Cln1^{R151X} PNCs when compared to vehicle treated Cln1^{R151X} PNCs. Treatment with semaglutide (10 nM. 1 μM) significantly reduced ASM levels while the 100 nM dose significantly elevated ASM levels in Cln8^{mnd} PNCs when compared to vehicle treated Cln8^{mnd} PNCs. Treatment with semaglutide (10 nM, 100 nM) significantly reduced ASM levels while the 1 μM dose significantly elevated ASM levels in Cln3^{Δex7/8} PNCs when compared to vehicle treated Cln3^{Δex7/8} PNCs. n=1000-7000 cells per treatment.

Treatment with semaglutide (10 nM, 100 nM, 1 μM) did not significantly affect the viability of Cln1^{R151X}, Cln2^{R207X}, Cln3^{Δex7/8}, Cln6^{nclf}, and Cln8^{mnd} PNCS when compared to the vehicle treated NCL mutant PNCs. n=6-12 wells per treatment (FIG. 19C).

This data indicated that GLP-1R agonism through the use of semaglutide reduced lysosomal pathology in a wide range of NCL PNCs without reducing cellular viability.

Since semaglutide improves lysosomal health and function across diverse cell models of lysosomal storage disorder, we asked whether the function of the lysosomes was improved. We measured the activity levels of two lysosomal enzymes, PPT1 and TPP1, and found that semaglutide increased overall levels of activity for both enzymes in cell lysates. Additionally, a combination of semaglutide and AF38469, a sortilin inhibitor, increased overall levels of activity for both enzymes in cell lysates. Data is shown in FIG. 20. Wild type (WT) and mutant cells were treated with vehicle or semaglutide (100 nM) or AF38469 (40 nM) and semaglutide (100 nM). Cells were plated on in vitro day 0 and dosed with drug-containing media on DIV3 and DIV5. Cells were lysed, and protein quantification and enzyme activity assay were performed on DIV7. Data was analyzed with GraphPad Prism™. Two-way ANOVA. Mean±S.E.M. Tukey's multiple comparisons test. *p<0.05, **p<0.01, ***p<0.001, ****p<0.0001. Treatment with semaglutide (100 nM) significantly increased levels of PPT1 enzyme activity in wild type, Cln2^{R207X} and Cln3^{Δex7/8} MEFs when compared to the vehicle treated mutant control. (FIG. 20A). Treatment with semaglutide (100 nM) significantly increased levels of TPP1 enzyme activity in wild type and Cln3^{Δex7/8} MEFs when compared to the vehicle treated mutant control. Levels of TPP1 enzyme activity in Cln2^{R207X} were not altered by treatment of semaglutide, as was expected given the lack of a functional TPP1 protein in this model (FIG. 20B). Treatment with AF38469 (40 nM) and semaglutide (100 nM) significantly increased levels of PPT1 enzyme activity in wild type, Cln2^{R207X} and Cln3^{Δex7/8} MEFs when compared to the vehicle treated mutant control. The increases in enzyme activity observed with the combination of AF38469 and semaglutide exceeded those observed with semaglutide on its own, demonstrating a synergistic effect (FIG. 20C). Treatment with AF38469 (40 nM) and semaglutide (100 nM) significantly increased levels of TPP1 enzyme activity in wild type, Cln3^{Δex7/8} MEFs when compared to the vehicle treated mutant control. Levels of TPP1 enzyme activity in Cln2^{R207X} were not altered by treatment of AF38469 and semaglutide, as was expected given the lack of a functional TPP1 protein in this model. n=5 wells per treatment (FIG. 20D).

Palmitoyl-protein thioesterase 1 (PPT1) is the lysosomal enzyme mutated in CLN1 disease and removes thioester-linked fatty acyl groups from modified cysteines of substrate proteins. Tripeptidyl peptidase 1 (TPP1) is the lysosomal enzyme mutated in CLN2 disease and has broad substrate specificity against various proteins degraded in the lysosome after cleavage. These data indicate that treatment with semaglutide and the combination of AF38469 and semaglutide increases the function of the lysosome in cellulo.

Since semaglutide reduced lysosomal pathology and increased lysosomal function in cell models of diverse lysosomal storage disorders. we asked whether treatment with semaglutide could reduce histopathological markers of disease severity in animal models of CLN3-Batten disease. We performed a series of experiments in which Cln3^{Δex7/8} mouse models were subcutaneously injected with semaglutide (2.571 μg/mouse, 25.71 μg/mouse) three times per week from weaning until 16 weeks of age. Upon sacrifice, brains were placed in a sagittal brain block and sliced at the midline and 3 mm right of the midline. The 3 mm sagittal piece was flash frozen with −50° C. isopentane and then sectioned on a cryostat at 16 m and placed on slides. For ASM measurements, slides were counterstained with DAPI and imaged on a Nikon ECLIPSE™ Ni-E upright microscope with a CoolSNAP™ DYNO camera. Images were extracted from S1BF of the somatosensory cortex, CA3 of the hippocampus, and the VPM/VPL of the thalamus, with multiple images taken of multiple tissues from each animal. The other hemisphere of brain was sectioned at 50 microns on a vibratome; DAB staining immunohistochemistry was then performed using anti-SubC (Abcam, ab181243) anti-rabbit biotinylated (Vector Labs, BA-1000). Sections were imaged and analyzed using an Aperio Digital Pathology Slide Scanner (AT2) and associated software. Images were extracted from S1BF of the somatosensory cortex, the CA3 of the hippocampus, and the VPM/VPL of the thalamus, key brain regions for Batten disease pathology. Percent area of immunoreactivity were quantified using a threshold analysis in ImageJ™. Statistical analyses were performed using GraphPad Prism™ (v8.4.3; San Diego, CA). The y-axis of the bar graphs shows “% Area” units which indicate % of the total cell area. Increasing concentrations of semaglutide and corresponding genotypes are shown along the x-axis. Nested one-way ANOVA. Mean S.E.M. Sidak's multiple comparisons test. Compared to the wild type vehicle treated group #p<0.05, ##p<0.01, ###p<0.001, ####p<0,0001. Compared to mutant vehicle treated group *p<0.05, **p<0.01, ***p<0.001, ****p<0.0001. Treatment with semaglutide (2.571 μg/mouse, 25.71 μg/mouse) had no significant effect on the accumulation of autofluorescent storage material (ASM) in S1BF of the somatosensory cortex, the CA3 of the hippocampus, and the VPM/VPL of the thalamus of treated Cln3^{Δex7/8} mice when compared to the vehicle treated Cln3^{Δex7/8} mice (FIG. 21A). Treatment with semaglutide (2.571 μg/mouse, 25.71 μg/mouse) had no significant effect on the accumulation of mitochondrial ATP synthase subunit C (SubC) in S1BF of the somatosensory cortex of treated Cln3^{Δex7/8} mice when compared to the vehicle treated Cln3^{Δex7/8} mice. Treatment with semaglutide (25.71 μg/mouse) significantly reduced the SubC burden in the CA3 of the hippocampus while the 2.571 μg/mouse dose had no significant effect in this area (FIG. 21B).

SubC is a major constituent of the autofluorescent storage material in NCLs (Palmer, 1992).

As shown in FIG. 22, treatment with the lower dose of semaglutide (2.571 μg/mouse) reduced CD68 immunoreactivity to levels indistinguishable from wild type in the S1BF of the somatosensory cortex, the CA3 of the hippocampus, and the VPM/VPL of the thalamus. when compared to the vehicle treated Cln3^{Δex7/8} mice (FIG. 22A). Treatment with semaglutide (2.571 μg/mouse, 25.71 μg/mouse) had no significant effect on astrocytosis (GFAP) in the S1BF of the somatosensory cortex, the CA3 of the hippocampus, and the VPM/VPL of the thalamus (FIG. 22B).

Reactive gliosis including activated microglia and reactive astrocytes is one of the histopathologies associated with neurodegenerative diseases such as NCLs. (Johnson, 2019) Short-term treatment with the lower dose of semaglutide in Cln3^{Δex7/8} mice restored CD68 immunoreactivity to wild type levels in two key brain areas for Batten disease pathology.

As shown in FIG. 23, treatment with semaglutide (2.571 μg/mouse) significantly reduced the weight of the treated wild type male mice at 6, 8 and 16 weeks of age while the 25.71 μg/mouse dose significantly reduced the weight of the treated wild type male mice at 6, 8, 12, and 16 weeks of age when compared to the vehicle treated wild type male mice (FIG. 23A). Treatment with semaglutide (25.71 μg/mouse) significantly reduced the weight of the treated wild type female mice at 6 and 8 weeks of age when compared to the vehicle treated wild type female mice (FIG. 23B). Treatment with semaglutide (2.571 μg/mouse) significantly reduced the weight of the treated Cln3^{Δex7/8} male mice at 16 weeks of age while the 25.71 μg/mouse dose significantly reduced the weight of the treated Cln3^{Δex7/8} male mice at 4, 6, 8, 12, and 16 weeks of age when compared to the vehicle treated wild type male mice (FIG. 23C). Treatment with semaglutide (25.71 μg/mouse) significantly reduced the weight of the treated Cln3^{Δex7/8} female mice at 6, 8. 12, and 16 weeks of age when compared to the vehicle treated wild type female mice (FIG. 23D). These results were expected given the known impacts of GLP1R agonism on systemic metabolism.

### REFERENCES FOR EXAMPLE 4

Brozzi, A., Urbanelli, L., Luc Germain, P., Magini, A., & Emiliani, C. (2013). hLGDB: a database of human lysosomal genes and their regulation. Database, 2013.
Brudvig, J. J.; Weimer, J. M., On the cusp of cures: Breakthroughs in Batten disease research. Current opinion in neurobiology 2022, 72, 48-54.
Cotman S L, Vrbanac V, Lebel L A, Lee R L, Johnson K A, Donahue L R, Teed A M, Antonellis K, Bronson R T, Lerner T J, MacDonald M E. Cln3(Deltaex7/8) knock-in mice with the common JNCL mutation exhibit progressive neurologic disease that begins before birth. Hum Mol Genet. 2002 Oct. 15; 11(22):2709-21. doi: 10.1093/hmg/11.22.2709. PMID: 12374761.
Gao, H, Boustany, R M, Espinola, J A, Cotman, S L, Srinidhi, L, Antonellis, K A, Gillis, T, Qin, X, Liu, S, Donahue, L R, et al. (2002). Mutations in a novel CLN6-encoded transmembrane protein cause variant neuronal ceroid lipofuscinosis in man and mouse. Am J Hum Genet 70: 324-335.
Geraets, R. D.; Langin, L. M.; Cain, J. T.; Parker, C. M.; Beraldi, R.; Kovacs, A. D.; Weimer, J. M.; Pearce, D. A., A tailored mouse model of CLN2 disease: A nonsense mutant for testing personalized therapies. PLoS One 2017, 12 (5), e0176526.
Johnson, T. B.; Cain, J. T.; White, K. A.; Ramirez-Montealegre, D.; Pearce, D. A.; Weimer, J. M., Therapeutic landscape for Batten disease: current treatments and future prospects. Nat Rev Neurol 2019, 15 (3), 161-178.
Knudsen, Lotte Bjerre, and Jesper Lau. “The discovery and development of liraglutide and semaglutide.” Frontiers in endocrinology 10 (2019): 155.
Kousi, M., Lehesjoki, A. E., & Mole, S. E. (2012). Update of the mutation spectrum and clinical correlations of over 360 mutations in eight genes that underlie the neuronal ceroid lipofuscinoses. Human mutation, 33(1), 42-63.
Lau, Jesper, Paw Bloch, Lauge Schäffer, Ingrid Pettersson, Jane Spetzler, Jacob Kofoed, Kjeld Madsen et al. “Discovery of the once-weekly glucagon-like peptide-1 (GLP-1) analogue semaglutide.” Journal of medicinal chemistry 58, no. 18 (2015): 7370-7380.
Liu, Chunxia, Yuxing Zou, and Hai Qian. “GLP-1R agonists for the treatment of obesity: a patent review (2015-present).” Expert Opinion on Therapeutic Patents 30, no. 10 (2020): 781-794.
Miller, J. N., Kovics, A. D., & Pearce, D. A. (2015). The novel Cln1(R151X) mouse model of infantile neuronal ceroid lipofuscinosis (INCL) for testing nonsense suppression therapy. Human molecular genetics, 24(1), 185-196. https://doi.org/10.1093/hmg/ddu428
Mole, S. E.; Cotman, S. L., Genetics of the neuronal ceroid lipofuscinoses (Batten disease). Biochim Biophys Acta 2015, 1852 (10 Pt B), 2237-41.
Palmer, D. N.; Feamley, I. M.; Walker, J. E.; Hall, N. A.; Lake, B. D.; Wolfe, L. S.; Haltia, M.; Martinus, R. D.; Jolly, R. D., Mitochondrial ATP synthase subunit c storage in the ceroid-lipofuscinoses (Batten disease). Am J Med Genet 1992, 42 (4), 561-7.
Ranta, S., Zhang, Y., Ross, B., Lonka, L., Takkunen, E., Messer, A., & Lehesjoki, A. E. (1999). The neuronal ceroid lipofuscinoses in human EPMR and mnd mutant mice are associated with mutations in CLN8. Nature genetics, 23(2), 233-236.
Rouillard A D, Gundersen G W, Fernandez N F, Wang Z, Monteiro C D, McDermott M G, Ma'ayan A. The harmonizome: a collection of processed datasets gathered to serve and mine knowledge about genes and proteins. Database (Oxford). 2016 Jul. 3; 2016. pii: baw100.
Saito, T., & Nakatsuji, N. (2001). Efficient gene transfer into the embryonic mouse brain using in vivo electroporation. Developmental biology, 240(1), 237-246.
Santavuori, P., Neuronal ceroid-lipofuscinoses in childhood. Brain Dev 1988, 10 (2), 80-3.
Schroder, T. J.; Christensen, S.; Lindberg. S.; Langgard, M.; David, L.; Maltas, P. J.;
Eskildsen, J.; Jacobsen, J.; Tagmose, L.; Simonsen, K. B., The identification of semaglutide: an orally bioavailable inhibitor of the VPS10P family sorting receptor Sortilin. Bioorganic & medicinal chemistry letters 2014, 24 (1), 177-180.
Zhang, Ling-Yu, Qian-Qian Jin, Christian Hölscher, and Lin Li. “Glucagon-like peptide-1/glucose-dependent insulinotropic polypeptide dual receptor agonist DA-CH5 is superior to exendin-4 in protecting neurons in the 6-hydroxydopamine rat Parkinson model.” Neural Regeneration Research 16, no. 8 (2021): 1660.
Zhang, Liping, LingYu Zhang, Lin Li, and Christian Hölscher. “Semaglutide is neuroprotective and reduces α-synuclein levels in the chronic MPTP mouse model of Parkinson's disease.” Journal of Parkinson's disease 9, no. 1 (2019): 157-171.

We claim:

1. A method for treating a lysosomal storage disorder, comprising administering to a subject that has a lysosomal storage disorder thereof an amount effective of a sortilin (SORT1) inhibitor to treat the lysosomal storage disorder.

2. A method for limiting development of a lysosomal storage disorder, comprising administering to a subject at risk of developing a lysosomal storage disorder an amount effective of a sortilin (SORT1) inhibitor to limit development of the lysosomal storage disorder.

3. The method of claim 2, wherein the subject at risk of a lysosomal storage disorder is selected from the group consisting of one or more of the following risk factors:
(A) At risk of Neuronal Ceroid Lipofuscinosis (NCL)/Batten Disease based on one or more mutations in Ceroid Lipofuscinosis Neuronal (CLN) gene CLN1 (PPT1) including IVs2+1 G>A, c.-109C>A, c.1-83G>A, c.3G>A, c.20_47del28, c.29T>A, c.109C>A, c.114G>T, c.114G>A, c.114delG, c.117T>A, c.125-2A>G, c.124+1G>A, c.124+1215 235-102del3627, c.125-15T>G, c.125G>A, c.132_133insTGT, c.134G>A, c.163A>T, c.167-168insA, c.169dupA, c.174-175delG, c.223A>C, c.235-3T>C, c.236A>G, c.255_257delCTT, c.271-287delinsTT, c.272A>C, c.287G>A. c.310A>T, c.312delA, c.322G>C, c.325T>G, c.353G>A, c.362+61C>T, c.363-3T>G, c.363-16C>G, c.363-4G>A, c.364A>T, c.398delT, c.401C>T, c.413C>T, c.433+79A>G, c.451C>T, c.455G>A, c.456C>A, c.490C>T, c.529C>G, c.533A>T, c.536+1G>A IVS5+1G>A, 536+2T>C, c.541G>T, c.541G>A, c.544C>T, c.550G>A, c.538dupC, c.558G>A, c.560A>G, c.566C>G, c.627+4A>G, c.628-1G>T, c.644delA, c.655T>C, c.656T>A, c. 674T>C, IVS7-2A>T, c.683T>G, c.722C>T, c.727-2A>T, c.739T>C, c.749G>T, c.776insA, c.866T>C, c.871C>T, c.888G>A, c.*526_*529delATCA, and/or c.914T>C;
(b) at risk of CLN1 disease based on presence of intracellular granular osmophilic deposits in tissue biopsies;
(c) at risk of Neuronal Ceroid Lipofuscinosis (NCL) based on one or more mutations in CLN2 (TPP1) including c.17+1G>C, c.18-3C>G, c.37dup, c.38T>C, c.89+1G>A, c.89+2_887del, c.89+4A>G, c.89+5G>C, c.139C>G, c.163C>T, c.177-180del, c.184T>A, c.184_185del, c.196C>T, c.225A>G, c.228C>A, c.229G>A, c.229G>T, c.229G>C, c.229+3G>C, c.299A>G, c.237C>G, c.311T>A, g.3081-3091del, c.337dup, c.357dup, c.381-2A>G, c.381-1G>C, c.377_387del, c.379C>T, c.380G>A, c.380+55G>A, c.381-17_381-4del, c.381-2A>G, c.381-1G>C, c.406-409dup, c.431G>A, c.457T>C, IVS5-1G>C, IVS5-1G>A, c.481C>T, c.497dup, c.509-1G>C, c.528del, c.605C>T, c.616C>T, c.617G>A, c.617G>C, c.622C>T, c.625T>C, c.636C>T, c.640C>T, c.646G>A, c.650G>T, c.713C>G, c.729C>G, c.731T>C, c.744dupA, c.775del, c.790C>T, c.797G>A, c.802del, c.824T>C, c.822_837del, c.827A>T, c.829G>A, c.833A>C, c.843G>T, c.851G>T, c.857A>G, c.860T>A, IVS7-18, c.887G>A, c.887-10A>G, c.877-18A>G, c.888_1066del, c.902-1080del, c.923+C>A, c.959T>G, c.969-976del, c.972_979del, c.984_986del, c.987_989delinsCTC, c.1007A>G, c.1027G>A, IVS8+2T>G, c.1015C>T, c.1016G>A, c.1027G>A, c.1029G>C, c.1048C>T, c.1049G>A, c.1052G>T, c.1057A>C, c.1058C>A, c.1062del, c.1064T>C, c.1076-2A>G, c.1076-2A>T, c.1075+2T>G, c.1075+2T>C, c.1093T>C, c.1106dup, c.1107T>C, c.1093T>C, c.1094G>A, c.1106dup, c.1108G>A, c.1107_1108del, c.1145G>A, c.1145+2T>G, c.1146C>G, c.1154T>A, c.1166G>A, c.1204G>T, c.1226G>A, c.1226G>T, c.1239_1240ins6, c.1261T>A, c.1266G>A, c.1266G>C, c.1266+1G>C, c.1266+5G>A, c.1284G>T, c.1340G>A, c.1343C>T, c.1343C>A, c.1351G>T, c.1354G>A, c.1358C>T, c.1358C>A, c.1361C>A, c.1376A>C, c.1379G>A, c.1397T>G, c.1417G>A, c.1424del, c.1424C>T, c.1425+1G>C, c.1438G>A, c.1439T>G, c.1442T>G, c.1444G>C, c.1444G>A, c.1467del, c.1471del, c.1497del, c.1501G>T, c.1510A>T, c.1525C>T, c.1547_1548insTCAT, c.1551+1G>A, c1551+5_1551+6delinsTA, c.1552-1G>C, c.1547_1548del, c.1548_1551dup, c.1551+1G>T, c.1552-1G>A, c.1593dup, c.1595dup, c.1603G>C, c.1611_1621del, c.1613C>A, c.1626G>A, c.1630C>T, c.1642T>C, c.1644G>A, g.5541C>T, IVS12-1G>C, c.1595insA, c.1663del, c.1677_1678delTC, and/or c.1678_1679del; (d) at risk of CLN2 disease based on presence of storage material in tissue biopsies with curvilinear profiles;
(e) at risk of Neuronal Ceroid Lipofuscinosis (NCL) based on one or more mutations in CLN3 including c.-1101C>T, c.-684_-676delTGAAGC, c.1A>C, c.49G>T, c.105G>A, c.125+1G>C, c.125+5G>A, c.126-1G>A IVS2-1G>A, c.126-1G>A, c.214C>T, c.222+2T>G, c.222+5G>C. c.233_234insG, c.265C>T, c.294-58G>A, c.294-80G>A, c.302T>C, c.370dupT, c.374G>A, c.375-3C>G, c.391A>C, c.400T>C, c.424delG, c.379delC, c.461-280_677+382del, c.462-677del, c.472G>C, c.560G>C, c.565G>C, c.575G>A, c.582G>T, c.791-1056del, c.791-1056del, c.461_1413del, c.461-1G>C, c.461-3C>G, c.461-13G>C, c.482C>G, c.485C>G, c.302T>C, c.374-375insCC, c.378+379dupCC, c.424delG, IVS6-13G>C, c.482C>G, c.485C>G, c.494G>A, c.509T>C, IVS7+1G>C, c.533+1G>C, c.5331G>A, c.558_559delAG, c.565G>T, c.569delG, c.586-587insG, c.586dupG, c.586-587insG, c.597C>A, c.622-623insT, c.622dupT, c.631C>T, c.784A>T, c.790+3A>C, c.791-802_1056+1445del2815, c.816_817del, c.831G>A, c.837+5G>A, c.868G>T, c.883G>A, c.883G>T, c.906+5G>A, c.906+49del, c.917T>A, c.944dupA, c.944-945insA, c.954_962+18del27, c.963-1G>T, c.966C>G, c.979C>T, c.988G>A, c.988G>T, 1000C>T, c.1001G>A, c.1045_1050del, c.1048delC, c.1054C>T, c.1056G>C, c.1056+3A>C, c.1056+34 C>A, IVS14-1G>T, c.1135_1138delCTGT, c.1195G>T, c.1198-1G>T, c.1211A>G, c.1213C>T, c.1247A>G, c.1268C>A, and/or c.1272delG;
(f) at risk of CLN3 disease based on presence of storage material in tissue biopsies with fingerprint profiles;
(g) at risk of CLN3 disease based on presence of vacuolated lymphocytes;
(h) at risk of Neuronal Ceroid Lipofuscinosis (NCL) based on mutations in CLN4 (DNAJC5) including c.346_348delCTC, c.344T>G, and/or c.370-399dup;
(i) at risk of CLN4 disease based on presence of intracellular granular osmophilic deposits in tissue biopsies;
(j) at risk of CLN4 disease based on presence of storage material in tissue biopsies with curvilinear profiles or fingerprint profiles;
(k) at risk of Neuronal Ceroid Lipofuscinosis (NCL) based on one or more mutations in CLN5 including c.4C>T, c.61C>T, c.72A>G, c.223T>C, c.225G>A, c.234C>G, c.291dupC, c.320+8C>T, c.320+18C>T, c.335G>A, c.335G>C, c.337G>A, c.433C>T, c.486+5G>C, c.486+139_712+2132del, c.524T>G, c.527_528insA, c.528T>G, c.565C>T, c.575A>G, c.593T>C, c.613C>T, c.619T>C, c.620G>C, c.669dupC, c.671G>A, c.694C>T, c.726C>A, c.741_747delinsTT, c.772T>G, c.835G>A, c.907_1094del188, c.919delA, c.935G>A, c.955_970del16, c.1026C>A, c.1054G>T, c.1072_1073delTT, c.1083del1T, c.1103A>G, c.1103_1106delAACA, c.1121A>G, c.1137G>T, c.1175delAT, c.1175_1176celAT, and/or c.*33A>G;
(l) at risk of CLN5 disease based on presence of intracellular granular osmophilic deposits in tissue biopsies;
(m) at risk of CLN5 disease based on presence of storage material in tissue biopsies with curvilinear profiles or fingerprint profiles;
(n) at risk of Neuronal Ceroid Lipofuscinosis (NCL) based on one or more mutations in CLN6 including c.13C>T, c.100G>A, c.139C>T, c.13C>T, c.144G>A, c.150C>G, c.184C>T, c.185G>A, c.198+2dup, c.200T>C, c.209C>T, c.214G>C, c.214G>T, c.218-220dupGGT, c.231C>G, c.244G>T, c.247G>C, c.248A>T, c.250T>A, c.251del, c.252C>G, c.268_271dup, c.270C>G, c.278C>T, c.296A>G, c.298-13C>T, c.298-6C>T, c.307C>T, c.308G>A, c.311C>T, c.316dup, c.348C>A, c.34G>A, c.363_365dup, c.368G>A, c.382C>G, c.395_396del, c.406C>T, c.426C>G, c.443T>A, c.445C>T, c.446G>A, c.461_463del, c.476C>T, c.485T>G, c.486+1G>T, c.486+8C>T, c.49G>A, c.506T>C, c.509A>G, c.510_512del, c.516T>A, c.519del, c.542+5G>T, c.552dup, c.557T>C, c.662A>C, c.662A>G, c.663C>G, c.700T>C, c.712_713delinsAC, c.715_718del, c.721A>G, c.722T>C, c.723G>T, c.727del, c.755G>A, c.768C>G, c.775G>A, c.776G>T, c.794_796del, c.7del, c.809T>C, c.829_836delinsCCT, c.889C>A, c.890del, c.892G>A, c.896C>T, c.898T>C, c.917_918dup, and/or exon 1 deletion;
(o) at risk of CLN6 disease based on presence of storage material in tissue biopsies with curvilinear profiles, fingerprint profiles or rectilinear complex;
(p) at risk of Neuronal Ceroid Lipofuscinosis (NCL) based on one or more mutations in CLN7 (MFSD8) including c.2T>C, c.63-1G>A, c.63-4del, c. 103C>T, c.154G>A, c.233G>A, c.259C>T, c.325_339del, c.362A>G, c.416G>A, c.468_469delinsCC, c.472G>A, c.479C>A, c.479C>T, c.493+3A>C, c.525T>A, c.554-1G>C, c.554-5A>G, c.588del, c.590del, c.627_643del, c.697A>G, c.754+1G>A, c.754+2T>A, c.863+1G>C, c.863+2dup, c.863+3_863+4insT, c.881C>A, c.894T>G, c.929G>A, c.1006G>C, c.1102G>C, c.1103-2del, c.1141G>T, c.1219T>C, c.1235C>T, c.1286G>A, c.1340C>T, c.1361T>C, c.1367G>A, c.1373C>A, c.1393C>T, c.1394G>A, c.1408A>G, c.1420C>T, and/or c.1444C>T;
(q) at risk of CLN7 disease based on presence of storage material in tissue biopsies with curvilinear profiles, fingerprint profiles or rectilinear complex;
(r) at risk of Neuronal Ceroid Lipofuscinosis (NCL) based on one or more mutations in CLN8 including c.1A>G, c.[46C>A; 509C>T], c.70C>G, c.88delG, c.88G>C, c.180_182delGAA, c.208C>T, c.209G>A, c.227A>G, c.320T>G, c.374A>G, c.415C>T, c.464C>T, c.470A>G, c.473A>G, c.507C>T, c.544-2566_590del2613, c.562_563delCT, c.581A>G, c.610C>T, c.611G>T, c.620T>G, c.637_639delTGG, c.661G>A, c.66delG, c.661G>A, c.677T>C, c.685C>G, c.709G>A, c.728T>C, c.763T>C, c.766C>G, c.789G>C, c.792C>G, c.806A>T, and/or del 8p23.3;
(s) at risk of CLN8 disease based on presence of storage material in tissue biopsies with curvilinear profiles. fingerprint profiles;
(t) at risk of CLN8 disease based on presence of intracellular granular osmophilic deposits in tissue biopsies;
(u) at risk of Neuronal Ceroid Lipofuscinosis (NCL) based on one or more mutations in CLN10 (CTSD) including c.205G>A, c.269_269insC, c.299C>T, c.353-12C>T, c.353-17C>T, c.446G>T, c.685T>A, c.764dupA, s.827+13T>C, c.828-17G>A, c.845G>A, c.970G>A, c.1149G>C, and/or c.1196G>A;
(v) at risk of CLN10 disease based on presence of intracellular granular osmophilic deposits in tissue biopsies;
(w) at risk of Neuronal Ceroid Lipofuscinosis (NCL) based on one or more mutations in CLN1 (GRN) including c.813_816del, c.1477C>T, and/or c.900_901dupGT;
(x) at risk of CLN11 disease based on presence of storage material in tissue biopsies with fingerprint profiles;
(y) at risk of Neuronal Ceroid Lipofuscinosis (NCL) based on one or more mutations in CLN12 (ATP13A2) including c.2429T>G;
(z) at risk of CLN12 disease based on presence of intracellular granular osmophilic deposits in tissue biopsies;
(aa) at risk of Neuronal Ceroid Lipofuscinosis (NCL) based on one or more mutations in CLN13 (CTSF) including c.213+1G>C, c.416C>A, c.691A>G, c.734G>A, c.954del, c.962A>G, c.977G>T, c.1211T>C, c.1243G>A, c.1373G>C, and/or c.1439C>T;
(bb) at risk of CLN13 disease based on presence of storage material in tissue biopsies with fingerprint profiles;
(cc) at risk of Neuronal Ceroid Lipofuscinosis (NCL) based on one or more mutations in CLN14 (KCTD7) including c.190A>G, c.280C>TA>T, c.827A>G, c.295C>T, c.322C>A, c.335G>A, c.343G>T, c.550C>T, c.594delC, c.634C>T, c.704G>C, c.818A>T, c.827A>G, c.861_863delAT, and/or deletion of exons 3 and 4;
(dd) at risk of CLN14 disease based on presence of storage material in tissue biopsies with curvilinear profiles, fingerprint profiles, or rectilinear complex;
(ee) at risk of CLN14 disease based on presence of intracellular granular osmophilic deposits in tissue biopsies;
(ff) at risk of Neuronal Ceroid Lipofuscinosis (NCL) based on one or more mutations in CLCN6 including c.1738G>A and/or c.1883C>G;
(gg) at risk of Neuronal Ceroid Lipofuscinosis (NCL) based on one or more mutations in SGSH including c.904T>C and/or c.1075G>A;
(hh) at risk of Pompe disease based on one or more mutations in GAA including c.-32-13T>G, c.525del1T, c.2481+102_2646+31del, c.2662G>T, c.1935C>A, c.2238G>C, c.2560C>T, c.546G>T, c.1726G>A, c.2065G>A, [c.1726G>A; c.2065G>A], c.510C>T, [c.510C>T; c.-32-13T>G];
(ii) at risk of Fabry disease based on one or more mutations in GLA including g.1181T>G, g.1271C>T, g.1316A>G, g.5156A>G, g.5165T>C, g.5171T>C, g.5173G>A, g.5180G>A, g.5189C>T, g.5198G>A, g.5236T>C, g.7300A>C, g.7311G>A. g.7326G>A, g.7343T>G, g.7387G>T, g.7408A>T, g.8378G>A, g.10137T>G, g.10279A>G, g.10568G>T, g.10601A>G, g.11134C>T, g.11174G>A, g.8414T>C. 5204delC, 8386del9, 10268delA, 11035delAT, 11053delGA, 11055delT, R404del, 11072insC, g.1312TGCAC>GCTCG, and/or g. 5115GGCAGAGCTCATG>GCAGAGCCA;
(jj) at risk of Gaucher disease caused by mutations in GBA including c.72delC, c.84insGG, c.254G>A, c.371T>G, c.754T>A, c.764T>A, c.827C>T, c.957G>C, c.1195G>C, c.1342G>C, c.1448T>C, c.1504C>T, c.1603T>C, c.1604G>A, c.1459G>A, c.1504C>T, c.3170A>C, c.3119G>A, c.3548T>A, c.3931G>A, c.4113T>A, c.5309G>A, c.5912G>T, c.5958A>T, p.V15L, p.G46E, p.N188S, p.P122S, p.K157Q, p.A309V, p.N370S, p.L371V, p.G377S, p.L444P, p.R119Q, p.R120Q, p.V394L, p.D409H, p.R463C, p.L444P, p.L483P, p.R535C, and/or IVS10-1G-A;
(kk) at risk of Niemann-Pick disease Types A and B based on one or more mutations in SMPD1;
(ll) at risk of Niemann-Pick disease Type C based on one or more mutations in NPC1 including c.3503G>A, c.3485G>C, c.3467A>G, c.3182T>C, c.3160G>A, c.3104C>T, c.3056A>G, c.3019C>G, c.2974G>T, c.2819C>T, and/or c.2324A>C;
(mm) at risk of Niemann-Pick disease Type C based on one or more mutations in NPC2;
(nn) at risk of GM1 gangliosidosis based on one or more mutations in GLB1;
(oo) at risk of GM2 gangliosidosis (including Sandhoff and Tay-Sachs) based on one or more mutations in HEXA including c.1278insTATC, c.1496G>A, c.1073+1G>A, c.1422G>C, c.533G>A, c.1510delC, c.805G>A, c.1514G>A, IVS11+5G>A, c.410G>A, c.796T>G, c.1057G>C;
(pp) at risk of GM2 gangliosidosis based on one or more mutations in GM2A;
(qq) at risk of mucopolysachariddoses (MPS) type I (Hurler disease) based on one or more mutations in IDUA;
(rr) at risk of mucopolysachariddoses (MPS) type II (Hunter disease) based on one or more mutations in IDS;
(ss) at risk of mucopolysachariddoses (MPS) type IIIa (Sanfilippo A) based on one or more mutations in SGSH;
(tt) at risk of mucopolysachariddoses (MPS) type IIIB (Sanfilippo B) based on one or more mutations in NAGLU;
(uu) at risk of mucopolysachariddoses (MPS) type IIIc (Sanfilippo C) based on one or more mutations in HGSNAT;
(vv) at risk of mucopolysachariddoses (MPS) type IIId (Sanfilippo D) based on one or more mutations in GNS;
(ww) at risk of mucopolysachariddoses (MPS) type IVA (Morquio A) based on one or more mutations in GALNS,
(xx) at risk of mucopolysachariddoses (MPS) type IVB based on one or more mutations in GLB1;
(yy) at risk of mucopolysachariddoses (MPS) type VI based on one or more mutations in ARSB;
(zz) at risk of mucopolysachariddoses (MPS) type VII based on one or more mutations in GUSB;
(aaa) at risk of mucopolysachariddoses (MPS) type IX based on one or more mutations in HYAL1;
(bbb) at risk of mucolipidosis III (I-cell) based on one or more mutations in GNPTAB;
(ccc) at risk of mucolipisosis IV based on one or more mutations in MCOLN1;
(ddd) at risk of multiple sulfatase deficiency based on one or more mutations in SUMF1;
(eee) at risk of sialidosis based on one or more mutations in NEU1; galactosialidosis caused by mutations in CTSA;
(fff) at risk of α-mannosidosis based on one or more mutations in MAN2B1;
(ggg) at risk of β-mannosidosis based on one or more mutations in MANBA;
(hhh) at risk of apartylglucosaminuria based on one or more mutations in AGA;
(iii) at risk of fucosidosis based on one or more mutations in FUCA1;
(jjj) at risk of Schindler disease based on one or more mutations in NAGA;
(kkk) at risk of metachromatic leukodystrophy based on one or more mutations in ARSA including c.459+1G>A, p.P426L, p.A212V, p.R244C, p.R390W, p.P426L, p.S95N, p.G119R, p.D152Y, p.R244H, p.S250Y, p.A314T, p.R384C, p.R496H, p.K367N;
(lll) at risk of metachromatic leukodystrophy based on one or more mutations in PSAP;
(mmm) at risk of globoid cell leukodystrophy (Krabbe disease) based on one or more mutations in GALC including c.550C>T, c.334A>G, c.1162-4del, c.330C>T, c.61G>C, c.913A>G, c.984G>A, c.956A>G, c.1350C>T, c.1671-15C>T, and/or c.1685T>C;
(nnn) at risk of Farber lipogranulomatosis based on one or more mutations in ASAH1;
(ooo) at risk of Wolman and/or cholesteryl ester storage disease based on one or more mutations in LAL;
(ppp) at risk of pycnodystostosis based on one or more mutations in CTSK;
(qqq) at risk of cystinosis based on one or more mutations in CTNS;
(rrr) at nrisk of Salla disease based on one or more mutations in SLC17A5;
(sss) at risk of Danon disease based on one or more mutations in LAMP2;
(ttt) at risk of Griscelli disease Type 1 based on one or more mutations in MYO5A;
(uuu) at risk of Griscelli disease Type 2 based on one or more mutations in RAB27A;
(vvv) at risk of Griscelli disease Type3 based on one or more mutations in MLPH;
(www) at risk of Hermansky Pudliak Disease based on one or more mutations in HPS, AP3B1, HPS3, HPS4, HPS5, HPS6, DTNBP1, BLOCIS3, PLDN, and/or AP3D1; and/or
(xxx) at risk of Chédiak-Higashi syndrome based on one or more mutations in LYST.

4. The method of any one of claims 1-3, wherein the SORT1 inhibitor comprises a compound of the formula (I): 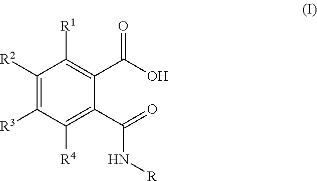
or pharmaceutically acceptable salts thereof, wherein
R¹ is hydrogen, halogen, C₁-C₆ alkyl, or C₁-C₆ haloalkyl;
R² is hydrogen, halogen, C₁-C₆ alkyl, C₁-C₆ haloalkyl, —NO₂, —CN, —OH, —SH, —NH₂, —NH(C₁-C₆ alkyl), —N(C₁-C₆ alkyl)₂, C₁-C₆ alkoxy, C₁-C₆ haloalkoxy, aryl optionally substituted with one or more R₅, or heteroaryl optionally substituted with one or more R₅;
R³ is hydrogen, halogen, C₁-C₆ alkyl, C₁-C₆ haloalkyl, —NO₂, —CN, —OH, —SH, —NH₂, —NH(C₁-C₆ alkyl), —N(C₁-C₆ alkyl)₂, C₁-C₆ alkoxy, or C₁-C₆ haloalkoxy;
R⁴ is hydrogen, halogen, C₁-C₆ alkyl, or C₁-C₆ haloalkyl; and
R is C₁-C₆ alkyl, aryl optionally substituted with one or more R₅, or heteroaryl optionally substituted with one or more R₅,
wherein
each R₅ is independently selected from the group consisting of halogen, —NO₂, —CN, C₁-C₆ alkyl, C₁-C₆ haloalkyl, —OH, C₁-C₆ alkoxy, and C₁-C₆ haloalkoxy.

5. The method of claim 4, wherein R¹ is hydrogen or C₁-C₃ alkyl.

6. The method of claim 4, wherein R¹ is hydrogen or methyl.

7. The method of claim 4, wherein R¹ is hydrogen.

8. The method of any of claims 4-7, wherein R² is hydrogen, halogen, C₁-C₃ alkyl, C₁-C₃ haloalkyl, —NO₂, —CN, —OH, —SH, —NH₂, —NH(C₁-C₃ alkyl), —N(C₁-C₃ alkyl)₂. C₁-C₃ alkoxy, C₁-C₃ haloalkoxy, or phenyl optionally substituted with one or more R₅.

9. The method of any of claims 4-7, wherein R² is hydrogen, halogen, C₁-C₃ alkyl, C₁-C₃ haloalkyl, —NO₂, —OH, —NH₂, —NH(C₁-C₃ alkyl), —N(C₁-C₃ alkyl)₂, C₁-C₃ alkoxy, or C₁-C₃ haloalkoxy.

10. The method of any of claims 4-7, wherein R² is hydrogen, halogen, C₁-C₃ alkyl, C₁-C₃ haloalkyl, —NO₂, C₁-C₃ haloalkoxy or phenyl.

11. The method of any of claims 4-7, wherein R² is hydrogen, halogen, C₁-C₃ alkyl, or C₁-C₃ haloalkyl.

12. The method of any of claims 4-7, wherein R² is halogen, C₁-C₃ alkyl, or C₁-C₃ haloalkyl.

13. The method of any of claims 4-7, wherein R² is halogen or C₁-C₃ haloalkyl.

14. The method of any of claims 4-7, wherein R² is bromo, chloro. or —CF₃.

15. The method of any of claims 4-7, wherein R² is —CF₃.

16. The method of any of claims 4-15, wherein R³ is hydrogen, halogen, C₁-C₃ alkyl, or C₁-C₃ haloalkyl.

17. The method of any of claims 4-15, wherein R³ is hydrogen, bromo, chloro, methyl, or —CF₃.

18. The method of any of claims 4-15, wherein R³ is hydrogen.

19. The method of any of claims 4-18, wherein R⁴ is hydrogen or C₁-C₃ alkyl.

20. The method of any of claims 4-18, wherein R⁴ is hydrogen.

21. The method of claim 4, wherein R¹ is hydrogen, R² is halogen, C₁-C₃ alkyl, or C₁-C₃ haloalkyl, R³ is hydrogen, and R⁴ is hydrogen.

22. The method of any of claims 4-21, wherein R is phenyl or 6-membered heteroaryl, each optionally substituted with one or more R₅.

23. The method of any of claims 4-21, wherein R is phenyl, pyridinyl, or pyrimidinyl, each optionally substituted with one or more R₅.

24. The method of any of claims 4-21, wherein R is phenyl, pyridinyl, or pyrimidinyl, each optionally substituted with one or two R₅.

25. The method of any of claims 4-21, wherein R is

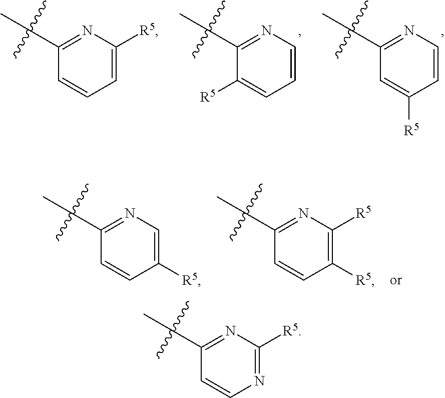

26. The method of any of claims 4-25, wherein R⁵ is halogen, C₁-C₆ alkyl, C₁-C₆ haloalkyl, C₁-C₆ alkoxy, and C₁-C₆ haloalkoxy.

27. The method of any of claims 4-25, wherein R⁵ is halogen, C₁-C₃ alkyl, C₁-C₃ haloalkyl, C₁-C₃ alkoxy, and C₁-C₃ haloalkoxy.

28. The method of any of claims 4-25, wherein R⁵ is bromo, chloro, methyl, —CF₃, or methoxy.

29. The method of any of claims 4-25, wherein R⁵ is chloro, methyl, or methoxy.

30. The method of any of claims 4-29, wherein R is

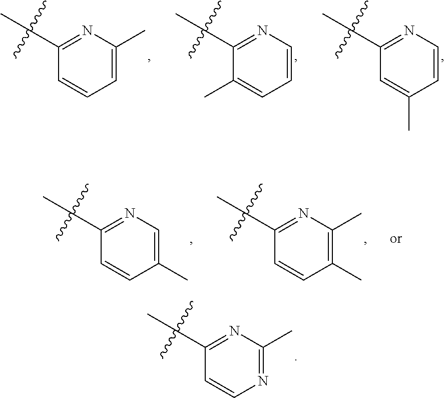

31. The method of any of claims 4 to 21, wherein R is

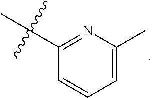 [SMILES: *c1cccc(C)n1]

32. The method of any one of claims 4-31, wherein the compound is: 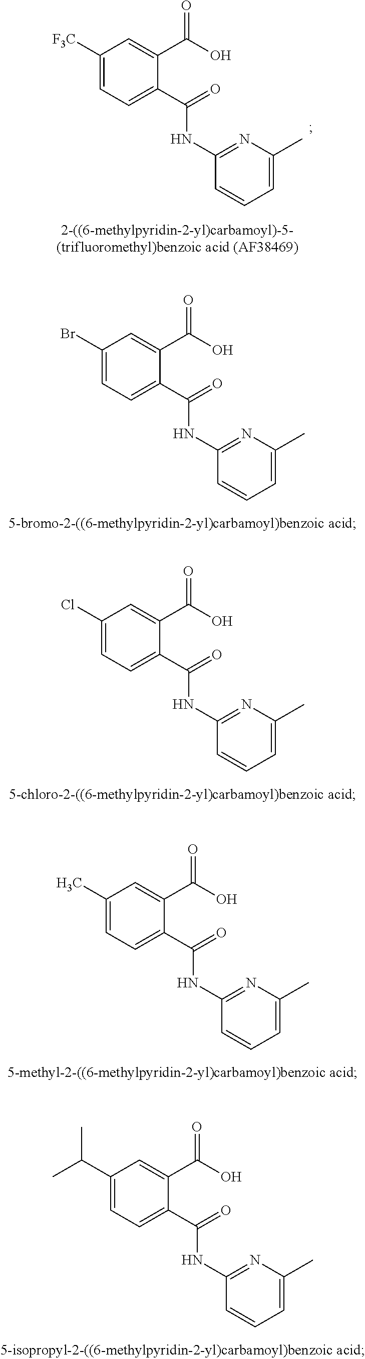 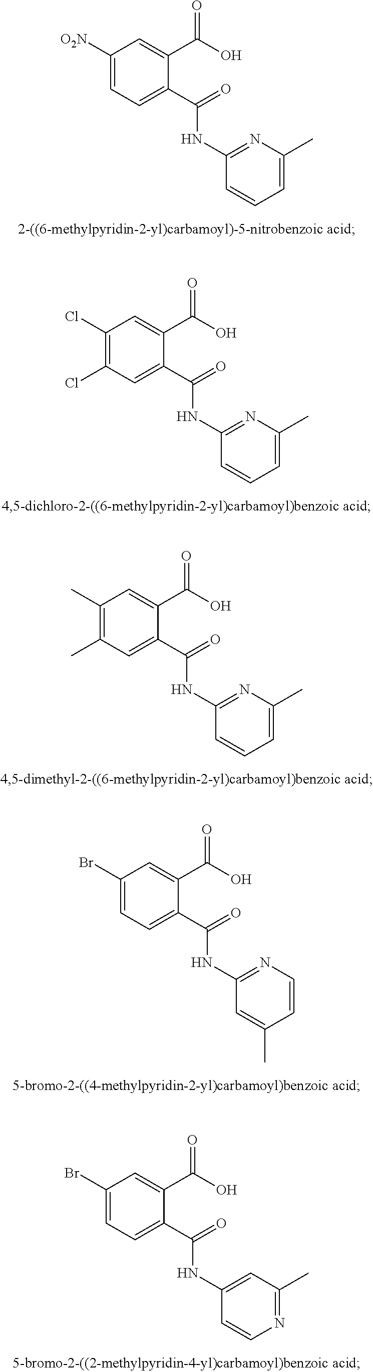 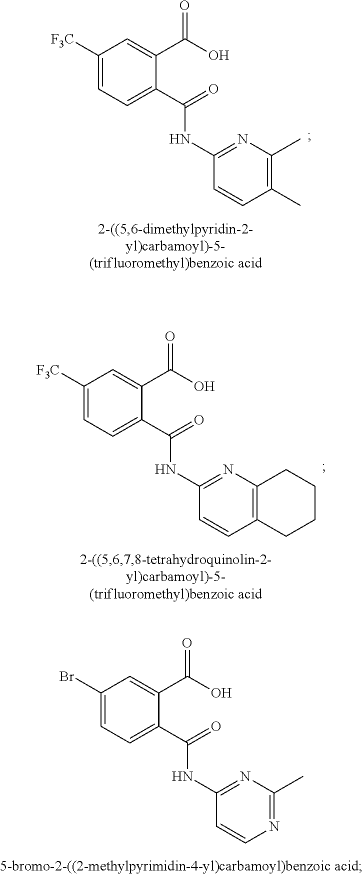 [SMILES: Cc1ccc(NC(=O)c2ccc(C(F)(F)F)cc2C(=O)O)nc1C ; O=C(O)c1cc(C(F)(F)F)ccc1C(=O)Nc1ccc2c(n1)CCCC2 ; Cc1nccc(NC(=O)c2ccc(Br)cc2C(=O)O)n1]
or pharmaceutically acceptable salts thereof.

33. The method of any one of claims 1-32, wherein the SORT1 inhibitor is: 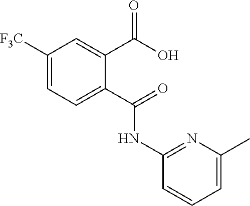 [SMILES: Cc1cccc(NC(=O)c2ccc(C(F)(F)F)cc2C(=O)O)n1]
2-((6-methylpyridin-2-yl)carbamoyl)-5-(trifluoromethyl)benzoic acid (AF38469), or pharmaceutically acceptable salts thereof.

34. The method of any one of claims 1-3, wherein the SORT1 inhibitor comprises an inhibitor selected from the group consisting of AF38469 or N-substituted-5-substituted pthalamic acids, AF40431 (N-[(7-hydroxy-4-methyl-2-oxo-2H-chromen-8-yl)methyl]-L-leucine) or substituted versions thereof; (S)-2-(3,5-dichlorobenzamido)-5,5-dimethylhexanoic acid or derivatives such as (S)-2-(4-chloro-1H-pyrrole-2-carboxamido)-5,5-dimethylhexanoic acid and (S)-5-5-dimethyl-2-(6-phenoxynicotinamido)hexanoic acid or substituted versions thereof; 1-benzyl-3-(tert-butyl)-1H-pyrazole-5-carboxylic acid or substituted versions thereof, SORT1 small interfering RNAs, small internally segmented interfering RNAs, short hairpin RNAs, microRNAs, and/or antisense oligonucleotides; cas9 repressors or other cas (CRISPR) repressors targeting the SORT1 locus; anti-SORT1 antibodies or antibody fragments thereof; combinations thereof; or pharmaceutically acceptable salts thereof; in particular AF38469 or a pharmaceutically acceptable salt thereof.

35. The method of any one of claims 1 to 34, wherein the compound is a salt.

36. The method of any one of claims 1 to 35, wherein the compound is administered as a pharmaceutical composition comprising the compound and a pharmaceutically acceptable carrier, solvent, adjuvant or diluent.

37. The method of any one of claims 1-36, wherein the lysosomal storage disorder is selected from the group consisting of NCL/Batten Disease caused by mutations in CLN gene CLN1 (PPT1), CLN2 (TPP1), CLN3, CLN4 (DNAJC5), CLN5, CLN6, CLN7 (MFSD8), CLN8, CLN10 (CTSD), CLN11, CLN12 (ATP13A2), CLN13 (CTSF), CLN14 (KCTD7)), CLCN6, and/or SGSH; Pompe disease, Fabry disease, Gaucher disease, Niemann-Pick disease Types A, B, and C; GM1 gangliosidosis, GM2 gangliosidosis (including Sandhoff and Tay-Sachs), mucopolysachariddoses (MPS) types I (Hurler disease)/II (Hunter disease)/IIIa (Sanfilippo A)/IIIB (Sanfilippo B)/IIIc (Sanfilippo C)/IIId (Sanfilippo D)/IVA (Morquio A)/IVB/VI/VII (Sly)/IX, mucolipisosis III (I-cell) and IV, multiple sulfatase deficiency; sialidosis, galactosialidosis, α-mannosidosis, β-mannosidosis, apartylglucosaminuria, fucosidosis, Schindler disease, metachromatic leukodystrophy caused by deficiencies in either arylsulfatase A or Saposin B, globoid cell leukodystrophy (Krabbe disease), Farber lipogranulomatosis, Wolman and cholesteryl ester storage disease, pycnodystostosis, cystinosis, Salla disease, Danon disease, Griscelli disease Types 1/2/3, Hermansky Pudliak Disease, and Chédiak-Higashi syndrome.

38. The method of any one of claims 1-37, wherein the lysosomal storage disorder comprises NCL/Batten Disease caused by mutations in one or more genes selected from the group consisting of CLN1 (PPT1), CLN2 (TPP1), CLN3, CLN4 (DNAJC5), CLN5, CLN6, CLN7 (MFSD8), CLN8, CLN10 (CTSD), CLN11, CLN12 (ATP13A2), CLN13 (CTSF), CLN14 (KCTD7), CLCN6, and/or SGSH.

39. The method of any one of claims claim 1-38, wherein the method further comprises administering to the subject an amount effective of a p75 neurotrophin receptor (NGFR) modulator, and/or a glucagon-like peptide-1 receptor (GLP-1R) agonist.

40. The method of any one of claims 1-39, wherein the method comprises administering to the subject an amount effective of AF38469 (2-((6-methylpyridin-2-yl)carbamoyl)-5-(trifluoromethyl)benzoic acid) or a pharmaceutically acceptable salt thereof, and LM11A-31 (N-[2-(morpholin-4-yl)ethyl]-L-isoleucinamide) or a pharmaceutically acceptable salt thereof, to treat the and/or the lysosomal storage disorder.

41. The method of any one of claims 1-40, wherein the method further comprises administering to the subject an amount effective of semaglutide, or a pharmaceutically acceptable salt thereof, to treat the lysosomal storage disorder.

42. The method of any one of claims 1-40, wherein the method comprises administering to the subject an amount effective of AF38469 (2-((6-methylpyridin-2-yl)carbamoyl)-5-(trifluoromethyl)benzoic acid) or a pharmaceutically acceptable salt thereof, and an amount effective of semaglutide, or a pharmaceutically acceptable salt thereof, to treat the lysosomal storage disorder.

43. The method of any one of claims 1-42, wherein the method further comprises administering one or more gene therapy products that encode proteins implicated in lysosomal storage disorders, including but not limited to gene therapy product encoding PPT1, TPP1, CLN3, CLN4 (DNAJC5), CLN5, CLN6, CLN7 (MFSD8), CLN8, CLN10 (CTSD), CLN11, CLN12 (ATP13A2), CLN13 (CTSF), CLN14 (KCTD7) lysosomal alpha-glucosidase (GAA), alpha-galactosidase A (GLA), glucosylceramidase beta (GBA), acid sphingomyelinase (SMPD1), NPC intracellular cholesterol transporter 1 (NPC1), NPC intracellular cholesterol transporter 2 (NPC2), beta-galactosidase 1 (GLB1), beta-hexosamidase A (HEXA), GM2 ganglioside activator (GM2A), alpha-L iduronidase (IDUA), iduronate 2-sulfatase (IDS), N-sulfoglucosamine sulfohydrolase (SGSH), N-acetylglucosaminidase (NAGLU), heparan-alpha-glucosaminide N-acetyltransferase (HGSNAT), N-acetylglucosamine-6-sulfatase (GNS), N-acetylgalactosamine-6-sulfatase (GALNS), arylsulfatase B (ARSB), beta-glucoronidase (GUSB), hyaluronidase 1 (HYAL1), N-acetylglucosamine-1-phosphate transferase subunits alpha and beta (GNPTAB), mucolipin 1 (MCOLN1), sulfatase modifying factor 1 (SUMF1), neuraminidase 1 (NEU), cathepsin A (CTSA), alpha-mannosidase (MAN2B1), beta-mannosidase (MANBA), aspartylglucosaminidase (AGA), alpha-L-fusocidase (FUCA1), alpha-N-acetylgalactosaminidase (NAGA), arylsulfatase A (ARSA), prosaposin (PSAP), galactosylceramidase (GALC), acid ceramidase 1 (ASAH1), lipase A (LAL), cathepsin K (CTSK), cystinosin (CTNS), solute carrier family 17 member 5 (SLC17A5), lysosomal associated membrane protein-2 (LAMP2), myosin VA (MYO5A), Rab27a member RAS oncogene family (RAB27A), melanophilin (MLPH), biogenesis of lysosomal organelles complex 3 subunit 1 (HPS1), AP-2 complex subunit beta-1 (AP3B1), biogenesis of lysosomal organelles complex 2 subunit 1 (HPS3), biogenesis of lysosomal organelles complex 3 subunit 2 (HPS4), biogenesis of lysosomal organelles complex 2 subunit 2 (HPS5), biogenesis of lysosomal organelles complex 2 subunit 3 (HPS6), dystrobrevin binding protein 1 (DTNBP1), biogenesis of lysosomal organelles complex 1 subunit 3 (BLOCIS3, PLDN) adaptor related protein complex 3 subunit delta 1 (AP3D1), lysosomal trafficking regulator (LYST), or functional fragments thereof.

44. A pharmaceutical composition, comprising:
(a) a SORT1 inhibitor as recited in any one of claims 4-34; and
(b) 1, 2, or all 3 of
(i) a NGFR modulator,
(ii) a GLP-1R agonist; and
(iii) a nucleic acid encoding a gene therapy expression product capable of substituting for a protein deficient in a lysosomal storage disorder and/or neurological disorder; and
(c) a pharmaceutically acceptable carrier.

45. The pharmaceutical composition of claim 44, wherein the composition comprises the gene therapy product, and wherein the gene therapy product is capable of substituting for a protein deficient in a lysosomal storage disorder and/or neurological disorder.

46. The pharmaceutical composition of claim 45, wherein the gene therapy product encodes PPT1, TPP1, CLN3, CLN4 (DNAJC5), CLN5, CLN6, CLN7 (MFSD8), CLN8, CLN10 (CTSD), CLN11, CLN12 (ATP13A2), CLN13 (CTSF), CLN14 (KCTD7) or functional fragment thereof.

47. The pharmaceutical composition of claim 45 or 46, wherein the gene therapy product encodes one or more additional gene therapy products that encode proteins implicated in lysosomal storage disorders, including but not limited to lysosomal alpha-glucosidase (GAA), alpha-galactosidase A (GLA), glucosylceramidase beta (GBA), acid sphingomyelinase (SMPD1), NPC intracellular cholesterol transporter 1 (NPC1), NPC intracellular cholesterol transporter 2 (NPC2), beta-galactosidase 1 (GLB1), beta-hexosamidase A (HEXA), GM2 ganglioside activator (GM2A), alpha-L iduronidase (IDUA), iduronate 2-sulfatase (IDS), N-sulfoglucosamine sulfohydrolase (SGSH), N-acetylglucosaminidase (NAGLU), heparan-alpha-glucosaminide N-acetyltransferase (HGSNAT), N-acetylglucosamine-6-sulfatase (GNS), N-acetylgalactosamine-6-sulfatase (GALNS), arylsulfatase B (ARSB), beta-glucoronidase (GUSB), hyaluronidase 1 (HYAL1), N-acetylglucosamine-1-phosphate transferase subunits alpha and beta (GNPTAB), mucolipin 1 (MCOLN1), sulfatase modifying factor 1 (SUMF1), neuraminidase 1 (NEU), cathepsin A (CTSA), alpha-mannosidase (MAN2B1), beta-mannosidase (MANBA), aspartylglucosaminidase (AGA), alpha-L-fusocidase (FUCA1), alpha-N-acetylgalactosaminidase (NAGA), arylsulfatase A (ARSA), prosaposin (PSAP), galactosylceramidase (GALC), acid ceramidase 1 (ASAH1), lipase A (LAL), cathepsin K (CTSK), cystinosin (CTNS), solute carrier family 17 member 5 (SLC17A5), lysosomal associated membrane protein-2 (LAMP2), myosin VA (MYO5A), Rab27a member RAS oncogene family (RAB27A), melanophilin (MLPH), biogenesis of lysosomal organelles complex 3 subunit 1 (HPS1), AP-2 complex subunit beta-1 (AP3B1), biogenesis of lysosomal organelles complex 2 subunit 1 (HPS3), biogenesis of lysosomal organelles complex 3 subunit 2 (HPS4), biogenesis of lysosomal organelles complex 2 subunit 2 (HPS5), biogenesis of lysosomal organelles complex 2 subunit 3 (HPS6), dystrobrevin binding protein 1 (DTNBP1), biogenesis of lysosomal organelles complex 1 subunit 3 (BLOCIS3, PLDN) adaptor related protein complex 3 subunit delta 1 (AP3D1), lysosomal trafficking regulator (LYST), or functional fragments thereof.

48. The pharmaceutical composition of any one of claims 44-47, wherein the composition comprises a NGFR modulator.

49. The pharmaceutical composition of any one of claims 44-48, wherein the composition comprises a GLP-1R agonist.

50. A pharmaceutical composition, comprising:
(a) a SORT1 inhibitor of any one of claims 4-34;
(b) one or both of:
(i) a NGFR modulator, and
(ii) a GLP-1R agonist; and
(c) a pharmaceutically acceptable carrier.

51. The composition of any one of claims 44-50, wherein the composition comprises AF38469 (2-((6-methylpyridin-2-yl)carbamoyl)-5-(trifluoromethyl)benzoic acid) or a pharmaceutically acceptable salt thereof.

52. The composition of claim 51, wherein the composition further comprises LM11A-31 (N-[2-(morpholin-4-yl)ethyl]-L-isoleucinamide) or a pharmaceutically acceptable salt thereof.

53. The composition of any one of claims 51-52, wherein the composition further comprises semaglutide or a pharmaceutically acceptable salt thereof.


SEQUENCE LISTING (3 sequences)

SEQ ID NO: 1 - protein, 37 aa, Artificial Sequence
  Synthetic
HDEFERHAEGTFTSDVSSYLEGQAAKEFIAWLVKGRG

SEQ ID NO: 2 - protein, 39 aa, Artificial Sequence
  Synthetic
HGEGTFTSDLSKQMEEEAVRLFIEWLKNGGPSSGAPPPS

SEQ ID NO: 3 - protein, 30 aa, Artificial Sequence
  Synthetic
HAEGTFTSDVSSYLEGNAAKEFIAWLVKGR